SEQUENCE LISTING (85 sequences)

SEQ ID NO: 1 - DNA, 30 bp, Artificial Sequence
  Description of Artificial Sequence Hypothetical allele
gggaatatttattacctatsttatattgga

SEQ ID NO: 2 - DNA, 30 bp, Artificial Sequence
  Description of Artificial Sequence Hypothetical allele
gggaatatttattacctatgttatattgga

SEQ ID NO: 3 - DNA, 30 bp, Artificial Sequence
  Description of Artificial Sequence Hypothetical allele
gggaatatttattacctatcttatattgga

SEQ ID NO: 4 - DNA, 30 bp, Artificial Sequence
  Description of Artificial Sequence Hypothetical allele
gggaatatttattacctatcttataytgga

SEQ ID NO: 5 - DNA, 30 bp, Artificial Sequence; identical to SEQ ID NO: 2
  Description of Artificial Sequence Hypothetical allele

SEQ ID NO: 6 - DNA, 30 bp, Artificial Sequence; identical to SEQ ID NO: 3
  Description of Artificial Sequence Hypothetical allele

SEQ ID NO: 7 - DNA, 30 bp, Artificial Sequence
  Description of Artificial Sequence Hypothetical allele
gggaatatttattacctatcttatactgga

SEQ ID NO: 8 - DNA, 23 bp, Sus scrofa
ctgacagttaaagactgcccaac

SEQ ID NO: 9 - DNA, 24 bp, Sus scrofa
agctccatgtcttactcatccacc

SEQ ID NO: 10 - DNA, 24 bp, Sus scrofa
gcagcaatactgtgccttagaaac

SEQ ID NO: 11 - DNA, 24 bp, Sus scrofa
aagtaatagggagtgagagtctcc

SEQ ID NO: 12 - DNA, 23 bp, Sus scrofa
atctcgacaacctcaagggcacc

SEQ ID NO: 13 - DNA, 24 bp, Sus scrofa
ccactcacatgcgtgctttacaac

SEQ ID NO: 14 - DNA, 23 bp, Sus scrofa
tggtgcctggtctgatgatgtac

SEQ ID NO: 15 - DNA, 24 bp, Sus scrofa
agcagaaagcgcttgcggtattca

SEQ ID NO: 16 - DNA, 24 bp, Sus scrofa
caccctgcagcatcttcttagctg

SEQ ID NO: 17 - DNA, 24 bp, Sus scrofa
ccaaactgaggccgggttgtgctc

SEQ ID NO: 18 - DNA, 22 bp, Sus scrofa
tgcaagcatccagggctgcttt

SEQ ID NO: 19 - DNA, 24 bp, Sus scrofa
cgaaacaagttctagtgagctctg

SEQ ID NO: 20 - DNA, 23 bp, Sus scrofa
acacggagtcagagttcacagag

SEQ ID NO: 21 - DNA, 24 bp, Sus scrofa
agcagaagtaaggtcctggctgaa

SEQ ID NO: 22 - DNA, 22 bp, Sus scrofa
aaaccgctgcctccatccagtg

SEQ ID NO: 23 - DNA, 24 bp, Sus scrofa
aggctggagtactccaattactcc

SEQ ID NO: 24 - DNA, 24 bp, Sus scrofa
tccatgataggctgcatcctagaa

SEQ ID NO: 25 - DNA, 24 bp, Sus scrofa
ccagaggtcacgggaacagaactg

SEQ ID NO: 26 - DNA, 24 bp, Sus scrofa
tccctgttctctatggcctgcttc

SEQ ID NO: 27 - DNA, 24 bp, Sus scrofa
gatgtggtggtgctgaactccaag

SEQ ID NO: 28 - DNA, 24 bp, Sus scrofa
tctgagcctcatattctaatggac

SEQ ID NO: 29 - DNA, 23 bp, Sus scrofa
cctttcactgcagaagttccagg

SEQ ID NO: 30 - DNA, 25 bp, Sus scrofa
gattgcagtattttttgtcttggag

SEQ ID NO: 31 - DNA, 23 bp, Sus scrofa
ctgaaagatccatccatttgttc

SEQ ID NO: 32 - DNA, 24 bp, Sus scrofa
atgctgcgatttctctggagttcc

SEQ ID NO: 33 - DNA, 24 bp, Sus scrofa
tgtccagacgttccctttggctcc

SEQ ID NO: 34 - DNA, 26 bp, Sus scrofa
ttgaaatccctacttaagtcctgctc

SEQ ID NO: 35 - DNA, 27 bp, Sus scrofa
caagagtaaatcatgcaaggaaatgtg

SEQ ID NO: 36 - DNA, 25 bp, Sus scrofa
taggacaggagaaggataacaaacc

SEQ ID NO: 37 - DNA, 24 bp, Sus scrofa
agtccctggacatatgcaggttag

SEQ ID NO: 38 - DNA, 25 bp, Sus scrofa
agatgatcatcgagctgtaggatag

SEQ ID NO: 39 - DNA, 25 bp, Sus scrofa
ttccaatcctttgtgaatatctggc

SEQ ID NO: 40 - DNA, 24 bp, Sus scrofa
gcagggacagctctgccagggaac

SEQ ID NO: 41 - DNA, 27 bp, Sus scrofa
gttaccttacttgaaccctttctttcc

SEQ ID NO: 42 - DNA, 25 bp, Sus scrofa
agagaaccctcaactctcagctgtg

SEQ ID NO: 43 - DNA, 26 bp, Sus scrofa
ttcagccttattgaggtatagttatc

SEQ ID NO: 44 - DNA, 23 bp, Sus scrofa
caagaagcagagcttcgtgggtg

SEQ ID NO: 45 - DNA, 24 bp, Sus scrofa
cgatgagtccatgagcttagaacc

SEQ ID NO: 46 - DNA, 25 bp, Sus scrofa
gtgtaatctgttggagtatttctgt

SEQ ID NO: 47 - DNA, 24 bp, Sus scrofa
tcggtaagattatcatctgacttc

SEQ ID NO: 48 - DNA, 23 bp, Sus scrofa
tgagcagcaggtctggaatctag

SEQ ID NO: 49 - DNA, 24 bp, Sus scrofa
gagtagccacaagaatataccaag

SEQ ID NO: 50 - DNA, 23 bp, Sus scrofa
ccacaatgcttgcctttgcagag

SEQ ID NO: 51 - DNA, 24 bp, Sus scrofa
cgcatcacacaaatgtgttcatgg

SEQ ID NO: 52 - DNA, 23 bp, Sus scrofa
gagaggcagtgggtccagaccaa

SEQ ID NO: 53 - DNA, 24 bp, Sus scrofa
tccaaggtggtctgagctgacctg

SEQ ID NO: 54 - DNA, 659 bp, Sus scrofa
ctgacagttaaagactgcccaacagtgaagtgaactgcctaaaaaacagtgagttttctattttttatgtgttcaaatgaaggaaaaataaatctgtccattatggggataaggrgataccagtgttcaaggggagttaaaacaaaaagatttctaatgtaccttcaaattcttaagattccatgaaactgtgaatttatataggaataaaaaagtgaaactattctcttgatatgcaaagatgaggaaaaaagatttacatgataaaayttcaaaataaatcgtttgccattttaagctgtattgttcgagctcaagaaccttctttaacaatatttccatctttctaatttataattatccaataaaatatacaattacctaccactccaaattttaaagtaaatatgtttgagatcaatgtgcagatgaaaggtyttatttgtataagaggaaagatagtgctatgtaaatacccctttcccatcaagtatattcctatgcacttccataaaggcaattcagtgtgtattttcacaggatccttggttattggttcattttaggtctactgacgaagcagaccttcagaaaaatatttaccctgaattagagsatcaagatggtggatgagtaagacatggagcttaccttcttcc

SEQ ID NO: 55 - DNA, 795 bp, Sus scrofa
  misc_feature (1)..(795): nucleotides 121-915 of ACY-STS7
cagcaggatttttgctgagtttttttggaaaccccctcaggaccaacccccacccccccaaaaagtattaagcaccaaagttaatagaagagattcacagcaaacaaggcagaaccagagaccasgggtacaggggagacaacaaaccaggaccagggctcaatctttctgctcccaccctacagcctcagtcttctatgctaatcctgagaaatccctagcatgggaagggacactgcaaagcactgtactgacctagcactggatcagatcaaggtcatatggctggtcaatgagcaaygtaaaacttacaaggtactgggtacacacagccaagggcatcccttcccttgaaaagctcttaagccagggagataagacaacctgccctcagaaggcaggttacacttgcctagggggttataccctggcccagtaaaggtcaggcaaagctttactatgggcctggcagagcatgaagtccaggcaaaagctggctaggcagagaaaaattgtgggctttggtaggccaagtaagatgaaggacacttaataataatagcactcaggcaggggctgaagccagcaaaggctacatgaaagatcctgactgtgcaaatggagccaaagagacacacttctgtgtgattccgagcacaaactcacccccttaagagattcatatcgttgtgatcggggtttcttgatgccgtttctgtgccattttcgggctgtggagaagtaaagcattaggtcaacaaaatagatttcccccaataagaccactaccccagc

SEQ ID NO: 56 - DNA, 1034 bp, Sus scrofa
ggtccctgygggtycacgtgggttggtgtctacccgtcttcacaagctggtactgatttggtacgttctctgccttatgggttctgtgctactgatctatatgtcttcctggttcattcatgcctgaggtgctttagattagttggcattgtttacgggtaataccaacagttaacacttatacccagaactcaccacgtcccggggcacagctgcactgcgtgtatataaattccttcgcccctaaggggaggtacttctatgaaccctgctttactaacgaccaaatggagcccagacgtcaggtcgctttacrgcacatagtgacttgatcccagggtggctctgctgccacttgccgatctgtcttggttgacatgggctgggctgtcccttagagtcagacctttccccagggcaaaggccactacaagtcaggggcctaagcagcaaagctgaccatggcctcgccagctcaccagccttccctggctccctgttgcctgcagggtgtggtcctgctcrggcgcttcctgttttcctctccaagacttcttccctcactctgcccaaaacatccttcttccccttctgcatcccaccagctccaacgtaggcttcaagatgyctcctccaggaagtcctcccagctgtgctcctctccacactccccgctcagttgatgtctccrccgcacrcacgtccctcatccagcacttcctgtgacagtgcttctccccctgcatctccccccgtgagcctcagactggccrttcccggaagagcrgtgayrtggatgagtgrcccagagttagcracctagagctgaggggccatctcccagtcctgtggcccttactccccagccgcaccccctyggrcagggagcacagggagggctgctggtgtgttctagccatggcccgatgaccyttgcygcctccccatgctgtgttcctgggctggggaagggtctccacagggaagggagaggttgacaggagagccccctgcccctaytgccctggggacac

SEQ ID NO: 57 - DNA, 833 bp, Sus scrofa
gaccaagagctgcagcaccgtgtggagtccctggcagcctttgcagaacgctacgtggacaagctccaggccaaccagagggaccgctatggcatcctcatgaaggccttcaccatgaccgctgccgagactgcccgacgtactcgcgagttccgctccccaccccaggagcaggtcctctaacccccaaactcagctggccttactgtctcaacctcagcctctccccttactctgatcactgatggcactcaacctctaaacctgggcttgacctctgatcctgtgggtatacttctctcttgctcccctacctctctctgacccagatttcrgagtcagcccagactgaccctaagtcctttcaaacctttgatctcccagatattcctcagtaactcmtgactscagacaggggctcagttggatcttagatccttgacctcagagttcctgctccggggtctctgaccctcattctaacctttgaccttccctagatcaacatgctattgcacttcaaagatggcgaggatgaggaagattgtcctcttcctgatgagatcmggcaggatttgctggaattccatcaagacctgttgactcactgtggtaagagaggatatcagggaatcctcttccccagttttttctcgagacctctctgaaagtttccctaagatttcctgatcttggagttcccgtcgtggcgcagtggttmacgaatccgactaggaaccatgaggttgcgggttcggtccctgcccttgctcagtgggttamygatccggcgttgccgtgagctgtggtgtaaggttgcaaacccagctcaa

SEQ ID NO: 58 - DNA, 639 bp, Sus scrofa
ttctaaagttcagcatacttcactagtgatacatgtcttaytgatacttccttaagagttatgtgcttacctgcctaggccctccctccactagatggctcagccctggggatcaggygttatctccttagctgcatgaagctrgagtmgtgtgttgtgcacaccagaatccacctgcgttcaacacctagctctggagctcctgctatggaccaggcattgtttctggtgccrctgatgcagyggagagcaaatcagatcccacaaacccatgaygctcrcatgtgcatracggaggaaaaaatatcaaggaagaagcaaatgaaatgagaatacaggactcctgacctagtccctactarccagaagttgtctccaaagrttttcattttctatgccygtggatgatggtcagaaagaagatctgcctgtaatcatgttctcgaagggatccaagacctymagcaaccagaaaggaactacttcacagtaayactgttccaaaccaacagtaagatgcccgttccctcacttcgcttccatcttctttaacrtcaagcagtccttggagctagctacttcttagtcgtaagaactcgaacgtacataacgtatttgcagtttccaaagcacatttcc

SEQ ID NO: 59 - DNA, 672 bp, Sus scrofa
cctgaagaattgatacattaatgtgctatttcatagcgtgataagaatgtgcctttgccatctcctttgaaatgcaaacatctttattctttaggggagactttgtttactttgattcaacagtgmaaaaaattgggaattagaaacctttctgtagtttcccagaagctggctcttagcacagatttttggtttctctcactggagttgacttgcatcgaaagttgggagcaaccctaaaaggtatgacctaaaatcaagctggtggcagagtaggggagtctgtacatagcgccgggggttctgagcgtgctgtagtgtgtgtastgtakttctsmgtgtggggaatcctcaagacagggagtccyaggggcctgtaggtattcctcttcctgaaatcatggaatgggtgagccggaaggagaaccttctattctttgctggccttatttctttttckttccctctcamaagttacagaggtggcttcacagatccaggtcctgctggagatcttcggctgyccctgagaakccaggaagatttttactaaaaayttactyttccatctcctctgctasaaaggccgccactgtcgctttggcctccacagaggccacaacaccctcagctccagagtcttcactgaatgtacctgctttcacatgaaca

SEQ ID NO: 60 - DNA, 1845 bp, Sus scrofa
ggatccttaacccactgagtgaggccagggatcgaatttgcattctcgtagatactggtcagatttgtttctgctgggccaccatgggaactccctggttttgtctatatatatttttttttttttttgtcttttttgccatttcttgggccgctcctgcggcatatggaggttcccaggctaggggtcgaatcggagctgtagccaccagcctacgccagagccacagcaacgtgggacccgagccgagtctgcaacctataccacagctcacggcaacgccagatcccttaacccactgagcaaggccagggaccgaacccgcaacctcatggttcttagtcggattcgttaaccactgcgccacgacgggaactcccggttttgtctatttttgaacgttaaataaatgcaagcatccagggctgctttgactcagtaccatgtgtgagatttaccctgttgatgtcagcagctrtggctggttccttctcacggatgtgtgtgaccctcacctggaccacacctgatctggctgatgatgggccttggggtttttccagcttttggtcccakgtcacgtctctgtttgaacttaaatgcacttgctttcaggtattaatctggggcggaatgactggaacatgaggtgtggttggttcagctttagtagatgccagcagggaggatttcagtagtttattaagcagatcttgaagactgtggtcaactagctcatgccccagaggagggggcgstgaatttcttccccagaacaggagtgagaagctaaattaggcatccatccgctggaagytgagggggcagttcttggctcctttctgtcaggtttcggccccttctcsttagtctggggtttctaggctctactcccaggaagwgtctggggccacttgggaagaatgggtgggggggctytgagcccctacttacttcatttccctccttcagccaaarccycctgtgtcctctgttttacatagtggggttctgagaatgacttywtttttttttttttttttttwaaagctttagctrtkgcgacatttacaaatccmctgctgtgaggtctcttccagctaggaaattctattttggrascagraggtgggtgtgggragggttaagcattattcagccaaagagttgggttgggcctcagtgaccttttgaagttcttatagcttggcttgccatgcaggagatctcagaacattctataaaaatagtgttcaaacagaacaacttctgaagcctaaaggatgcgaacaagaggctcggaaggtagcatttcaacgggagttttgaggatgctctcctttagccacccctctccattttctgcccccttctttttaaattctccattggctgtccctgctagttgtcatttggggtggtttgggttcagaatggttctcattttcgccgaggagtgggtgatgtgggcggcctgtgtgtctctcccaagggtggtggctgtccctcctccaccaccaggcctagtttggacctgtagtttcgcttagtgaaggaggccgggccgatcctgggccggagagagacgtmtcatgccwtggcatgcagctctgagtcaacaggcctgataaacagcccacttcccagggcgagcaaggaggaacaaggcccctggctgctgtgggatccgtctgcgctcctcttcgtgaaaccgctgtttattcttttgacaggagttggaacgcagcaccttcccttcctcccagccctgcctccttctgcagagcagagctcactagaacttgtttcgccttttactctggggggagagaagcagaggatgag

SEQ ID NO: 61 - DNA, 1260 bp, Sus scrofa
gaatttgtctcagtarttaatgactttaaagctrcaaaagaattaagaaagaaatagctattaccaggccaggaagataaaaaccttatcagagacaatatatcagttgtgaagaatcctggttctgttttaaagataaaagttagmcttamggcacattgcttaaatkgttttacagctcaaccagcccatcaagtactcaaccaccaggccaagtggaacctaagaaaggatgatgccagccttggctgacccttgtaactctaatcagctggacctttgccccagttctatgctgaattcttcyttgctcaagccctttcatgagtatgaatgtacccttaryttaaaacttccccagttttgctgtttgagagacattgttttgggaactatccctgatactcttcttactgttaagtaaaacaaatccttcctactcctgctctttggcttgactgtgtcttttggctcaaaacccaaygagaggtgaacccagttttggggtgacattagggatcaggtggatcaactcaggcatmgtaggaaaagctactggtgttccaccagttccagcttaagctcatggatggcattctcyagcaagaatygcaattgtccacctaaagatgttctctcagcttctgtgggccaagtaggccagaaatgccagattrggagttcctgtggtagctcagcagattaagaaccctactcagtktctgygaggatgcaggtttgttcctgaagattargttcttgtctagttgcagaaggaattcagaaatgagatggaaattgagaaagaaaagtgagggtttatttaagtgagaagtacacctcttaaggagaggtgggcagagaggtgggcagagaggtgagcagctgycctgtgttttttgggtgcactagttagaaggggtgtccaattgtatagatgggatagtcaytgagaaagtggggtttaggggtcatattccttaattttcaycccagctccaccttcccraagggaggagggatttttgtccttagttggttaattggaagtgtcatggcatccayrtataatgggtacttcttatctgcatagctaattgtattakaattttattataaggagggcataatgagcaacagkgttccattcagacactggagattcgkgccctcttctacctttctttgtctgcagcctgggcacttatcaccccaaaaatgtgtgrtttcctatcagtctggtgkttccrgcttttcttt

SEQ ID NO: 62 - DNA, 1444 bp, Sus scrofa
  misc_feature (1)..(1444): nucleotides 4321-5764 of IKBA
gctactccccgtaccagctcacctggggccgcccaagcactcggatacagcagcagctgggccagctgaccctagaaaacctccagatgcttccagagagcgaggatgaggagagctatgacacggagtcagagttcacagaggatgaggtgagtyccaatgaccttgttcacgggtctgcaaaaagcaatgctctcggacccctagagctcctccttttcctgagggtctcaacataatgaggatctcaaattagggagcataagcagtgtcctaagagtaggtttagggggaggattatggtytggggttttcttttgcttttttgctctttttgaaggagaggatccttaaaggawaacttcagcccaggaagttaattcagattcgggttagagggaacggagtccaagaatacttgcgttatttccagtagcagcccttgccatcaccccagcacctttggcaaagttctggaagtttaacatgcctttctttccccttttagctgccctatgacgactgcgtgcttggaggccagcgcctgacgttatgagctttggaaagtgtctaaaagaccatgkacttgtacatttgtacaaaatcaagagttttatttttctaaaaaaaaagaaaaaaagaaaaaaaaagaaaaaagggtatacttataaccacaccgcacactgcctggcctgaaacattttgctctggtggattagccccgattttgttattcttgtgaactttggaaaggcgccaaggaggatcatcggaatgcagagagaacctcttttaaacggcaccttggtggggcctgggggaaaggttatccctaatttgatgggactcttttatttattgcgcttcttggttgaaccaccatggagtcagtggtggagcccaggtgtatctgggaaatgttagaatcaggtgwgttgttaaacctgtcagtggggtggggttaaaagtcacgacctgtcaaggtttgtgttaccctgctgtaaatactgtacataatgtattttgttggtaattattttggtacttctaagatgtatatttattaaatggatttttacaaacagaattctgatcactgtcttcttcgggcagctgtgggactcctacactgagagtcattcgaaccccaagtggaggtggaggtggagaattgtgtgggagcatttaccacagccaaccacggaactctttcagagaacagcttctcacaccgtctacaccagcctcccggccaggctttgcaggcagccccaggcccagtgcgtgggaggggaggctgttgcaaggtgataggaaacaccagtttcaggcttggggtggcagcaagttggttggcctacagctggaaggctcttcattgtcgcttgctttcatcttcctggtttaaattcagccaggaccttacttctgctttaggaagctt

SEQ ID NO: 63 - DNA, 716 bp, Sus scrofa
tcaggctgtcaccttttatgaaaattttataaagttttgaaaaaagaagaaagaaatctatcatgggttgttgaaagttttatattcagaattaattgtataatgtaaatccaaratacataacatttaaaatctacccatatatagagggatataagtggaagtaccatagctgtaaacacttgagtatagataattattttaacttaatttctcccatwctttttaaagacatgacagcaagtacraraaacaaacaaacaaacaaaaycagagtattgtgcaggtatatcaatagccctcaaggaaagaaacgattccagcattactacaggatgaagtctttgcaacaataaacacaaaaaattgactgaatgacaaaacagaattggattttctgtgtctgacacagaatttcsatcttcaaatagatgcctctgggtatatttttccaaatgttgcccaacaattttattcataaatatcacactttgaaaattcacctgctgtacctyaaaatgataatctaataaaggaaggacagaaaaaatactgcaggatgctcagatagacctcctaggacttaactaaatacatctaacaaattgaatcagaattatcattacttgacagctttgtatttgattacaaataattaccaaaacacccagtaagatcttgcttttcaaattatgtaacattccrttacacactaa

SEQ ID NO: 64 - DNA, 966 bp, Sus scrofa
cacttttttgtgcctgtaggtccatggactgtatcgcacaacaggtgctagttttgagaaggcccagcaagagtttgcaacaggcgtgatgtccaacaaaactgtccagacggcagctgcaaaygcagcttcaactgcagcaactagtgcggctcagaatgctttcaagggtaaccagatttagagagtcttcaaataatacactgttaccttttgactgtacttttttctccagttactgtattctataaatatttttttgttcaaaacacacagtacacacagcacgtatatttcctaatcacttgtgcatgggctaaaaccagaaraacttcgttgtcttattatttacctgacagtttcttaatctttcagtgccccttgcaggaaaaaaaaattacatgctaaataaatattctccatattttttgggggatgaatgttcagcaaattttytcggtggtgacacactgaaatcgacatggcatttaggattaaaaatgcacttagtacttgctgcartcattctttcaagagtcttagacataaggattacacactggagcagtaaagcaatgcttcattccttttctttatttgtattgaaagaaataggacatcagaaacttagggacttttaaattggcttgctttttagcagtttcagtcaccagtgaagagcctatgtgcatttcatagtagataatgtaaattttatctttttattttctttttctagagtaattgatattttgatatcaatctctgatcttgcatgggcaccatgtttcctaaaaaaaytagtattttgggttatgcactgcttctggttgtaggattggggagtttgtagaatcataaaaatgattttctgtaatwgtttcttttaaataaaaatttattggagtgcaatatgaggatataatatacagtgcattatccaaaagaaaaagtagataattgatg

SEQ ID NO: 65 - DNA, 870 bp, Sus scrofa
  misc_feature (1)..(870): nucleotides 121-990 of LEPR
ttacctggaaatttcttcagcttgcctcactactaaatatttatttcctgtaactgtcttttattgcatatgatttgttttattggcttcaaagcatatcctcctctattctgtcgctcttcctgttaaatagattgatytaattctaacccctttaaggaatgaaatttcctaaaatttatcatttcccaaagtgtgttttatagaacattgatttcataaaattgttcttaaaaaagattacatgggtaaataaagtttaggaaaccctacatcactgtatgtccacagtgtagaatcatcttvtatactaaaggtttggagaagccctgaattaaagaaacatttgtgactttgtttcatcctatgttcctcaaacttattttaccaaagaaccttttctcctctaaccatattctttagggcgtatgtgttcctttgcatacattttggaagaagctgcttttatcaatcagaatcatacctatagttgcaagcatatgtatgatgacttgctgtgtcatttttctgatggcagtctgcaaagacttacaaatagcagaaactcttaattatgtcattagatcataatgacttcagctgaaatgaatgtgacagtttacttgcttatagaggagactatcgagagattctctacagcaggccctgtctaaccacaggttaaaattsttaaaagtctttgtggatagaggattagtggacarggattagcaatggggttaagagaaatgattgggaagtgacacattgcagtgagccagtccaaatcttgtcatgaaatggaaataacaagatgactaaatgggggaaaaatgtaattgtaatgtatacatgtaaggataacctgac

SEQ ID NO: 66 - DNA, 1096 bp, Sus scrofa
aatctggctgcgggaacataatagagtgtgcgatgtgcttaaacaggagcacccggaatgggacgatgaacggctgttccagacgagcaggctgatactgataggtgcgcaagaacaactcttctcaataacgctcttctccagggaaaacgaaactgtttctttgcagtttccagaaatrctgggggtatgtggtgcatgtaaaatcacatgcttcatagtaattcaacccytgggcttgattaggaatatcaccgaccttttgtttygatggtaaaaaaggaagacacagaaatcaatagaatatggcaaattaacaaaattgcatttgggttgcttgaaagtttgtgagtagaaagaatttgtgytctaaatytgttaatgttgtgcccataggagaaacrattaagattgtgatcgaagactaygtacaacacctgagtggctaccacttcaaactgaagtttgacccagagctgcttttcaaccagcaattccaataccaaaaccgtattgctgctgagtttaacacrctctaccactggcatccccttctgcctgacgccttccagattgatggccacgagtacaactatcaacagtttctctacaataactctatcttactggaacatggcatcacccaatttgttgaatcatttagcaggcaaattgctggcagggtaagcattattattataaaacgaaacaaagggcttagtcagtaactggaatttctgctgtagaaatgatttttcgtaaacgtattaaaacagtaattatttgctagtagaattcttcccttaaaatgagaagtctaatatataatttcggttatagtaaatgttatcactataatctagatgacagaaatattcttgaacagtttaggtctcagctgggagctgagtcttaccttctttgtacccaagggatgcytttaaaatagaaatcttaaatatacctaaaactcatgttctacaatttcatttcatttccacaggttgctggtggtaggaatcttccagctgcagtacaaaaagtatcaaaggcctcaatcgaccagagcagagagatgagataccagtcttttaaa

SEQ ID NO: 67 - DNA, 765 bp, Sus scrofa
caagagcgtgtcgctgctgggaaggaaccctgctctccaccgccaccctctctctcaggaccctgtgggcyrgggctccacctcctcaccctgagaaagggaaccatgtccaaaatttggatggaccagtgctcccargttttcatcaggtcctggacacagtcgtgaagggcatgcactaaggtgtcctcctgccggaaatggaggaaatcctttcagatcaggacctggagaaggtcaggcagcggctgaggggtgggtccaggcacatgtgaaggcaagagccttgaccttgtctccaaaggtgaggcaacagatgatgctgcaggtgaggacagagaattccttctggatggtcacrggggtccccgcctgggctctcatgcgctgtggggaggagcatgaactcagtagggggcctgccaggagggggaagctggtgcaggatggctgagggggtccagcctcacctcacagaactcctgggtcagctgctccacccggggctccatggagctgcggacgcccagcagcagggctgagcgggtgagtttcttgtgagcyttccagaacagrgagtartcccctagcgagatgtcggggcagtgctgagacgccagcttgtctgtgagtaagggttgggggcgggggttcgaactgaccaaaggaagtcacggacctgaccttccccgcctcctgcagcccctgccccttccttcagaaagagccccaccccttacaggatggtatctggggtcctgccgg

SEQ ID NO: 68 - DNA, 1801 bp, Sus scrofa
tgtaagggaaggttctgccacagttctcttcttgctggtatttcctgctggtgtgagggaaacatattgctcttcccgacatggagtgagtttcatcaagaaataaaaggaaacaaaaaaaatagaagaacaaagaaaaggagttctctgtggcgcggcaggttaaggatctggtgccgtcactgcagcggctatggcctctgctgtggtgcaggtttgatccctggcccgggaacgtccacagactctgggcacagctgaaagacagacagacagacagaaggaaaatgatgggtgggctagagtaggattaactgagcacaggggtgggaggggtgtctgaggatgaccggaggataactgcatgctggtttctgcttccgctgtaaggttacaacctctgggaaaaccatttcgttgctctggccctctttaagataagagggctcctctcctacccagcatacatgttccaactaaaagtagaccttcaagatattctgcactatatagattttgtaaaagtagcttcggtctctcttaatgtgaaaattgcatattgacttaatctcttcccctctctctctccccctccccccttccctcttcccttgcaccccctcactcttcttcttctcctttcccccttcctataaaagctaccacctcatcctgggcaccctggttatatcaacttcagctatgaggtaatttttctctttactaattttgaccattgtttgacttaacaatgccctgggctctgtaaagaatagtgggtggattcttcattcaggatgtttgtcagtcccattttttcagttctcactgccagcttcctagtttaagccctgatgggtcacctcaagcctgcattgccccagaaccctcctacctgcccccccaacccaacccccgactcagtstctcctccgtatacggctgtaaaatgaacaccccctggaggggggacgrcatggtagggcagaaactgaactctggctgamcagagttctatcccggcctggaaaatatggggactcaggtaagatgttatcwacctaaggtccttkccagccagaccactcctggttctaagacgtgcacactctacgtgtctccctygctggtctttcggaaagatgagcgaccaagggggctgtgtgacattctgccgagcaagggaaagtatgagatggctggaaatcaggtttgaggcgcttctcatgcccacacgaaccatgggaccttgggcaaatcattgtctctctctggaactttggtttcttcatctggaaaagggaaatgattataatacccaacaatttaaaatattgattggggagcgaaagagttaagcaacataaaaggtgctttgttcagtttgccttgagcaaggtcgtaattacggtattgctatcaaatgcttattactgtctgaaggagtccctggacctgaggttactcgagtctatacggttaaggaaggaaggaagtgctgacttctttcctcggttcagatgacaacccatgggtatgttgactcctacaagctgaggacaagggttaacaaaaatccgaggaaagattttctgttaaatctgaaaaggttgacatatgtaaccggcaaacgcgtttctaggatgagaaactggtttggcctccttaatatttttgtgacatcagatcaaaagaggttacaattcctgtgaggtcacattaattctctgttttgtttttctcttgcaaagaagagcgggcgctggggagcgagacttactgcttttgtaagctccgtcca

SEQ ID NO: 69 - DNA, 508 bp, Sus scrofa
ttgatgaagctttcttcattcacgagagaaagtcacaatttgataacctccagaaaccacaggagcccatcagaagactacccaaagtcagatgatctctagattgaaggaaagcaggcctgatccttaccagcaaccctgacccttgaaccttgatggtaagaatcctcagaaatctccaggttatgtttgtttgtttgttttggccatgcccacagcttgtgaaaattcctgggccagggtttgaacccrgcatcaacagtgacaatgcrggatccttaacctgttrcaccacaagggaactctaaggacacagggttttgagggcattcgtccactgtgtccctctttgcctggcaaagcaataaagctcttcttttctaccccactcaaaactctgtcctcccagattcaattcggcrtccatgcacagaggccgagttwccccatcagtattggagggaattgttaagcggcttcagggkctttttttttttttttttttttt

SEQ ID NO: 70 - DNA, 1082 bp, Sus scrofa
cccccacggggtggggcagagtctggggctgcagagtcggggtaggggatcagccggagcctgatgggagggcctttctccagctgytggggagatggtatctgaaggccatgacctcggacccggagattcccgggaagaagcccgagtcggtgacccccctgattctcaaggccctggaggggggcgacctggaagcccagataacctttctgtgagtgtcgcctcccgccttcccctccccgcaccaggagggcgggggtctctggggtgtccttctcagccccttgtgtgacacttagccctggacagctctggggggaaccgtcctagaggggacagaccccggatgagaccctgtgggtgggagggscagtgctgggagacccaggcaactgccayrtgccagctgatgcctggcctggaggtggctgacacgccatcgtccctcccccctcccccccgggctaccacggaccccaggctgcctgtggctgctgggccagggggaccggagccggggctgggccgggtctccaaggtgggtgacccccaggcagcatcacacgtggcttctgtgttccaggattgacggtcagtgccaggacgtgacactggtcctaaagaaaaccaaccagcccttcacgttcacggcctgtgagtctcggggccctggccgggggcaggggtgggggcggccagcgagtttctgggacggtcttgcagcctgaggagccctactgctttctgaccctattaaatgccaccctctcctcccactggtccatttgccttratgatatgaaccccygacgccaggcgtgacggatttgccctcggggggactggtttgtcccgagctccagctgggggtcatagctgtgccaggtcaggcccaggagcagagcctctttgagcccccctcccccgctgtcaccgccggttggggtgtgaccacctctccagtgtgggctctcccgggacgtgggggccccacagcctggggggtcggctggggtggaagcccggggcasgtgccccgggagggtcctgggcttaccgggaccgggcccgtctccgca

SEQ ID NO: 71 - DNA, 3680 bp, Sus scrofa
cgccttccaaaacggggtttcttagaacacacagcttagaggtgcagtcagtggggctcgtccgaccgatctggggccatccaaggcctggcgcacgtgcagagcgggaacgcgaggggccggcgtgttcgcggtcgcccggcccgcctgtgcccaggcccctcggaggagcagagggagaagtcggagcttcggagcgagcccggccttgggggaaagagcctctccgctccccccaataacacagagcctacaaaacggcaggctgatgagaacagaagagactttaaaaaaaaaaaaaaatctaaatccactccattactgaaagtgccatttcacaattttagtgggccgttattaggcccactgggtgaaaaaacaaattcttcccacaactgaagtgcatgaaaaagataaacatctaaaagtaaagccatctctcctgtcccggattgagaaggcaggtcagcttgtgtcagctgaaagtagggagttctatttactacttttttttttttttttttaatgtgggagcaatggcaaaacgttggatttccttcgttcctttggcctgtgacaccctttaagcgtcccttgatttgctctaaacagcgcaagtaagcggggtgggggagccgtcaccccgccccagcagaaaggcagtaaaaccattagcgtcgaagggccggtaaacagcccactgtctctaaaggaaaggcggaggtttgcccaaacagccccggcgggggttgcggtgggatatgctaatagtgccgggccactggggccggcctccctcccccaagaacatataaagggccccaaccccagcgcggccgacccaggccgccaggcgtctgcccctgttaattagcagagcaaccgagcagggagttccgcccgcgacgtgcccgcccgcggaggcgccaggccccgggcttctccccgatctgatctatctcgcagctgcccaggtgcaccgcccgcctgtccgcagaagatggacctgatggacggctgccagttctcgccttctgagtacttctacgatggctcctgcatcccatcccccgagggcgagttcggggacgagtttgagccacgagtggctgctttcggggcgcacaaagcagacctgcccggctcagacgaggaagagcacgtgcgagcacctacgggccaccaccaggccggccactgcctcatgtgggcctgcaaagcgtgcaagaggaaatccaccaccatggatcggcggaaggcggccaccatgcgcgagcggagacgcctgaagaaggtcaaccaggcgtttgagacgctcaagaggtgcaccacgactaaccccaaccagaggctgcccaaggtggagatcctcaggaatgccatccgctacattgagagcctgcaggagctgctgagggaacaggtggaaaactaatacagcctgcccaggcagagctgctctgagcccaccagccccacctccagctgctccgacggcatggtaagagaaagctcgggacctcctaggcccttctaatcttttccaaaaaactttacctctcgtttaagccaggtgtagcaaccgaatattctgatagttggctgtgggggtgaggaggcagttgccctaagagagatgccccatttagacagacgccaggaaaccgctgctgaagagcataatactttgcctcccaagttctaggtgaacgttgccgggggaggttctcatgtgagacgggtggctgtgaatgatcagaggttttctccattactcactttacttccgatatataccccccgtggaccccaccgtacactaacgtttaaagrcaactgacggaggctccccatagcgcatggtttctaactctaggcagaaatgggatgaaacacccgcatggccccggttgctgctctgctgaggcctggctggaagatgttgatgcattcttttcagagggtgtctgctctaacgctgccaggttttaatgtgtttttgccctgggaaagtgttctctctccgaattagtgtggcttccttccaccccaatccattttgcatggttaacccagtgcacgttgctgccgaattccaccccgcccctctccttttctcccagtacagtgactgacctcagcgcccttgccatttggggaggcgagcccttcctaaatcaaggcagtgaaggtgactgagagtsgtcaactttcgaagctggagggcaagcactgcctcaccctccatcagagcacctttcgccaagacctgaaaacaaatgccttcttttgtgtcttttattatagcctgaatgcaacagccctgtctggtccmgaaagaacagcagttttgacagtatctactgtccggatgtaccaaatggtaagaacgaacgcctttagaggaggttaaagaccagttcaacttcacagttcagcccatcaaatcagtctgtccttccaggcagttatctggaggaaaagagaatggtttttacagggactttttggcggagcaaaataaacatctgctcaaaattcccctcagagatactcccatgcacacacacaggcacatgagcttttgcttaaaacattaccgagggtgcttttccactccccacctgcacccccatgaattgctagatatatatgttgcaaatttctacactggggtctctgtgaccacctgacctctgggtttcaaaggagctgacctgcagttcaaagggcaacgtaagcaagtctacctattgggtttttttttttttaacgtttttttttccccttgtatctttagtatatgccacggataaaagctccttatccagcctggattgcttatccagcatagtggatcggatcagcaactccgagcaacctggactgcctctccaggacccagcctctctctctccagttgccagcaccgattctcagcctgcaactccaggggcctctagttccagacttatctatcacgtgctatgaactaaaaatctagtctagaccatttctgccaggagtgcctattacacaggaggaaggaggcccaaaaggcccaaaagcaagacaacctgtatataaacattttttttcagttgtaaatttgtaaatactatcttgccactttataagaaagtgtatttaactaaaaagtcactattgcaattaattctttatttcttcttcttttcctttgtcttggcattaaatatatagttccaatgatattatttcttataggggcaattcatccaagggtagctcgttgcaatgcttaacttatactttttataatattgcttatcaaaatattacctctgtttagagctttatttttttcccctttaaaaatattagaacaaatactagaactggaaatcaagttatagggagttttaaatatatttaacttttttgcttctctttaatcctttggttatattgtgttaagtaaaaatataacatactgcctaatggtatatattttgatcttataagaaatgcatctttttaatgtaagcacaaaatagtactttgtggatgatttcaagatgtaagagattttggaaattccaccataaataaaattgtttaaatgaagaatcatttgatttatgattttgttaaaagaacctctaatagcattggcagtgattgatacgtatctttgagct

SEQ ID NO: 72 - DNA, 491 bp, Sus scrofa
atgtctccatttcttttgaagacakatgagayctgagaaaaggtgatgctccctgatgtagtgggctcagcagtgtctgagcagaactctcaggacaacctgtctragacrtccaacktgatagcaaagctgagacaaaatgccacctgctgactcaccgcaggagacagcaagctggtaygatatcattagagaaataacaacacacaacaatagcgggcacttgttgggcacttcctctgaaggcagtrtgccaggagcgatcttcacatcttcattcaatccccaggacaacctgttatcagaggcttcacgatatccgccttcatccataaaataaagtatacaacatgctcagcactgatgggacagagagaaggtagggtcccacatgtctagtctttaattccacatcatgtgacaccaggctacaagaaaaatctgaaggcagaaggcagatctggagatctagctggccaggtttcctggaa

SEQ ID NO: 73 - DNA, 553 bp, Sus scrofa
cacactcacgcctcagaccccatcgctgtgcagtgacatcggcattgatgrtggccggtcacycctgcaccagcyggcccctcccatttctccagggacaagawgtagrtttgaggttcctcgtctgacttcagcctccccctrtttcctcactcrctgtcccgtcccttctccgtccctgtttctggaagycgccytccatgaccaggacactcagattcttacttctgaggattatactgaaacccttgccactcccttcctgtgkcctgttcatcactgcgtcacctgaccaccccagcaccctctccctrcgcygtgcagacaccctgttttgcrctgtccccagcagctggcaggaggaggtctcatcacagagcagccctggcccaccgagtccccacctrcccaagaacacggccctcactctgcaactggccacgccgctgctcaccacccttgcctygtgccaggacccgagagmgcactggtccttgtgagtggygtcctctgccttcaagcatcagttcatccatctccccacacacacgag

SEQ ID NO: 74 - DNA, 554 bp, Sus scrofa
  modified_base (12): a, g, c, t, unknown or other
tttctactacyngscatcatttcccttccgactgtaggatttaactttaacatttccttkgtacaggtctgctggtgatgaacgctttctgcmtatttatgcctgaggaatctttrttttgtcttcattattgaaagatattcctgctctgtatagaattctacactaatagacttygctttcagtaggtacttcggaagatgctgctccactgtcttctggcttctttgtttcctatgagaattctgccatctttatctttgcttctctgkatggaatgtgtcatttttttcctcagactgctaataatatattctctttatcactgggttccagaaatttcattctgatgtgtctkggtgtaattttctttacatattgtgtgcttggggatganctgtgattcttagatctatttatagtttttataaaactttggaaaattcagcaaattcagcaattaatcttctttttttytcctgttgttccttctccccattctccttcagaaccgcaattatatgtacatttaggsccacsctgaagstatccta

SEQ ID NO: 75 - DNA, 596 bp, Sus scrofa
caaaacttctaacrtgcagaaaaagcggkagartttacagtgataaacacttgcattaaagaaaactaaagatctgaaataaacaacctaactttayacctcaaataactagaaaaagaggaataaactaagcccaacattactggaaggaaggaaataataaagattatttaagagagmagaaataaatgaaattgagattagaaaacaatagaaaatatcagcaaaattaagagctagtttttcaaaagattaaaaaaaaatgaatctttagagtaagaaaaagagaaaagatgagtaaattaaactataactgaaagaggagatgttaccaactgacactgcagaaatacaaaggatcccaagagactactatgaacaattatgggccaaaaaaattgggtagcttggaagaaatggataaattyctagaaacatagatcccaccaagaggcaaatatgacaaaaragaaaatctgaacagaccaataatgagtaaggagattcaaycaattaaaaaaaaaaactcgctaatgtaaagcccaaaactaaatggtttcactgctgaattctaccaaatwtttaaagagttttaa

SEQ ID NO: 76 - DNA, 673 bp, Sus scrofa
ctgctccctccacaccgtctctttctttgcgggtcggtgggttcctcttagggacccagayaacttgctcctccctacctctcatcyctgaaacccgaatttctgtccctggcctggcagccgtccacccccagccctccyagctaaggcgtttaacatgcctgcccgcagtatggtggtctagaaactgcccaggagccagggggttccctgcaaattctgtcccaggctgtggccccagctgggtgggggaaagggacctgtaagctgcaagataaggggacctcaggtgtggggagttgttgatgttttcacctacaagtgtacctgtttgctggcttgtgtgtattcccacctgtctgcctgggactctgaaatagtctggcctggggtttctaaccctaccccccaaggtgaggcctccaggttggggtgggaagtctaatcaggtttccccattcctgaaaatggggctgtagtgagttgcagccatttgctcaaggccttctcaggagtgggaggcagcgggaatcagaggaaacagtgaatcaaagagactccaaagagggcccatctacactgacagaagaactagcagcccgagagtaaatccaacccctgttaatcctgtttgatgtctcttggartgatgcaagtcattgagacactcact

SEQ ID NO: 77 - DNA, 511 bp, Sus scrofa
  modified_base (360): a, g, c, t, unknown or other
gcggtcggggttgggtagcacatcctggcctcttcctgttggccccacaggctgagggagatgtcacttcctgctgttgggaggtgaccggcaggatggasgggagcaggaaggggtggccgtgarccagcactaaccagcctgggccatttcctcaccccggaaagagggaaggagagagagagggaagacyggtgtttgggagtctcctgtggccctcacacccagagrcagtgggtgctcagacccctgctcccacgggcactgatctggcacaggcaggcgttcagaaaaaaaaaactttcaaaacaatcctatgcactggcccctgcacgtcacagtttccaatggccttctcanccccggcctragggctttcctgccatccctctgactgtaggacagagttgggggattattgctgccaccggaaaatgaggaanctgaggcccagtgaggttgcaatgancagtctgccctggatygtacagcaaattantaccaggacttg

SEQ ID NO: 78 - DNA, 484 bp, Sus scrofa
  modified_base (9): a, g, c, t, unknown or other
tgggttatnggctgttttgattcyragcgaaaatttgagggaaacacaaagatgaagtgaagctcttaatttactttttcactttctatacttcctttaaaaatgacttcaataccctagyatttttccatatgaacgtttctatttttcatgattattaaagtggtacaaaattggaacaactgcaaaatccattggtaagtgattgattgtgatatagtaatgcaatctttatgaagattaaaatgctttattaccataaggagatgttcataatytattttgaagtgaaaaaagcaggtggcacaatagaatatatttgatgtgaaaataaccattaacagtggtggcatgagtgaaaggaaggttgtagagggactttccaagggacctaatcttctatgcttgraaaatctgtagacaccttaagtgcaygtaactaacacctggaggttgatctttacactgrggsnssaaaraaaaa

SEQ ID NO: 79 - DNA, 1500 bp, Sus scrofa
  misc_feature (1)..(1500): nucleotides 1501-3000 of PRKAG_STS3
ttaacaaaatcgatactatgtgtgtctgtacacttatgactttggagtagaaacactgggttggtttcccacaccttggagtgcttggggaggggtcacctcagyacctctggccaccagcagccttagatctggaacaaatgtgcagacaaggatctcgtggagggcatgccaggacgtgggagaggcagacagcaggctcatgtagaggcaggcccgggaggcgcccggtggaagaaccctggctggcaggggacctctgaggcgcagggaacgattcaccctcaactgttctctccggcgctcagatcaagaaggccttctttgctttggtggccaacggcrtccgagcggcacctttgtgggacagcaagaagcagagcttcgtgggtgaggaggggctggggaggcagaggtggyggggaagggaataggggraccttgtggggtgattctagggccgagctctgacayaccacaggcttsaaccaagcaggggcctggcctggrgrrscggagggagcatytgaccccggtctccyggtggccrgctgggagatctcaactgtaggagagctgtgaccagctgacccctccagctctactaccccaaggtccctgtcgcaggtgctaagtaagaagaggacaggcggaggaaggaagtcagaaaatagaagaagcagggcaggaaggagagaaatgacaggggaagcataagagggacaaccccatttstcaggcacgggaggggctgccctcctgtcctcttttggccaccctcagtaaaaggatgtgggcagggtggggggaggggcccgggctgacccccattgctccccccyyttgccccccsyacagggatgctgaccatcacagacttcatcttggtgctgcaccgctattacaggtcccccctggtgaggagtggtctgggggtcctggaacacccatctgggctggggtggaaggagttcaggggaccctcgcctgactttgggagttccgttgctgtctttaggtccagatctacgagattgaagaacataagattgagacctggaggggtgagcaggcgaggggacsgkcgaaggggctgagggtgtgtgggtgaggatgrggccaaggacctcagggagagcatgcgcagtggaggtttcctggaggaagcgggaggagggtgatcgggagcccaggggatctaagggagggagacagtctgggggtggccacgtgaggcggggtggtcggcccctttgtgctgattctggcttttcctgcagagatctaccttcaaggctgcttcaagcctctggtctccatctctcccaatgacaggtgagcttccccagccrcccactcgagcctccttgccccgcacagaccccttctccagctcatcggttctaagctcatggactcatcgtccgtggactgcagatgcggcagctttgacaccctgtcctcctctccaggggggctgggatgaaggggctctctttc

SEQ ID NO: 80 - DNA, 1077 bp, Sus scrofa
acggacattcctgacctccactctttggcctgagggctgtgaccaagggacagyrggccaccmggtggaacyacaacagccccagayctcccctgcgaagggagtccaggtcctggggtcctaagggaccccccctgccctgagcagccaatcaggccacgtgcacacggctaacctggggctcctcccttcccctctgcccgggattcgattctggagarcacctgcaagcagcggtcccccccaggagacagacgggggcggaagaagcctgcacagcaggacgtgtctggggtgaatyrgggactccaagactgggaagaaggctgttctcygtgcctagaacacatctaggagcactgggggctcagaaaaggcaygccctggscrtgraactcctcarcacttactgctcaaatgttacactctggtacaaagggaatcagaarctcaagaactcccagggctgatacatargcaatgatcttgyggcaaactgtcycctcctatgcggtttgaccttgtcctcctgggcgccccctgtcagccctttggcrgtrgggttgggcatgagscctgccacgggtggccaagaagcygtgggaaaggtgccctgccttggagtctgggtttgctcatcaatgaaatgagccaacatggaccttgagggtgtcgaggtgaggttgcysaggtgaggtggggcatcrtaggacctrcaccgggtgtggggcccaragccaytcacgaggcccttcccgggccctgctagagacacrcccacactacctggggtctcggcctctagatttggagcaagaccaaccctggtggcagyggcagccgctccagggtcacgcccctccagtcttccagagcccgctctggggacccccacccccgggaaagtggctgccaaaccattgctcagtctcctcaggactggccgatgggggctgcgctcgctcaggtcccctcggtgggcgcgtgtgttggggatgggcggggtttccctccacgtgtttgtgagccacacggactagcgccgggatgcctggcacaggtgccttggggaggccc

SEQ ID NO: 81 - DNA, 2401 bp, Sus scrofa
ccccagccctttttccaggtcagcscagggaaaaaacatgttctctgtccctggttatactgtttagaaacatcacctccctcggcgwwactaaaacttgggggttgcaatttattccttgcttctttgtatttcktaccacattgagagagctctaggttttcatccgcagattcccaaaccttcgcagaggagctgtttcacaggaccgtgattcaagtttactctacttttccatcatttatttggtcatatgtttaaatgaagaaagaaaggaatgaagatacctgaatgaaatgagtatttgttttcttaccagcaggactgaatacaaatgaagagaagaaaaatacgcacatttaggacttgggcagaggttttatccacgctctccttgtggttatttcccatattcagaaggcrcrggwgwggattygtctgtatggtcctaaattgaaccacagtggtcaaatccctccactttctgctccttggattcttcgtttgtgtactaagaaaatggggaggcagtctctaagagattgctacagtgggactcaactctaaaagttgtacagacttgctaaggaggatgaaattagtagcactttgcactgtgaggatggkacctagagctccccagagaagggctgaaggtctgaagttggtgccaggaacgtctcgaagacaggtatactgtcaacattcaagcctcaccctgtggaaccacgccctggcctgggccaatctgctcccagaagcagggagggcaggaggctgggggggcataaaaggaagagcagagccagcrgccacctacatttgcttctgacacaaccgtgttcactagcaactgmacaaacagacaacatggtgcatctgtctgctgaggagaaggaggccgtcctcggcctgtggggcaaagtgaatgtggacgaagttggtggtgaggccctgggcaggttggtatccagggcttcaggagagggagygggaggtgggcaggtggggacagagccacccctgcctttctgacaggtgctgactccctcgggccttgcgctcttttcacccctcaggctgctggttgtctacccctggactcagaggttcttcgagtcctttggggacctgtccaatgccgatgccgtcatgggcaatcccaaggtgaaggcccacggcaagaaggtgctccagtccttcagtgacggcctgaaacatctcgacaacctcaagggcacctttgctaagctgagygagctgcactgtgaccagctgcacgtggatcctgagaacttcagggtgagtctgggggaccctcaygttctccttctgctccttggtcatggctgagctcgtgtcatgaagagagggcygaaaggcaggatgccgtttagaatgaaaaggaagcattctggttacatmgctggggactctgcaggaccactgartttcttttccctcttcttattcacagccatcatttcctcttactctctcttgttctcckctgttttgtttkgtttkgtttkgktttgttttgtttttttttttttcctctgcgatgtcttctctcttttaagttaaactttttgagtgttttgttaaaaaaaaaaaaactttcttcttttaagccactttaaaaaatgttatctaacagttgccccttattctttccttttgaggcaagaaggataaaatgtttattgcttcctggcatggttctaaagcataaaaaggataacataaattcaagactaaggtagaaagagagaaacattyctagctgtcattcaggttgccatgggtggattcacatcaatagtaacgtctacacggcagctcactttctgtttatcttctaggggctcagcttgggatgagactgaaatactcctgggtctaagctggtcctctactaatcaggcccttcctttttatctctctcccacagctcctgggcaaygtgatagtggttgttctggctcgccgccttggccatgacttcaacccggatgtgcaggctgcttttcagaaggtggtggctggtgtggccaatgccctggcccacaagtaccactaagttccccttcctgatttccaggaggagccctttttcctctgaacccaaaaactggatatggaaatattatgaagagttttgagcatctggcctctgygtaataaaaacatttattttcattgcactggttttttaaaattatttcagtgtctctcactcagatgggcatatgggaatgaaaggtattataaaaaaatrtaaataaatgaggggctaattgagactttaagaaagctctctgtaccttggaccccatgaaagragtggttgtaaagcacgcatgtgagtgggaatacaccttatacctcttcctttcct

SEQ ID NO: 82 - DNA, 2053 bp, Sus scrofa
ggaatttcgatggctctagtacttttcagtctggaggctccaacagtgacatgtatcttgaccctgctgccatgtttcgggaccctttccgcaaggaccccaacaagctggtgttctgtgaggtcttcaagtacaaccgaaagcctgcaggtgggtagaggataggtctgggtgcccccaccccaatccatgaattggaaaggcggggaagaggctattccaaaattggtactgagaagaccagcctgtcttagaactgtatgtgaagtctgtattcgcttgggcctcatctctccatctagaacatccttgttcaaagaggttgcgtggcccatcctcgcattattggtggcataaaacaacattcaatttctctctctctctctctctctcttcttttttttttttgctttttagggctgcacccgtggcatatggaggttctcaggctaggggtccaatcagagctacagctgccagccacagccatagcaacgcaggatccaagccccgtctgtgacctacatctgtgacctacaccatagctcacagcagtgccggatccttaacccattgagcaaggccaggtattgaacccacaacctcatggttcttagtcggattcatttccgctacaccacggcgggaactccaacattagattcttctaaatctgcttgccgttagtcctctgtggcacagccctcctgcctgcctgtagattttaagaacacagattctaaagttagaatgttggatttgaattctagttgtgccacttccttgctrtgaacttgggcaagttacttttcacctctactcctcacttccctgattttaaaatggggaaattaataaaatccatcttagaaagttgtttrtggggcgaactgagttgatgagtgcggataattgagaaggctcgtacaccacgcactrgttgccttttcattccacacagcgcgtttcctttctctgcccacggactaaaatgaggacatccggcttcaccaataacatacgtttgccttgagggttgctggagccccgcccctttgstgtttgtctagcacacccattgctcccagttctacttcgttcgtctttttaaatttgccatgctctgccagtgagactttttggcaagtcacttagtgactctggaccttattacttgtctttgtttggagaggcattatcatattgtggtaaataacttgagctcggaagttgagacaagcagagtttcctggctctgctgcttttgctgtgaccygggcaggctacctgccatctctgagcctcattttctcatttgtaaaaacggagactcgtaatccccacctcagggttgttaggaagactcaagattataggttttctttgataaaatatgaaggtcataaaatggttcctgggacatagtggaatagttatagtaatgagtccataaagtaattctagcttagaaatagtttcagttgagttcattgcattttactggggtttggagggggtttgtttgcttttgcaccagccaatactatagaactttgtagtggtttaccttctcaccttgaatgtaaggctcttcttctctcagtcaaaattgtagatttrgctctaaatgcctgagaaactaggaatagatggaaagcgagargcagttctctagtgaatagagagtttaagtgcttgacatttggcgtttgggagggggccagcttgcagataaagtgaaaacgtgctctattctggatccactgccgtgtgactttgttgtaacccagtttagttataaataaagccttttgagagtctccctacagatacataattatagcttctctcattgtccrcttttacaccagcggggactgaaagcttacagtgcagcyracactcatcatctcaggtgtgyggattcctggaaaccacctcaggcgcatgtgcattcggtacatttgcccatgcgtctctaataagtgtgttggctctttctttttcctagagaccaacttaaggcacacctgtaaacggataatggacatgg

SEQ ID NO: 83 - DNA, 840 bp, Sus scrofa
  misc_feature (1)..(840): nucleotides 1861-2700 of IL2R
ctcctgctccgacttttaaatgaattgccttccggacacaaagaagtggcgggctgccgagctgacggggctcatcattcatggtcgcactccttgccatgtgtgatgccrtcaggcggmcggccrgaatggagtccctctgggggagactmatacacaaattccatcagaaaatctcagttgagaaatacatcrgaggccatggtcrtctggacaggaagcccgactgacaggctggcrctgggctgaccatgaacagrcacaaccaagagcccccccaagtgcttatctttatgaccccagtaccccaatattggctggcaggtcgtgggctctaaaaacaccttctttttgaatgaatacacgaagggcattttctcttgtacacatttgtgaagttattttcttcatttcacccctactcccyagcctccccgaggatccccgtaaaacaagttactcctagacctgaagaacagaaggaacgaaaaaccacagaaactcagggccagatgcagcccccgaaccaagctaatcttccaggtaagaggcaaaccatccccctgcaggtagaggagatgctgccctcaggaggcgtctgtcctctttcctaaggatgccrtgtggacaagaagcgggtttgcargctggtcactcagcacatcaagccactctgacagcagccaacctgggttcagggcttcctctgggtttgggaaccagccaaacrgaattcaactccattcaatccaactcaactcactkcacttcaccaagcacatctggggactacctgcggccaggagatgcaggcctgaccagacagtggtccccgtcctc

SEQ ID NO: 84 - DNA, 718 bp, Sus scrofa
  modified_base (129): a, g, c, t, unknown or other
ctccctcmacctacmawhwttcctaaggtttgcagaggagctgccatagagctcaaaacayggtctacagacaagcatcttctccatccctyctcatcttckcacrggccgyttgacaacatctctwgnggakgkggdggrgcngncscyvcncvmsckgkdwrwcsmccyygcgtyyacscmaagmgctctgactctggagttctagtcctcgcgggaccttaggaagttcacggtcaatactcygcccttgggctcagwcactaagakgatctccgggtaaagagatagacagtagctccatgcctgatttaggaaaactgtccgtacagacagttgtaattcatttcctttcagagacaaatcctgctctcttcctagttcctgaagtcattaaaatcaaaagctctcagaaacgtcccagcatttgctaagtccacgctgggggaggatgggcagagccgtgttcagcarcgtttgacagcaacacccacttattttcattcagtatccataggcatatatcatgcacctggtataggyctctctctcagcactggagatacagcaaagaaaacgctattcctgcyccatggagcttgnttcagaaaaataractcaaaacctttcaaatggagctgccgctgggccaagtcaaaataagtaaaagaaatccgtgagaaacccttcagttatattaagaagaaatagcttgatg

SEQ ID NO: 85 - DNA, 1585 bp, Sus scrofa
  misc_feature (1)..(1585): nucleotides 15001-16585 of mtDNA
ggatacatctcagtagccatagcagtagtataaccaaaaaccaccaacataccccccaaataaatcaaaaacgccattaaacctaaaaaagacccaccaaaattcaatacaataccacaaccaactccaccacttacaatcaacccaagtccaccataaataggagagggcttagaagaaaaaccaacaaacccaataacaaaaatagtacttaaaataaatgcaatatacattgtcattattctcacatggaatttaaccacgaccaatgacatgaaaaatcatcgttgtacttcaactacaagaaccttaatgaccaacatccgaaaatcacacccactaataaaaattatcaacaacgcattcattgacctcccagccccctcaaacatctcatcatgatgaaacttcggttccctcttaggcatctgcctaatcttgcaaatcctaacaggcctgttcttagcaatacattacacatcagacacaacaacagctttctcatcagttacacacatttgtcgagacgtaaattacggatgagttattcgctatctacatgcaaacggagcatccatattctttatttgcctattcatccacgtaggccgaggtctatactacggatcctatatattcctagaaacatgaaacattggagtagtcctactatttaccgttatagcaacagccttcataggctacgtcctgccctgaggacaaatatcattctgaggagctacggtcatcacaaatctactatcagctatcccttatatcggaacagacctcgtagaatgaatctgagggggcttttccgtcgacaaagcaaccctcacacgattcttcgccttccactttatcctgccattcatcattaccgccctcgcagccgtacatctcctattcctgcacgaaaccggatccaacaaccctaccggaatctcatcagacatagacaaaattccatttcacccatactacactattaaagacattctaggagccttatttataatactaatcctactaatccttgtactattctcaccagacctactaggagacccagacaactacaccccagcaaacccactaaacaccccaccccatattaaaccagaatgatatttcttattcgcctacgctattctacgttcaattcctaataaactaggtggagtgttggccctagtagcctccatcctaatcctaattttaatgcccatactacacacatccaaacaacgaggcataatatttcgaccactaagtcaatgcctattctgaatactagtagcagacctcattacactaacatgaattggaggacaacccgtagaacacccgttcatcatcatcggccaactagcctccatcttatacttcctaatcattctagtattgataccaatcactagcatcatcgaaaacaacctattaaaatgaagagtcttcgtagtatataaaataccctggtcttgtaaaccagaaaaggagggccacccctccccaagactcaaggaaggagactaactccgccatcagcacccaaagctgaaattctaactaaattattccctgc

(19) United States
(12) Patent Application Publication
(10) Pub. No.: US 2005/0272057 A1
(43) Pub. Date: Dec. 8, 2005

(54) Small segments of DNA determine animal identity and source

(76) Inventors: Mitchell Sam Abrahamsen, Hamlake, MN (US); Wadija Freije, Forest Park, IL (US)

Correspondence Address: BARNES & THORNBURG, P.O. BOX 2786, CHICAGO, IL, 60690-2786, US

(21) Appl. No.: 11/041,776

(22) Filed: Jan. 24, 2005

Related U.S. Application Data
(60) Provisional application No. 60/538,791, filed on Jan. 23, 2004.
(60) Provisional application No. 60/539,728, filed on Jan. 26, 2004.

(51) Int. Cl. C12Q 1/68; G06F 19/00; G01N 33/48; G01N 33/50

(52) U.S. Cl. 435/6; 702/20

(57) ABSTRACT

Methods and compositions to trace an animal to a country or a farm of origin or to identify an animal based on SNPTrack analyses are described. Methods of developing SNPTracks that include a plurality of markers with one or more SNPs and SNPTrack analysis kits are described.

This application claims priority to U.S. Ser. No. 60/538,791 filed Jan. 23, 2004 and U.S. Ser. No. 60/539,728 filed Jan. 26, 2004.

Short segments of mitochondrial, autosomal, X, and Y chromosomal DNA, are used to identify lineage of individual animals (identity), and to trace their country or farm of origin (traceability).

### BACKGROUND

There are many reasons for determining the identity and source of animals bred as food for humans. The ability to trace back a food sample to the farm or country of origin offers improved quality control, safer food, and can demand a higher price. The ability to rapidly trace lineage back to the sire and dam better localizes the cause of any problem and gives an opportunity to take preventive measures, for example, to minimize the unwanted distribution of contaminated meat. The ability to rapidly trace lineage back to the sire and dam also provides useful information on meat quality in genetic selection decisions to improve meat quality. Human populations may also be monitored with respect to immigration and forensic purposes. Forensic applications include immigration documentation, criminal trials, adoption disputes, and paternity testing.

Pedigrees are cumbersome to analyze directly and have problems arising from the nature of breeding programs, e.g., because commercial pigs are sired by AI (artificial insemination), the same boar is mated to sows on several different farms. In those situations, the farm can only be tracked via the dam (mother of the slaughtered pig). Moreover, parents may be dead or unavailable for genetic testing.

For example, in swine demonstration of the sire line is problematic. A further complication is that, in order to maximize fertility, the semen from two or more boars may be mixed together for use on commercial farms. Piglets from the same litter may therefore have different genetic sires. To overcome these complications, DNA genotyping requires a large number of markers that are developed and characterized specifically in a specific breeding population. Unfortunately, as the numbers of markers increases, costs of testing also generally increase.

Rapid tracing to the farm of origin of a cattle infected with Mad Cow Disease is necessary to identify and limit the spread of Mad Cow Disease-infected beef in human food chain. Similar investigation is necessary to limit the spread of Scrapie-infected sheep in the human food chain. Current methods of tracing a contaminated meat sample or an infected animal to its country or farm of origin rely on time consuming and laborious procedures, some of which are discussed herein.

Determining the lineage and source of individual mammals previously relied on the use of restriction fragment length polymorphisms (RFLPs), variable number of tandem repeats (VNTRs), and microsatellite markers. All these known DNA sequence methods have the same disadvantages. They are costly to execute and require highly specialized laboratory settings. In addition, because of their repetitive nature, VNTRs and microsatellite repeats can be affected by errors in the replication process that result in novel alleles that are non-informative.

### SUMMARY

New methods and compositions are provided to determine lineage and trace the source of animals, in particular mammals used for food. For use in forensics and food security, the tests are robust, simple to perform, and have sufficient power to identify closely related individuals. Single nucleotide polymorphisms (SNPs) are abundant and simple to analyze.

Short segments of mitochondrial, autosomal, X, and Y chromosomal DNA, are used to identify lineage of individual mammals (identity), and to trace their country or farm of origin (traceability). A DNA test for identification uses genetic information to uniquely determine the identity of each animal and to trace the animal to its country or farm of origin.

To minimize costs and maximize the efficiency of the number of genetic markers needed, short segments of mitochondrial, autosomal, X and Y DNA are used to determine the genetic origin of humans or domestic farm mammals. Genetic origins include family, country, or farm of origin, and lineage. Single nucleotide polymorphisms (SNPs) and/or insertions/deletions in mitochondrial DNA (mtDNA), sex chromosomes, and autosomal loci are useful. Short segments that contain one or more SNPs which map to the mitochondria, the non-recombining portion of the Y chromosome, autosomes, or in any region in the genome are referred to herein as “SNPTracks”. Multiple SNPTracks are aspects of the disclosure. The information content of SNPs and ins/dels which map within 0.2 to 10 kb fragments on mitochondria, autosomes or the sex chromosomes, is combined to generate informative SNPTracks. Each SNPTrack frequency is determined in a reference population of mammals, e.g. humans from different ethnic groups or cattle and pigs from various origins. Frequencies of SNPTracks vary in highly inbred populations and therefore should be determined from an experimental population.

The methods disclosed herein make use of the stability and polymorphisms in short DNA sequences and the minimal amount of DNA needed to reduce the cost and facilitate automated and portable detection of markers, e.g., on a farm instead of in a laboratory.

A method for identification of an animal includes the steps of obtaining a sample from the animal or from a processed product of the animal; performing single nucleotide polymorphism (SNP) analysis that includes markers, such that the markers include one or more SNPs; generating SNPTracks of the animal such that the SNPTracks contain one or more markers with one or more SNPs; and comparing the SNPTracks of the animal to a database that includes pre-existing SNPTracks to identify the animal. The animal is a farm animal, which includes animals such as cow, pig, sheep, and poultry. The animal can also be a mammal and the mammal may be a human.

The markers are designed and developed from autosomes, sex chromosomes, and mitochondrial DNA, wherein, if there are more than one SNP per marker, they are are present within a nucleotide region of 0.2 to about 10 kb.

The various swine markers are selected from the group that includes markers designated ACY-STS7; COX2; EG-STS7; GALT; IKBA; LEPR; P450-STS18; PBE3; PBE42; PBE43; PBE 57; PBE59; PBE 64; PBE 73; PBE 84; PBE132; PBE137; PRKAG-STS3; RYRA-STS6; SCAMP; VAN-STS1; WSCR-STS1; BG; AMG; CTSL; PBE112; and MYF5. The SNP positions of these markers are designated as follows: ACY-STS7 (245 G/C 421 C/T); COX2 (368 C/T 533 G/A 939 C/T); EG-STS7 (774 G/A 805 G/A 817 G/A); GALT (478 G/A 758 C/G 866 C/G); IKBA (4476 C/T 4679 T/A 4904 G/T); LEPR (426 A/C/G 810 G/C); P450-STS18 (71 C/T 138 G/A 361 G/A); PBE3 (115 G/A 192 A/T 555 T/G); PBE42 (111 G/A 118 T/G 181 C/T); PBE43 (314 C/T 471 C/A 524 C/T); PBE 57 (75 C/T 109 G/A 197 C/T 268 T/G); PBE59 (276 C/T 494 C/T); PBE 64 (115 G/A 419 C/G 515 C/T); PBE 73 (93 A/C 116 A/G 177 C/T 477 C/T); PBE 84 (14 A/G 97 T/C 428 T/C); PBE132 (102 C/G 127 A/G 193 C/T 371 A/G); PBE137 (121 C/T 278 C/T 409 A/G); PRKAG-STS3 (1845 G/A 1938 G/A 2050 G/A); RYRA-STS6 (402 A/C 408 C/G 567 A/C); SCAMP (184 C/T 389 G/A 516 C/T 582 G/A 939 A/T); VAN-STS1 (889 C/T 950 C/T 1009 G/A 1065 C/T); WSCR-STS1 (411 C/T 599 C/T); BG (1257 C/T 1323 C/T 1425 C/A 1966 C/T); AMG (907 A/C 975 A/G 1467 A/G); LCN (87 C/T 373 G/C 402 C/T); CTSL (252 A/G 272 A/G); PBE112 (61 C/T 87 C/T); and MYF5 (1833 A/G 2204 C/G 2335 A/C).

The various markers are also selected from the group that includes swine mitochondrial markers designated at positions 15543 (C/T), 15558 (A/T), 15615 (C/T), 15616 (C/T), 15675 (C/T), 15714 (C/T), 15840 (C/T), and 16127 (G/A).

A system for identifying an animal from which a sample is derived, the system includes the steps of: obtaining a sample from the animal or from a processed product of the animal; performing single nucleotide polymorphism tracking (SNPTrack) analysis of the sample with a plurality of markers, wherein the plurality of markers include one or more SNPs; storing one or more SNPTracks of the sample in a computer system; comparing the one or more SNPTracks of the sample with known SNPTracks in a database; and identifying the animal to a particular location.

A computer system for identifying a sample, the computer system includes: a software module comprising instructions operative to provide a searchable database that includes SNPTracks obtained from animals, the SNPTracks include one or more markers, wherein the markers include one or more SNPs; a software module that includes instructions operative to provide an algorithm to determine exclusion probabilities; and a software module that includes instructions operative to provide an interface to accept and compare a query SNPTrack with the plurality of SNPTracks in the database.

A method of developing a database for identifying an animal or a product sample derived from the animal, the method includes the steps of: performing SNPTrack analysis of a plurality of samples obtained from a plurality of animals from one or more sources with a plurality of markers, wherein the plurality of markers includes one or more SNPs; obtaining and storing the SNPTracks of the plurality of samples in a database, wherein the database is searchable; performing SNPTrack analysis of the product sample; comparing one or more SNPTracks of the product sample with the SNPTracks of the plurality of the samples stored in the database; and identifying the product sample to a location. The database further includes data selected from the group that includes production farm, farm of origin, retail outlet, whole saler, breeding record, animal identification, offspring data, sibling data, and lineage data. The sources are selected from the group that includes farm of origin, production farm, processing center, retail outlet, distribution center, and whole saler. The SNPTracks of the plurality of the samples stored in the database are associated with an animal identification system.

The SNPTrack analysis is performed on the plurality of samples between birth and slaughter of the plurality of the animals. The database may have limited access to an authorized user.

A method of obtaining a SNPTrack of a sample, the method includes the steps of: selecting a first marker such that the first marker has one or more SNPs within a 0.2 to about 10 kb region in the genome; selecting a second marker such that the second marker has one or more SNPs within a 0.2 to about 10 kb region in the genome; performing SNPTrack analysis of the sample with the first and second markers; and obtaining the SNPTrack of the sample. A method of obtaining a SNPTrack of a sample further includes the steps of performing a SNPTrack analysis with a plurality of markers and obtaining the SNPTrack for the plurality of markers.

A nucleotide segment from swine genome that includes a plurality of SNPs, wherein the segment is amplified with primers listed in TABLE 3. The nucleotide segment is selected from the group that includes autosomes, sex chromosomes, and mitochondrial DNA.

A high-throughput system for tracking an animal and a meat product through a supply chain, the system includes the steps of: performing SNPTrack analysis of a plurality of samples obtained from a plurality of animals; obtaining and storing a plurality of SNPTracks in a database, wherein the database is searchable; obtaining a plurality of samples from meat products; performing SNPTrack analysis of the plurality of samples from meat products; and tracing the plurality of samples from meat products to the farm or the processing plant. The high-throughput system includes samples obtained from the plurality of animals prior to slaughtering in a farm or a processing center. The high-throughput system is capable of identifying and tracing animals and its products from birth to post-slaughter. The high-throughput system is capable of identifiying and tracing a meat product from a consumer to a farm of origin.

A software module includes instructions operative to provide a database comprising SNPtracks identified in TABLE 7.

A SNPTrack analysis kit to identify an animal includes: a plurality of oligonucleotides corresponding to a plurality of markers that contain one or more SNPs within a 0.2 to about 10 kb region of each marker; reagents to perform SNPTrack analysis; and access to compare SNPTracks with a plurality of SNPTracks in a database to identify the animal. The kit further includes an instruction manual to identify the animal.

A method of tracing an infected meat sample to a particular location, the method includes the steps of: performing SNPTrack analysis of a plurality of samples obtained from a plurality of animals with a plurality of markers, wherein the plurality of markers include one or more SNPs; obtaining and storing the SNPTracks of the plurality of samples in a database, wherein the database is searchable; performing SNPTrack analysis of the infected meat sample; comparing one or more SNPTracks of the infected meat sample with the SNPTracks of the plurality of the samples stored in the database; and tracing the infected meat sample to a particular location. The infected meat sample is a beef sample infected with mad cow disease or bovine spongiform encephalopathy (BSE). The infected meat sample is a sheep or goat sample infected with scrapie disease. The infected meat sample is an infected pork sample. The infected meat sample is obtained from a meat product in the market. The particular location can include farm of origin, production farm, processing center, retail outlet, distribution center, and whole saler.

A method of enhancing food safety and quality assurance of a meat product, the method includes the steps of: performing SNPTrack analysis of a plurality of samples obtained from a plurality of animals prior to slaughtering with a plurality of markers, wherein the plurality of markers include one or more SNPs; obtaining and storing a plurality of SNPTracks of the plurality of samples in a database, wherein the database is searchable; tracing the meat product to its source by performing SNPTrack analysis of the meat product and comparing a SNPTrack of the meat product with the SNPTracks of the plurality of samples stored in the database; and determining if the meat product is safe.

Definitions

Allele frequency: The frequency at which a particular allele (polymorphism) occurs in the members of a population under study.

Animal identification system: Information capable of being used to identify or trace a particular animal to pre-existing records. For example, an alpha-numeric code that is associated with a particular SNPTrack to identify a particular animal or a sample derived from that animal.

Database: An organized collection of information or data, stored preferably in an electronic computer readable format, that is capable of being updated and queried. The information stored in a database is managed by a database management system, which includes a software mechanism for managing that data. The database and its associated database management system can be accessed over the Internet or by any other electronic means. The database can store information or data including but not limiting to SNPTracks, farm of origin, production farm, processing plant, distribution center, retail outlet, wholesaler, breeding record, commercially valuable trait information, sibling information, offspring information, pedigree analysis, and other animal identification that in an organized and searchable manner.

Haplotype: A set of closely linked alleles (genes, genetic loci, or DNA polymorphisms) in a chromosome that is usually inherited as a single recombination unit. Some haplotypes may be in linkage disequilibrium. A haplotype may also be one of a set of single nucleotide polymorphisms along a region of a chromosome.

High-throughput system: A technique or a methodology or a platform capable of analyzing a plurality of samples simultaneously or in batches for a specific assay. For example, a plurality of DNA samples derived from blood samples of farm animals can be simulataneously analyzed for SNPs to obtain SNPTracks using a set of pre-defined assays.

Identity: A unique genetic identification of an individual by analyzing certain biological characteristics such as the DNA sequence, SNPTracks, and other genetic markers.

Identification: A method of determining a genetic identity of a mammal or a sample derived from a mammal, based on a comparison of SNPTracks with pre-existing SNPTracks of that mammal in a database (identity determination). Identification also includes traceability/tracing analysis that involves, for example determining the farm of origin or country of origin based on a comparison of SNPTracks obtained from a mammal or a sample derived from a mammal with pre-existing SNPTracks in a database obtained from matings pairs, maternal or paternal breeding populations. Identification of a mammal therefore involves determining the identity based on a unique SNPTrack and also tracing the mammal to a source or location based on SNPTracks of its maternal or paternal breeding populations.

Lineage: Genetic ancestry; Line of descent of the descendants from an original source of parentage.

Location: A place where a sample can be traced back for indentification purposes. A location can include a country, farm, production farm, processing plant, distribution center, retail outlet, wholesaler, or any other geographical territory.

Marker: A biomolecule that is capable of distinguishing biological samples. A marker can be a sequence of nucleotides.

Product: A portion of an animal, generally after slaughter, including processed meat sample that is available in a chain of commerce such as for example, a beef product at a grocery store. A processed meat sample includes any meat sample that is obtained post-slaughter.

Sample: Any material that can be analyzed to determine identity or traceability. Samples include processed meat samples, skin, blood, hair, bodily fluids or any other biological material obtained from a dead or live mammal. Sample also includes DNA or other genetic material that can be used for genotyping, haplotype determination, and SNPTrack analysis.

SNP: Single Nucleotide Polymorphism in a nucleotide sequence that includes insertions, deletions, and substitutions when compared between two or more members of a population. SNPs may be in the coding, non-coding, introns, exons, and the regulatory regions of DNA or RNA derived from mitochondrial, autosomal and sex-chromosomes.

SNPTrack: A SNPTrack includes a plurality of markers such that each marker includes a plurality of SNPs within a 0.2 to 10 kb interval in any region of a chromosome or mitochondrial DNA that may be inherited as a single unit during recombination if recombination occurs. The set of SNPs in a SNPTrack may also be in linkage disequilibrium, wherein the SNPs are linked.

System: An organized assembly or platform of components, resources, materials, tools, equipments, procedures or methods or processes or operations, software, interacting and funtioning in a unified way to perform a specific function.

Traceability/Tracing: A methodology to track a mammal to its farm of origin or country of origin or any other location by a genetic analysis. For example, a test meat sample or a test cattle may be traced to its farm or country of origin by excluding the sows that cannot be the mother of the test sample through an analysis of single nucleotide polymorphisms.

### BRIEF DESCRIPTION OF DRAWINGS

FIG. 1 is a database development scheme and the traceability of a test meat sample to its farm of origin using the database are shown (query). The schematic illustrations represent an identification scheme based on sow parentage.

FIG. 2 is a schematic representation of an identity determination model of a piglet. A database development scheme and the identity assay of a test meat product sample from a piglet using the database (query) are shown.

FIG. 3A shows a schematic representation of an identity/traceability determination scheme for a meat sample involving wholesalers and farms.

FIG. 3B shows a flowchart of of the various steps and instructions for identification of a meat product sample.

FIG. 4 is a representation of determining SNPTracks based on a two SNP example.

FIG. 5 demonstrates determining SNPTracks based on a three SNP example (FIG. 5A); for an offspring A (FIG. 5B); and for an offspring B (FIG. 5C).

### DETAILED DESCRIPTION

New methods and products designed to replace and/or complement existing methods to determine the identity (lineage) of an individual animal as well as traceability of the source, e.g. farm or country of origin are disclosed. Previous methods relied on the use of RFLPs, VNTRs and microsatellite markers. In contrast, present methods rely on the use of small DNA fragments that include single nucleotide polymorphisms (SNPs) in the mitochondrial genome, the autosomes, and the sex chromosomes. It relies further on the power of combining information from multiple SNPs which map within 0.2 to 10 kb of each other to identify the various SNPTracks present in the population and to determine their allele frequency. In an embodiment, pig genomic DNA was used to validate the method of using multiple SNPTracks and prove its utility in traceability and in determining identity. A number of swine breeds were used for this analysis including Duroc, Landrace, and Large White. However, the methods are suitable for all animals including mammals.

Because farms share semen for pig breeding, the farm of origin can only be identified based on DNA from the sows. Therefore, animals may only be traced to their farm of origin using the DNA inherited from their mothers. Identification of genetic markers (lineage specific, X-chromosome specific and autosomal,) with sufficient information content to allow the unambiguous identification of each animal is an aspect of the present invention.

Lineage markers are markers that are inherited from the mother or the father. For example, they are derived from the mitochondrial genome or the Y chromosome. The mitochondrial genome is maternally inherited, which means that all offspring inherit their mitochondria only from their mothers. There are a number of known polymorphisms in the mitochondrial genome including a region which exhibits a higher than expected level of variation in about 1 kb of DNA (D-loop region). Mitochondrial SNPs may not have the power to identify each animal, but they allow grouping the sows into several genetically related sets based on the polymorphisms of their mitochondrial DNA, and to tailor the development of additional markers to supplement a traceability methodology.

The Y chromosome is passed on from father to son. Male progeny inherit an exact copy of the Y chromosome from their father. Because it does not undergo recombination outside of the pseudoautosomal regions, it behaves as a locus similar in its inheritance to the mitochondrial genome. The Y has accumulated a number of sequence variations due to errors in DNA replication during evolution. Such variations can be scored as SNPs and help group the boars into several genetically related groups for further marker development.

SNPs represent single nucleotide variations at specific chromosomal locations. SNPs were determined by direct sequence analysis following amplification from genomic DNA. They range in frequency from very rare (<1%) to the very common (49%). The distribution and the allele frequency of SNPs may vary between breeds. Therefore, it is important to develop and validate SNP markers in a specific population under study.

Using SNPs in this type of analysis is to identify sequence variations that are relatively common in the study group. Even “common” SNPs usually have only two alleles limiting the genetic information content of each marker and requiring the genotyping of a large number of SNPs to provide for the level of confidence needed to identify an individual animal. For that reason, it is useful to combine information from multiple SNPs that reside physically in close proximity (within 0.2 to 10 kb in the DNA sequence). Such markers are unlikely to be separated by recombination during mating, and their genetic and physical location information are combined to generate a SNPTrack. The power of each SNP or a combination of SNPs to identify an individual in a population is determined statistically based on the data generated using a population under study.

Because the traceability of each animal will depend heavily on the sows, it is advantageous to emphasize X-chromosome markers in addition to the mitochondrial SNPs. Male progeny inherit their X chromosome from their mothers, while female progeny inherit one X chromosome from each parent. The mode of inheritance of X specific SNPs allows a more precise allele assignment increasing the power of each marker. Therefore, it is useful to develop a large number of X-chromosomes SNPs.

Additional markers are developed from autosomal genes to be used when needed to identify an individual or trace its farm of origin. The autosomal SNP markers are identified using the same strategy used for the X specific markers. For example, the mitochondria and the non-recombining portion of the Y represent two useful genetic areas. In an embodiment using pigs, sequencing of the mitochondria was not limited to the D-loop region, but SNP detection was performed in all mitochondrial genes. Additional markers were selected from known X chromosome genes including the amelogenin gene, and the androgen receptor gene. Autosomal markers were selected from mostly unlinked loci on various chromosomes. They included an IL-2 receptor, an obesity gene (leptin), and a fatty acid binding protein.

In an embodiment, additional DNA sequences for SNP detection were derived from pig DNA contained in bacterial artificial chromosomes (BACs) from the SRY locus on the Y chromosome and three X chromosome-specific genes. The BACs were subcloned to generate novel DNA sequences that were used to identify SNPs from different breeds of pigs.

Efforts focused on the identification of short DNA fragments (0.2 to 10 kb) which harbor multiple SNPs. The polymorphic information of multiple SNPs was combined to generate SNPTracks. The SNPs on individual chromosomes was determined using a number of methods. For example, the SNPTracks could be determined from homozygous individuals, subcloning and sequencing of individual haplotypes, pedigree analysis, and amplification using allele-specific nucleotides followed by sequencing. X chromosome markers can be determined by analyzing male animals because they are haploid for the non-recombining regions of the sex chromosomes.

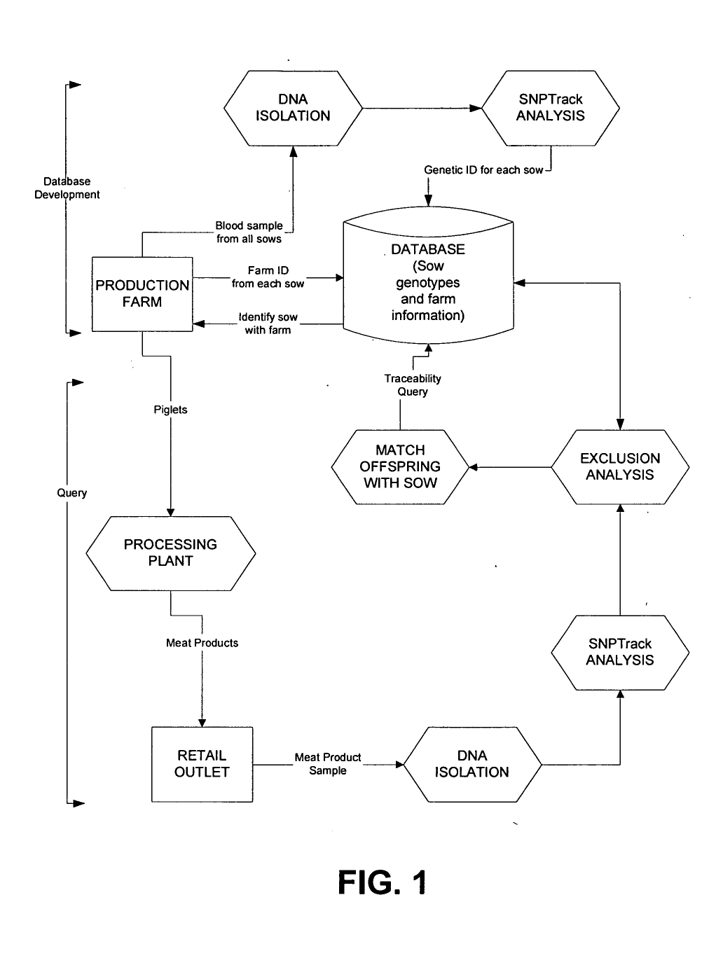

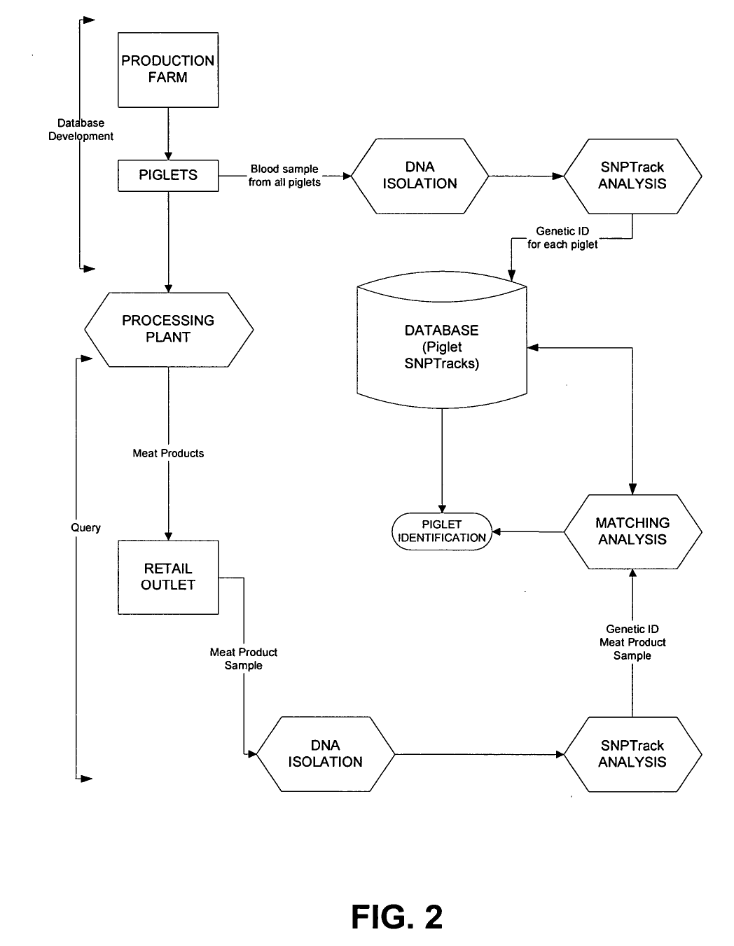

One of the differences between traceability and identity depends on which animals are genotyped and entered into a database. For example, traceability relates to the sows (mothers) and identity relates to the actual animals that are slaughtered (e.g., piglets or offsprings). In a traceability approach, a genetic identification of the meat sample is used to locate the mother by excluding who cannot be the mother (FIG. 1). In identity, a matching of genetic identification of the meat sample with those in the database is performed (provided SNPTrack analysis was performed for the animal before slaughter) to find that same genetic identification in the database.

Reduced heterozygosity in highly inbred populations limits a polymorphism's value. However, the methods that are used currently suffer from similar disadvantages. This limitation can be overcome by analyzing additional loci to increase the possibility of identifying polymorphic markers in the population under study. In farm animals, it is difficult to determine the haplotype frequencies with absolute certainty because of the number of breeds involved and the breeding practices of farm animals. This advantage can be overcome by selecting highly informative markers in most breeds and determining the haplotype frequencies in a relevant sample of the population to be analyzed.

A method of identifying an animal includes the steps of (1) obtaining a sample from the animal (for example blood sample from a cow prior to slaughter) or from a processed product (for example, a beef product in the market) of the animal; (2) performing single nucleotide polymorphism (SNP) analysis that includes a one or more markers, such that the markers include one or more SNPs-SNP analysis can be performed, for example, by isolating DNA from the sample, followed by PCR amplification using marker specific primers (e.g., listed in Table 3) and sequencing to determine the base pairs; (3) generating SNPTracks of the animal such that the SNPTracks contain one or more markers with one or more SNPs (SNPTracks combine the SNP data from multiple markers from (2)); and comparing the SNPTracks of the animal to a database that includes pre-existing SNPTracks to identify the animal (the database has been previously created using similar markers and performing SNPTrack analysis, for example, with samples obtained from animals in a farm). The SNPTracks can be generated by combining the individual SNP data from a plurality of markers in to one or more track that contains a string of SNPs from different regions of the genome (for example, a sample spectrum of genetic regions analyzed for developing swine markers is listed in Table 4).

The markers are designed and developed from autosomes, sex chromosomes, and mitochondrial DNA, wherein the SNPs in a marker are present within a nucleotide region of 0.2 to about 10 kb. Other genetic segments that span regions larger than 10 kb and shorter than 0.2 kb are also within the scope of this disclosure.

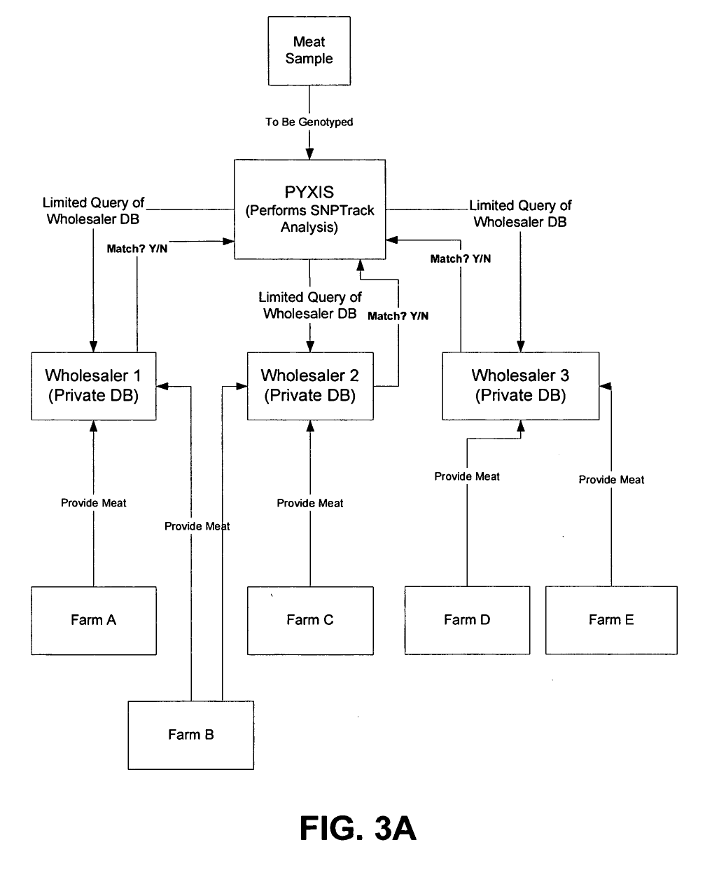

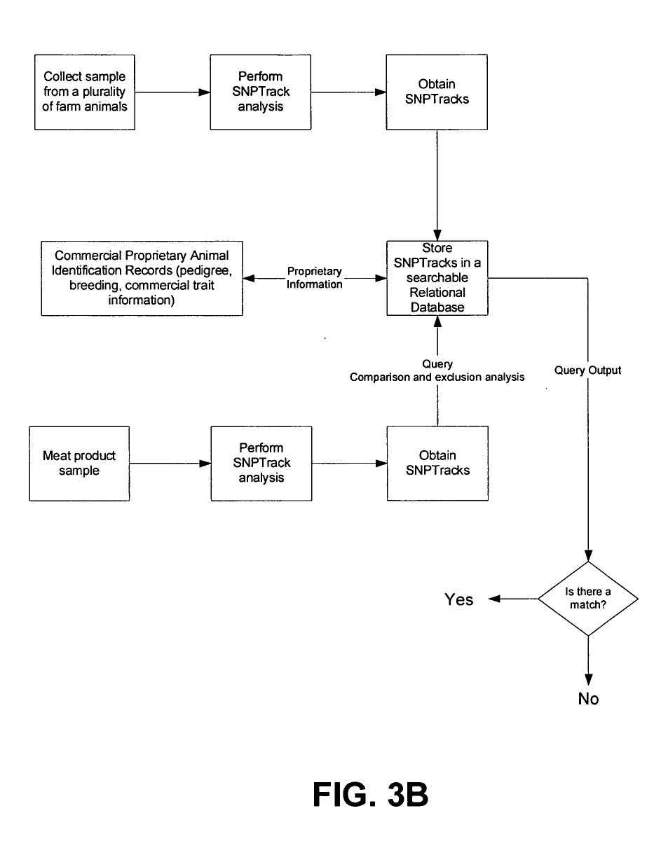

A method of genotyping or performing SNPTrack analysis to identify and/or trace a meat sample to a farm of origin is illustrated in FIG. 3A. In an illustration, a company, PYXIS performs a SNPTrack analysis to determine the genotype of a meat sample provided by a customer or obtained through any other source. The meat sample can include fresh or processed samples from pig, cows, and sheep. DNA is isolated from the sample using standard DNA isolation procedures and SNPTrack analysis is performed with a set of markers, such as, for example markers from Table 7.

The results of the SNPTrack analysis are queried against databases developed and maintained by wholesalers. For example, the genotype data obtained through SNPTrack analysis by PYXIS, are compared against pre-existing SNPTrack data in the databases developed by Wholesaler 1, 2 and 3. In the illustrated model in FIG. 3A, PYXIS has limited access to search and compare for matches in the Wholesaler databases. If the Wholesaler 1 database has a match (or no match) to a query by PYXIS, the Wholesaler 1 database will return an appropriate search result, e.g., “Match” or “No Match”.

The Wholesaler databases will have identification records to trace a particular meat sample to a farm of origin or to another upstream source such as another wholesaler. In the illustrated model in FIG. 3A, a Wholesaler can track a meat sample (if there is a “Match”) to a particular farm of origin. The wholesaler may inform the farm of origin and take appropriate measures to insure safety of the meat products that may contain an infected or defective meat sample that was tested.

In the model illustrated in FIG. 3A, the Wholesaler databases were developed by performing SNPTrack analysis of meat samples (sample collected from either slaughtered or prior to slaughtering) and the mating population from various farms. Thus, the databases may contain haplotype data (SNPTracks) of slaughtered meat samples, meat samples prior to slaughtering or culling, and mating population (breeding animals). PYXIS performs SNP analysis for the samples; develops the SNPTracks; and provides the software module to search and compare Wholesaler databases in a limited way. PYXIS also provides integrated product development solutions wherein a Wholesaler or a farm develops and independently maintains genetic identification databases based on SNPTracks for animals used in the food chain. Thus, PYXIS assists in the development of searchable databases of SNPTracks that are independently maintained by a larger user such as a Wholesaler and also provides an integration platform wherein a smaller user can have its sample analyzed and traced/identified to a farm of origin. PYXIS acts as an intermediate service provider to enable meat samples to be genotyped (or analyzed using SNPTracks) and identified or traced to a particular farm of origin. The larger user such as a Wholesaler independently maintains the database, thus insuring confidentiality of the breeding records and other valuable information.

In an embodiment, the PYXIS develops and maintains the Wholesaler databases in a single database management system. PYXIS maintains confidentiality among multiple Wholesaler databases and provides SNPTrack analysis to identify a meat sample. Thus, confidentiality is maintained among the multiple Wholesaler databases, expecially for commercially valuable and proprietary information.

The method shown in FIG. 3A may be performed in connection with a software module as generally depicted in FIG. 3B. The term “computer module” or “software module” referenced in this disclosure is meant to be broadly interpreted and cover various types of software code including but not limited to routines, functions, objects, libraries, classes, members, packages, procedures, methods, or lines of code together performing similar functionality to these types of coding. The components of the present disclosure are described herein in terms of functional block components, flow charts and various processing steps. As such, it should be appreciated that such functional blocks may be realized by any number of hardware and/or software components configured to perform the specified functions. For example, the present disclosure may employ various integrated circuit components, e.g., memory elements, processing elements, logic elements, look-up tables, and the like, which may carry out a variety of functions under the control of one or more microprocessors or other control devices. Similarly, the software elements of the present invention may be implemented with any programming or scripting language such as C, C++, SQL, Java, COBOL, assembler, PERL, or the like, with the various algorithms being implemented with any combination of data structures, objects, processes, routines or other programming elements. Further, it should be noted that the present disclosure may employ any number of conventional techniques for data transmission, signaling, data processing, network control, and the like as well as those yet to be conceived.

### EXAMPLES

### Example 1

### Determining Traceability and Identity and of Pigs

An objective of this example was to develop a genetic test to trace fresh and processed pork products back to the farm of origin, and to verify that the product was indeed from the farm stated to be the origin. A second objective was to trace the product back to the parent boar and sow, and thus from parentage records to the grandparents in the pure nucleus herd populations. The ability to determine parentage provides breeders with power to combine the information from genetic lineage and physical attributes to select animals with preferred traits for breeding programs and to eliminate animals responsible for poor quality.

a) Identification of SNPTracks

SNPTracks were determined by manually comparing the DNA sequence (0.2-10 kb) from the same genetic region (locus) across 60 different animals representing some of the major breeds used in pork production such as, for example, Duroc, Landrace, and Large White. A set of 100 to 200 SNP markers were identified based on the differences in the nucleotide sequence within the 0.2 to 10 kb region of DNA in either autosomal, sex chromosomes, or mitochondrial DNA. The sequences were available either in a proprietary database or were obtained by direct sequencing of desired regions. Differences (insertions/deletions/substitutions) among the DNA sequence were identified as SNPs. Approximately 100 genetic regions (markers) were evaluated for the presence of SNPs by comparing the sequences of each region from the 60 different animals. The selection of which genetic regions (markers) to be included in the test was accomplished by determining which markers were actually polymorphic (i.e. contained SNPs) in the target production population. Some of the markers that were polymorphic in the original 60 animals, were not polymorphic in the target population and thus were excluded. A set of 20 markers, each with a SNPTrack composed of 2 or more SNPs (a total of 60 SNPs), is based on the exclusion power predicted by theoretical calculations on the chance of miss identifying an unrelated animal based on chance (see TABLE 6).

B) Determining Allele Frequencies and Minimizing the Number of SNPTracks Needed for Identity or Traceability Studies

The most useful SNPs are those that are frequently represented in a population. A single SNP has two alleles. A SNP is most useful if the two alleles are present at equal frequency. Most SNPs have two alleles with frequencies between 20 and 40%. Combining multiple SNPs spanning 0.2 to 10 kb facilitates segregation as a single locus with 5 or 6 alleles (since it is unlikely that they will ever be separated by recombination). For a hypothetical sequence GGGAATATTTATTACCTAT(G/C)TTATATTGGA, allele 1 is GGGAATATTTATTACCTAT(G)TTATATTGGA (50%) and allele 2 is GGGAATATTTATTACCTAT(C)TTATATTGGA (50%). An ideal situation is where allele 1 and allele-2 are present at equal frequencies (i.e. 50%) A second SNP is identified (that occurred by random mutation). Since this arose after the first SNP, it is only present in one of the alleles.

The second SNP is denoted as GGGAATATTTATTACCTAT(C)TTATA(T/C)TGGA. Allele 1 remains the same GGGAATATTTATTACCTAT(G)TTATA(T)TGGA (50%). However, the original allele 2 is now either GGGAATATTTATTACCTAT(C)TTATA(T)TGGA (allele 2; 25%) or GGGAATATTTATTACCTAT(C)TTATA(C)TGGA (allele 3; 25%). Together (allele 2 and allele 3), the frequency is 50%. If the second SNP is present at equal frequencies, then the overall frequency of the 3 alleles is 50%, 25%, and 25%. A value of 50%, 30%, 20% reflects empirically determined data.

In the example above, haplotypes are assembled based on the combination of SNPs (SNPTrack) at each genetic region (locus/marker). The three SNPTracks above are GT, CT, and CC. If a different genetic region had the SNPTracks CT, AT, AG the nine possible haplotypes would be GT/CT, GT/AT, GT/AG, CT/CT, CT/AT, CT/AG, CC/CT, CC/AT, CC/AG, wherein the first SNPTrack represents one genetic region and the second SNP track represent a different genetic region. The SNPTracks and the approximate frequencies were determined by comparing the DNA sequences of the same genetic region from 60 different animals. The actual allele frequencies may be different for any given population and may change over time. The value of the markers in the prediction of parentage depends on their frequency, which then governs the total number of markers required for analysis.

An informative list of the characteristics of short amplified fragments (amplicons) with SNPs is shown in Table 3 and a description of genetic regions examined for SNPs is shown in Table 4. A marker is informative if there are multiple alleles present. In the example above, the informative marker was the one in which the 3 alleles were present in the boar and sow population. It could also be the case that in the target boar and sow population there was only a single allele represented. This is determined by directly determining the DNA sequence at the SNP positions in the DNA isolated from the target boar and sow animals. (i.e. empirically determined) SNPTracks are composed of 2 or more SNPs that are identified within a genetic region of approximately 0.2 to 10 kb. The allele frequencies of some of the short amplified fragments (amplicons) of Table 3 are shown in Table 4. Table 5 shows SNPTracks and allele frequency distribution for some of the amplicons generated by the primers listed in Table 3. A segregation analysis of the markers across the study population was done.

C) Developing a Database with SNPTrack Data for Sows and Sires from Various Farms

Using the set of markers identified Table 3, SNPTrack analysis was performed. The DNA obtained from each of the sow and sire was subject to SNP analysis using the oligonucleotide primers described in Table 3 and subsequent SNPTracks were identified. The data obtained from this analysis were used to develop a SNPTrack database that contained unique SNPTracks for each of the sows and sires that were genotyped with the set of markers identified in Table 5 (see FIG. 1).

d) Validation of the SNPTrack Data in a Sample Population

A validation assay for a sample of piglets was performed. For example, a sample of 2000 piglets representing-200 piglets per farm may be used for validation. A minimum of 10 commercial farms with 200 piglets per farm and a minimum of 30 dams per farm may be used. A minimum of 8 different sires per farm (same sire may be used on more than one farm) and on each farm, a minimum of 6 sows to be mated with an un-mixed semen from a single sire was used. The sire and dam of each litter in the study were recorded, along with the date of birth and farm. For mixed semen, the identities of all the contributing boars were listed. An ear tagging system was used to identify all study animals (piglets) with a unique number and an ear tissue sample for DNA extraction and for subsequent SNPTrack analysis was provided. All records included the unique identification number of the ear tag or any other suitable identification system. SNPTrack data from the piglets derived from the various farms were queried into the SNPTrack database with no prior knowledge of the farm of origin. The SNPTracks from the sample population were used to validate the SNPTrack database.

For a field study, however, not all animals need to be identified using the same set of markers. The exact marker set used is tailored for each animal tested to minimize the number of markers needed and to reduce the cost of testing 100 to 200 markers. A final outcome may be a set of markers that will be placed in groups of 5 to 8 on a branched tree. The markers used to identify or trace each animal may depend on the results of the first set of markers analyzed and so on. The grouping of the markers was done statistically based on the data generated to minimize the number of markers needed to trace each animal.

E) Testing a Meat Sample to Trace the Farm of Origin

The DNA from a meat sample to be tested was extracted and SNP analysis was performed for markers identified in TABLE 5. The resulting SNPTracks were queried in the SNPTrack database to trace the farm of origin. Based on the exclusion probabilities, the meat sample is traced to its farm of origin (see FIG. 1)

### Example 2

### Kits to Determine Identity or Farm of Origin

Kits to determine identity or farm of origin includes oligonucleotide primers for a set of SNP markers, suitable buffers, enzymes and any other biochemical components necessary to perform SNP analysis. A database enriched with SNP marker analysis of breeding animals from various farms is useful in determining the results obtained using the kits disclosed herein. For example, oligonucleotides, whose sequences are described in TABLE 3, is provided in a multi-well high-throughput format for SNP analysis along with suitable buffers and enzymes. PCR amplification followed by direct sequencing or any other form of SNP detection are implemented to develop SNPTracks for any given sample. The SNPTracks are then used to identify the sample or trace the sample's farm of origin.

### Example 3

### SNPTrack Application in Humans

The human SNP database contains over a million SNPs. Current validation has focused on sequence variation within genes. These could be within coding sequences or in the 5′ and 3′ untranslated region. SNPs within human genes also help identify SNPs in the pig homologs because they identify regions within genes that tolerate sequence variations.

Current SNP cataloguing in humans have focused on disease association. The methods disclosed herein are helpful in tracing humans to their country of origin for immigration- and for developing SNPTrack databases for security-related purposes. These methods are also helpful in reconstructing genealogical trees.

### Example 4

### Three-Tier Searching Approach for Pork Traceability Assay

Some of the DNA matching procedures include 1) mitochondrial matching test; 2) mating-sample DNA matching test using available mating information; 3) parent-sample DNA matching test, independent of mating information. These activities are independent procedures that are conducted simultaneously during the matching process.

The ‘mating-sample DNA matching test’ is a novel design to trace the sample to a specific location based on the mating-pair information. The ‘parent-sample DNA matching test’ is a paternity test.

The mitochondrial DNA matching test (MT test) involves a simple matching of mitochondrial genotypes to identify sows with the same mitochondrial genotype as the query sample.

The mating-sample DNA matching test (MS test) involves exhaustive DNA matching against each known mating pair. SNPTracks of various markers obtained for a particular sample are compared against a database populated with SNPTracks obtained from various mating pairs (breeding population). This test attempts to answer the question whether a particular sample came from an offspring of the mating pair. The sample is excluded if its DNA profile (SNPTrack) is incompatible with that of any mating pair. The mating-sample DNA matching test (MS test) possess higher exclusion power than paternity testing (E=0.28125, Q₁=3/16=0.1875, Q₀=1/8=0.125). The MS test requires about 63% of markers to achieve same exclusion power as paternity testing with a known sire and requires about 41% of markers to achieve same power as paternity testing without known sire. Implementation of the MS test requires a database of breeding records and marker genotypes (SNPTracks).

Derivation of exclusion probability under the MS test is shown in Table 1. Table 1 shows the derivation of exclusion probability (E) of the MS test assuming bi-allelic markers. Assuming equal allele frequency, E=0.28125. In comparison, the exclusion probability for paternity testing is Q₁=0.1875 if the sire is known and Q₀=0.125 if the sire is unknown. For the MS test, heterozygous offspring and parents all contribute to the exclusion power. For paternity test, heterozygous disputed parentage does not contribute to exclusion power.

TABLE 1
Exclusion probability (E) for mating-sample DNA matching test.

| | | | Excluded sample | | Exclusion |
|---|---|---|---|---|---|
| Dam | Sire | Mating freq | Genotype | Frequency | probability |
| AA | AA | p⁴ | Aa, aa | 1- p² | p⁴(1- p²) |
| AA | Aa | (2 pq)p² | aa | q² | (2 pq)p²q² |
| AA | aa | p²q² | AA, aa | 1-2 pq | p²q²(1-2 pq) |
| Aa | AA | (2 pq)p² | aa | q² | (2 pq)p²q² |
| Aa | Aa | (2 pq)² | — | 0 | 0 |
| Aa | aa | (2 pq)q² | AA | p² | (2 pq)p²q² |
| aa | AA | p²q² | AA, aa | 1-2 pq | p²q²(1-2 pq) |
| aa | Aa | (2 pq)q² | AA | p² | (2 pq)p²q² |
| aa | aa | q⁴ | AA, Aa | 1-q² | q⁴(1-q²) |

| | | |
|---|---|---|
| Sum | 1 | E |

$\begin{matrix}E = {p}^{4} ( 1 - {p}^{4} ) + {q}^{4} ( 1 - {q}^{2} ) + 4 {p}^{2} {q}^{2} ( 1 - pq ) \\ = 9 / 32 = 0.28125 if p = q = 1 / 2. 3 )\end{matrix}$

Possible genotypes for two bi-allelic markers based on direct matching of meat sample to all possible mating progeny (Mating test) is shown in Tables 2A and 2B.

Table 2C shows the genotypes of Sow 1 and Boar 1 for Markers 1 and 2.

TABLE 2A

| | |
|---|---|
| Marker 1 | Marker 2 |

| | | | | | | |
|---|---|---|---|---|---|---|
| Sow | | | | | | |
| 1 | C/C | G/G | C/A | G/C | T/A | A/A |
| Boar | | | | | | |
| 1 | C/A | G/A | C/A | C/C | A/A | G/A |

Potential alleles from mating between Sow 1 and Boar 1 (mitochondrial markers are excluded) is shown in Table 2C. One allele comes from Sow 1 and one allele comes from Boar 1.

TABLE 2C

| | |
|---|---|
| Marker 1 | Marker 2 |

| | | | | | |
|---|---|---|---|---|---|
| C/C | G/G | C/C | G/C | T/A | A/A |
| C/A | G/A | C/A | C/C | A/A | A/G |
| | | A/A | | | |

For Marker 1 there are 12 possible genotypes of the offspring

| | | | |
|---|---|---|---|
| 1) | C/C | G/G | C/C |
| 2) | C/C | G/G | C/A |
| 3) | C/C | G/G | A/A |
| 4) | C/C | G/A | C/C |
| 5) | C/C | G/A | C/A |
| 6) | C/C | G/A | A/A |
| 7) | C/A | G/G | C/C |
| 8) | C/A | G/G | C/A |
| 9) | C/A | G/G | A/A |
| 10) | C/A | G/A | C/C |
| 11) | C/A | G/A | C/A |
| 12) | C/A | G/A | A/A |

For Marker 2 there are 8 possible genotypes of the offspring

| | | | |
|---|---|---|---|
| 1) | G/C | T/A | A/A |
| 2) | G/C | T/A | A/G |
| 3) | G/C | A/A | A/A |
| 4) | G/C | A/A | A/G |
| 5) | C/C | T/A | A/A |
| 6) | C/C | T/A | A/G |
| 7) | C/C | A/A | A/A |
| 8) | C/C | A/A | A/G |

Considering only Markers 1 and 2, there are 96 possible (8×12) genotypes for their offspring. The meat sample genotype would be compared against these 96 possibilities to identify an exact match.

The parent-sample DNA matching test (PS test) involves exhaustive DNA matching against each potential parent in the absence of mating information. This test answers the question whether a disputed parent is the true parent of the known offspring. This test is implemented in the absence of any mating information. Therefore, knowing the sire significantly improves the power in identifying the dam.

These three tests may be performed sequentially or simultaneously. But care should be taken not to exclude the right mother because of a genotyping error.

### Example 5

### Determining SNPTracks-2 and 3 SNP Haplotypes

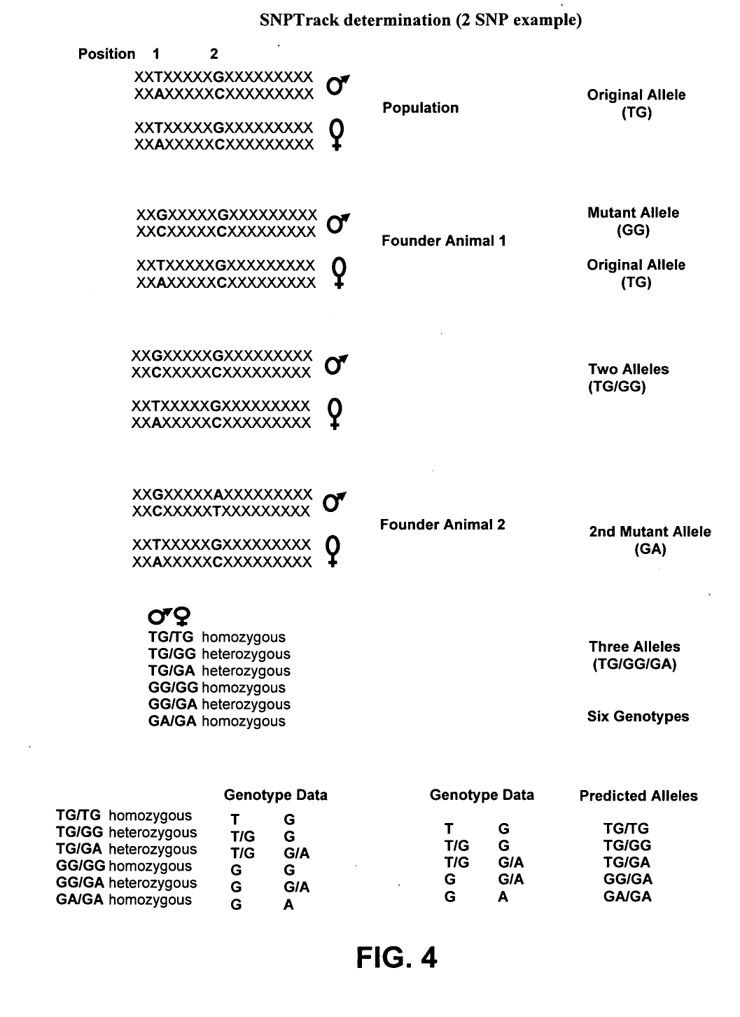

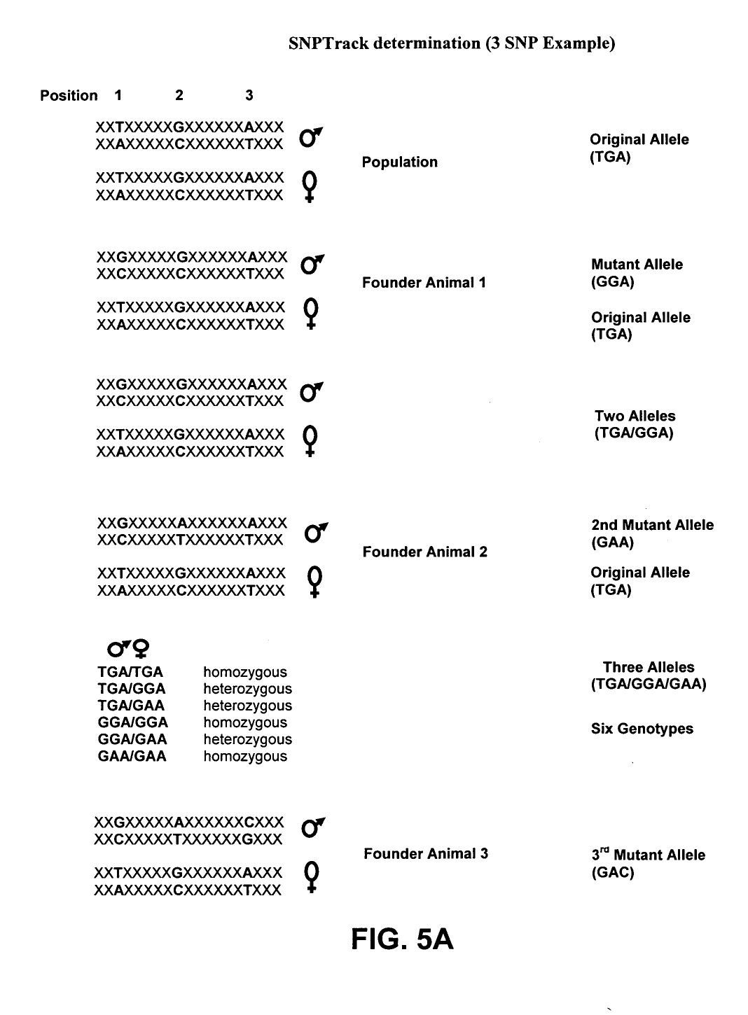

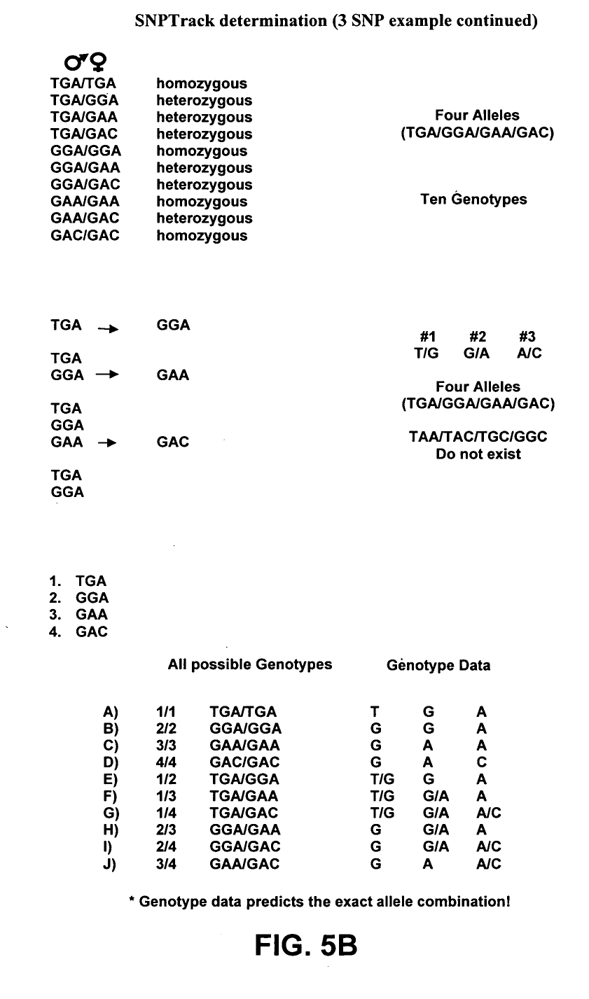

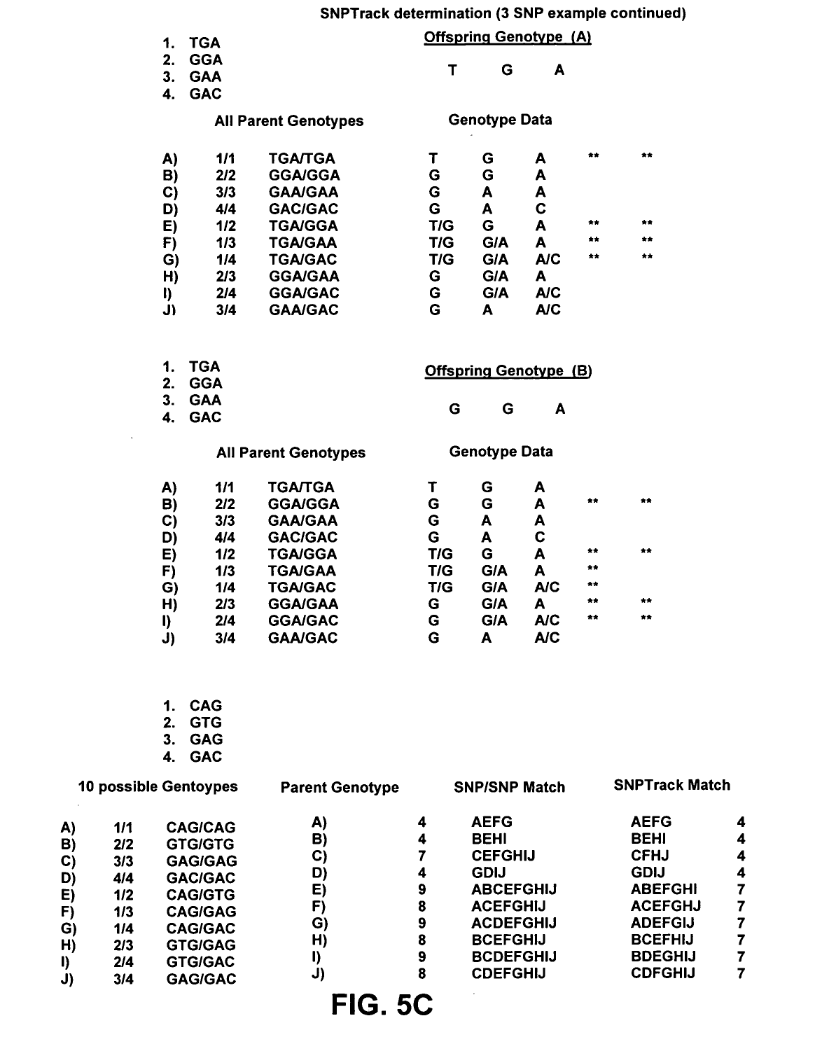

Determination of SNPTracks based on 2 or 3 SNP haplotype examples is illustrated in FIGS. 4, 5A-C. In FIG. 4, the population has 2 SNP allele at positions 1 and 2 of the SNPTrack designated “TG” respectively. The male and female symbols refer to the respective copy inherited from the father and the mother respectively. Each copy is shown as complementary double stranded DNA. For example, the original allele as indicated by reading the top strand is “TG” and is “AC” as indicated by reading the bottom strand. Therefore, depending upon which strand is read during the haplotype determination, the SNPTracks may vary because of base complementarity. The “X” denotes any intervening base between the SNPs. The “population” refers to a representative sample from the general population of a specific group of animals. The “founder animal” refers to an original animal that has a specific SNPTrack. As DNA is doubled-stranded, assays can be designed to detect the SNP on either strand. Therefore a SNP that is identified as a T/C, could also be detected as a A/G on the complementary DNA strand. Due to technical issues related to SNP detection technologies, an SNP assay may be designed to detect the complementary SNP rather than the indicated SNP.

In the illustrated example in FIG. 4, the “founder animal 1” has a mutant allele “GG” inherited from the father and the original allele “TG” inherited from the mother. The alleles denoted herein, unless specified otherwise, refer to the top strand in a double stranded DNA sequence. Founder animal 1 has two alleles—the original allele “TG” and the mutant allele “GG”. There can be another “founder animal”, such as, for example “founder animal 2” that has a second mutant allele “GA” inherited from the father and the original allele “TG” inherited from the mother. Therefore, the two founder animals have three alleles designated “TG/GG/GA”. These three alleles give rise to six possible genotypes as illustrated in FIG. 4. The genotype data for these three alleles are also illustrated in FIG. 4. For example, for the TG/GA heterozygous allele, the genotype data will be T/G and G/A at positions 1 and 2 of the SNPTrack respectively. The genotype data or the SNPTrack determination is unique for a specific allele pair. Thus, two founder animals with a total of three alleles for a SNPTrack that includes 2 SNPs, there are six possible genotypes that can be determined by a SNPTrack analysis. Offsprings generated between these founder animals, assuming the founder animals 1 and 2 are of opposite sex, will have one of the six possible genotypes. Because the possible genotypes of a sample can be predicted if the SNPTracks of the father and the mother are known, genotyping errors including sequencing errors can be corrected or filtered off from affecting the SNPTrack analysis. General assumptions in the haplotype or SNPTrack determination model discussed above include that the mutation events are independent; SNPs are close enough that recombination does not happen; the SNP at position 1 is always linked to the SNP at position 2. SNPs that are within 0.2 to 10 kb are assumed to segregate together and are considered linked.

A three SNP example is illustrated in FIGS. 5A-5C. In FIG. 5A, the three SNPs at positions 1, 2 and 3 are designated as “TGA”—the original allele in the population. The general descriptions for “founder animal”, “population” and other notations and nomenclature are the same as described for the 2 SNP example in FIG. 4. The founder animal 1 has an original allele TGA inherited from the mother and a mutant allele GGA inherited from the father. The founder animal 2 has 2^{nd} mutant allele GAA inherited from the father and the original allele TGA inherited from the mother. The three alleles from these founder animals 1 and 2 are designated “TGA/GGA/GAA”. The six possible genotypes derived from these three alleles are designated in FIG. 5A. These include three homozygous and three heterozygous genotypes.

In FIGS. 5A and 5B, founder animal 3 has a 3rd mutant allele GAC inherited from the father and an original allele TGA inherited from the mother. Therefore, among the founder animals 1, 2, and 3, there are four different alleles—one original allele and three mutant alleles. There are ten possible genotypes derived from these four alleles as illustrated in FIG. 5B. These include 4 homozygous and 6 heterozygous genotypes. In FIG. 5B, alleles TAA/TAC/TGC/GGC are shown not to exist as an illustration to demonstrate the predictive power of SNPTrack analysis, Because these alleles do not exist among the ten genotypes derived from the founder animals 1, 2, and 3, the offsprings from these founder animals also cannot have any of those alleles. The genotype data can therefore predict the exact allele combination to trace an offspring to a parent or to a particular location depending upon the database records. FIG. 5C is an illustration to demonstrate the power of SNPTrack analysis to identify parent genotypes based on the genotype data of sample offsprings (A) and (B). In FIG. 5C, the ten possible parent genotypes based on the four alleles (1. CAG; 2. GTG; 3. GAG; 4. GAC) are designated as A through J respectively. For example, for an offspring with the SNP designated by a genotype CAG, there are 4 possible matching parent genotypes (A, E, F, G) under both the SNP/SNP Match column (comparison done by a SNP match, that is if there is one matching SNP) and the SNPTrack Match column (comparison done by identifying a matching SNPTrack, that is all three SNPs must match). However, for an offspring with a genotype GAG, under the SNP Match column, there are 7 possible parent genotypes (C, E, F, G, H, I, J), whereas under the SNPTrack Match column, there are only 4 possible parent genotypes (C, F, H, J). Thus, SNPTrack analysis and exclusion is more powerful than convention SNP/SNP Match analysis. SNPTrack analysis in this example reduces the number of potential parents for further exclusion analysis.

Some of the general assumptions and observations in determining the SNPTrack analysis based on the three SNP example include independent mutations; SNPs are close enough that recombination does not occur; and new mutations occur only on a single previous allele.

FIGS. 4-5C illustrate SNPTrack determination and genotype analysis for two SNP and three SNP models. SNPTracks that have more than 3 SNPs and SNPTracks that include a plurality of 2 or 3 SNP haplotypes can also be designed and developed. For example, the dataset in Table 8 demonstrate the power of combining a plurality of 2 or 3 SNP haplotypes in developing SNPTracks based on approximately 15 markers from the pig genome.

Data shown in Table 8 illustrate a SNPTrack analysis performed with samples derived from a group of pigs that included mothers and their offsprings. The results of the SNPTrack analysis is shown in Table 8. Under the Animal I.D. column, for example, MLV1 represents the mother and the four following designations MLV1-P1 . . . P4 represent the 4 different offsprings. SNPTrack analysis was performed with a set of about 15 markers listed in Table 8. Positions indicated with “F” represent assay failures and were not included in the exclusion analysis. By comparing the SNPTracks of the offsprings against the SNPTracks of their mothers illustrate the power of SNPTrack analysis to identify the correct mother and eliminate the incorrect mothers. When the SNPTrack of a mother, such as, for example, MLV1 is known, an offspring such as MLV1-P1 can be identified and traced through its mother by comparing the SNPTrack obtained from MLV1-P1 with the SNPTracks stored in a database that also includes the SNPTrack obtained from MLV1, the mother. If the database includes the SNPTrack of MLV1-P1 itself (obtained and stored previously), then the offspring can be uniquely identified and traced to a particular location such as farm of origin.

A matching and a non-matching example wherein non-matching mothers are excluded is shown in Table 9. In the first example shown in Table 9, MLAC1 is an offspring and PGG1-5 are 5 possible mothers. SNPTrack analysis was performed and the SNPTracks were compared. In this assay, failures are indicated as N/A and some SNPs are indicated as D/I for deletion and insertion. None of the 5 mothers could be the parent of the sampe MLAC1 because they are excluded by the SNPTrack analysis. The excluded positions are highlighted in gray.

In the second example shown in Table 9, among the five possible mothers PGG1-5, only PGG4 could be the parent of the offspring MLAC2 because PGG1-3 and POG5 are excluded, with the excluded positions shown in grey highlights.

### Example 6

### Developing SNPTracks to Identify and/or Trace a Beef Sample to a Particular Location or a Farm of Origin

SNPTrack analysis disclosed herein can be adapted to traceability and identity assays to track beef products to a specific location or farm of origin. Based on the disclosure provided herein, SNPTracks that include a plurality of segments of SNPs in cow genome can be obtained from SNP sources such as the National Center for Biotechnology Information (http://www.ncbi.nlm.nih.gov/genome/guide/cow/).

Another source to obtain bovine SNPs is http://www.livestockgenomics.csiro.au/ibiss/ discussed in Hawken et al., (2004), An interactive bovine in silico database (IBISS). Mammalian Genome 15, 819-827.

TABLE 3
Characteristics of amplicons with SNPs from pig genome.

| | | | | |
|---|---|---|---|---|
| | Length of | | Amplicon | Annealing |
| Amplicon | the oligo | | size | temperature |
| Name | (bp) | Oligo Sequence | (bp) | (° C.) |

| | | | | |
|---|---|---|---|---|
| PBE3F | 23 | CTGACAGTTAAAGACTGCCCAAC | 658 | 63 |
| PBE3R | 24 | AGCTCCATGTCTTACTCATCCACC | | |
| ACY-F7 | 24 | GCAGCAATACTGTGCCTTAGAAAC | 976 | 65 |
| ACY-R7 | 24 | AAGTAATAGGGAGTGAGAGTCTCC | | |
| BG-F2 | 23 | ATCTCGACAACCTCAAGGGCACC | 1145 | 63 |
| BG-R2 | 24 | CCACTCACATGCGTGCTTTACAAC | | |
| COX2-F4 | 23 | TGGTGCCTGGTCTGATGATGTAC | 1148 | 63 |
| COX2-R4 | 24 | AGCAGAAAGCGCTTGCGGTATTCA | | |
| EG-F7 | 24 | CACCCTGCAGCATCTTCTTAGCTG | 1074 | 63 |
| EG-R7 | 24 | CCAAACTGAGGCCGGGTTGTGCTC | | |
| GALT-F4 | 22 | TGCAAGCATCCAGGGCTGCTTT | 1394 | 63 |
| GALT-R4 | 24 | CGAAACAAGTTCTAGTGAGCTCTG | | |
| IKBA-F5 | 23 | ACACGGAGTCAGAGTTCACAGAG | 1291 | 65 |
| IKBA-R5 | 24 | AGCAGAAGTAAGGTCCTGGCTGAA | | |
| LepR-F7 | 22 | AAACCGCTGCCTCCATCCAGTG | 1330 | 63 |
| LepR-R7 | 24 | AGGCTGGAGTACTCCAATTACTCC | | |
| LPL-F2 | 24 | TCCATGATAGGCTGCATCCTAGAA | 1244 | 63 |
| LPL-R2 | 24 | CCAGAGGTCACGGGAACAGAACTG | | |
| P450-F18 | 24 | TCCCTGTTCTCTATGGCCTGCTTC | 896 | 63 |
| P450-R18 | 24 | GATGTGGTGGTGCTGAACTCCAAG | | |
| PBE42F | 24 | TCTGAGCCTCATATTCTAATGGAC | 534 | 60 |
| PBE42R | 23 | CCTTTCACTGCAGAAGTTCCAGG | | |
| PBE43F | 25 | GATTGCAGTATTTTTTGTCTTGGAG | 704 | 63 |
| PBE43R | 23 | CTGAAAGATCCATCCATTTGTTC | | |
| PBE57F | 24 | ATGCTGCGATTTCTCTGGAGTTCC | 602 | 63 |
| PBE57R | 24 | TGTCCAGACGTTCCCTTTGGCTCC | | |
| PBE59F | 26 | TTGAAATCCCTACTTAAGTCCTGCTG | 717 | 63 |
| PBE59R | 27 | CAAGAGTAAATCATGCAAGGAAATGTG | | |
| PBE64F | 25 | TAGGACAGGAGAAGGATAACAAACC | 760 | 63 |
| PBE64R | 24 | AGTCCCTGGACATATGCAGGTTAG | | |
| PBE73F | 25 | AGATGATCATCGAGCTGTAGGATAG | 590 | 63 |
| PBE73R | 25 | TTCCAATCCTTTGTGAATATCTGGC | | |
| PBE77F | 24 | GCAGGGACAGCTCTGCCAGGGAAC | 529 | 63 |
| PBE77R | 27 | GTTACCTTACTTGAACCCTTTCTTTCC | | |
| PBE84F | 25 | AGAGAACCCTCAACTCTCAGCTGTG | 663 | 63 |
| PBE84R | 26 | TTCAGCCTTATTGAGGTATAGTTATC | | |
| PRKAG-F3 | 23 | CAAGAAGCAGAGCTTCGTGGGTG | 1043 | 63 |
| PRKAG-R3 | 24 | CGATGAGTCCATGAGCTTAGAACC | | |
| RYRA-F6 | 25 | GTGTAATCTGTTGGAGTATTTCTGT | 1339 | 63 |
| RYRA-R6 | 24 | TCGGTAAGATTATCATCTGACTTC | | |
| SCAMP1-F3 | 23 | TGAGCAGCAGGTCTGGAATCTAG | 1175 | 63 |
| SCAMP1-R3 | 24 | GAGTAGCCACAAGAATATACCAAG | | |
| VAN-F1 | 23 | CCACAATGCTTGCCTTTGCAGAG | 1272 | 63 |
| VAN-R1 | 24 | CGCATCACACAAATGTGTTCATGG | | |
| WSCR-F1 | 23 | GAGAGGCAGTGGGTCCAGACCAA | 1249 | 60 |
| WSCR-R1 | 24 | TCCAAGGTGGTGTGAGCTGACCTG | | |

TABLE 4
Description of Genetic Regions Examined for SNPs

| Name | Length (bp) | Description |
|---|---|---|
| AB049327S1 | 1470 | Sus scrofa IL7 gene for interleukin 7, exon 1, partial sequence. |
| SSIGFBPII2 | 1515 | Sus scrofa IGF binding protein-2 (IGF) gene, exons 2 and 3. |
| SSAJ3734 | 1551 | Sus scrofa SCAMP1 gene, exon 1 and joined CDS. |
| AF252874 | 1638 | Sus scrofa bactericidal permeability increasing protein (BPI) gene, partial cds. |
| PIGMEF2A2 | 1768 | Sus scrofa domestica myocyte enhancer factor 2A (MEF2A) gene, exon and partial cds. |
| AF329087 | 1835 | Sus scrofa Niemann-Pick type C1 protein gene, promoter region and partial cds. |
| AF415201S2 | 1839 | Sus scrofa alpha-1,3-galactosyltransferase gene, exons 2 and 3. |
| SSIGFBPII1 | 1861 | Sus scrofa IGF binding protein-2 (IGF) gene, exon 1. |
| SSC309827 | 1933 | Sus scrofa partial ABCD3 gene for peroxisomal membrane protein 1, exons 13-14. |
| SSC430415 | 2053 | Sus scrofa partial GLUL gene for glutamate-ammonia ligase, exons 3-4. |
| AF019044 | 2132 | Sus scrofa DAX-1 gene, partial cds. |
| SSBETAG | 2392 | Sus scrofa beta-globin gene. |
| SSC344137 | 2395 | Sus scrofa partial ATP1A1 gene for Na+/K+ ATPase alpha 1 subunit, exons 13-16. |
| AF331845 | 2443 | Sus scrofa androgen receptor gene, promoter region and 5′ untranslated region, partial |
| | | sequence. |
| SSC2BTXNS | 2472 | S. scrofa C2 gene (exons 13-18) and BF gene (exons 1-2). |
| AF415201S1 | 2695 | Sus scrofa alpha-1,3-galactosyltransferase gene, exon 1. |
| AF202775 | 2798 | Sus scrofa androgen receptor mRNA, complete cds. |
| SSC404884 | 3032 | Sus scrofa partial cdkn3 gene, exons 7-8. |
| AF247680 | 3101 | Sus scrofa immunoreceptor DAP12 gene, complete cds. |
| SSAJ3742 | 3265 | Sus scrofa SCAMP1 gene, exon 9. |
| SSDNAMYO | 3506 | S. scrofa myogenin gene. |
| AY028583 | 3621 | Sus scrofa prostaglandin G/H synthase-2 (PGHS-2) mRNA, complete cds. |
| SSMYF5G | 3680 | Sus scrofa myf-5 gene and 3 microsatellite sequences. |
| AY044189 | 3733 | Sus scrofa uroplakin II gene, complete cds. |
| AF430245 | 3823 | Sus scrofa vanin-1 gene, promotor region, exon 1, intron 1 and partial cds. |
| SSC249746EPO | 3874 | Sus scrofa epo gene for erythropoietin, exons 1-5. |
| AF492499 | 4161 | Sus scrofa obese (ob) gene, intron 1. |
| SSU96150 | 4631 | Sus scrofa tear lipocalin/von Ebner's lingual gland protein (LCN1) gene, complete cds. |
| SSC6076 | 4911 | Sus scrofa HSL gene, exons 6 to 9 and 3′ UTR. |
| AY237828 | 5121 | Sus scrofa vanin-1 gene, promoter region, 5′UTR, and partial cds. |
| E15380 | 5418 | Porcine MCP promoter. |
| SSIKBAGE | 5764 | S. scrofa IkBa gene. |
| AF214521 | 5888 | Sus scrofa AMPK gamma subunit (PRKAG3) gene, complete cds. |
| AF328419 | 6239 | Sus scrofa amelogenin gene, exons 3, 4a, 4b, 5, 6, and 7a. |
| AF458070 | 6337 | Sus scrofa bone morphogenetic protein 15 (BMP15) gene, exons 1 and 2 and partial cds. |
| SSU14331 | 6511 | Sus scrofa myogenin gene, complete cds. |
| AY116585 | 6727 | Sus scrofa inhibin beta B precursor subunit (INHBB) gene, exons 1 and 2, complete cds. |
| SSTNFAB | 7218 | Porcine TNF-alpha and TNF-beta genes for tumour necrosis factors alpha and beta, |
| | | respectively. |
| AC090553trunk | 8043 | |
| AY112657 | 8053 | Sus scrofa fibrinogen-like protein 2 (FGL2) gene, complete cds. |
| SSY16039 | 8144 | Sus scrofa A-FABP gene for fatty acid-binding protein, exons 1-5. |
| AC091506trunk | 8212 | |
| AF036005 | 8480 | Sus scrofa interleukin-2 receptor alpha chain gene, partial cds. |
| AF535216 | 8660 | Sus scrofa endothelial nitric oxide synthase (NOS) gene, exons 3 through 14 and partial |
| | | cds. |
| AB017196 | 9361 | Sus scrofa ACY-1 and rpL29/HIP genes, complete cds. |
| SSC404883 | 9923 | Sus scrofa partial cdkn3 gene, exons 1-6 and join CDS. |
| SSC296176 | 10281 | Sus scrofa LIF gene for leukemia inhibitory factor. |
| SSC315771 | 12715 | Sus scrofa CTSL gene for cathepsin L, exons 1-8. |
| SSC7302 | 15604 | Sus scrofa triadin gene. |
| AL773560P | 17040 | Pig DNA sequence from clone containing cytochrome P450-21-hydroxylase |
| SSPPK | 19298 | S. scrofa ppk98 gene. |
| SSRYRA | 17808 | S. scrofa gene for skeletal muscle ryanodine receptor. |

TABLE 5
Allele frequency distribution

| | No. | | | | | | | | |
|---|---|---|---|---|---|---|---|---|---|
| | of | SNPTrac | | SNPTrac | | SNPTrac | | SNPTrac | |
| Marker | SNPs | k 1 | Frequency | k 2 | Frequency | k 3 | Frequency | k 4 | Frequency |
| EG | 3 | CGC | 46% | CAC | 22% | TAC | 17% | CAT | 15% |
| RYRA | 3 | ACA | 36% | ACC | 22% | AGC | 22% | CCC | 22% |
| VAN | 3 | CGC | 60% | CAC | 19% | TGT | 13% | CGT | 8% |
| WSCR | 3 | GCG | 44% | AAA | 22% | GAG | 18% | GAA | 16% |
| BG | 3 | CCC | 35% | CAC | 30% | TCC | 24% | CCT | 11% |
| LEPR | 2 | AG | 25% | AC | 25% | CC | 25% | GC | 25% |
| IKBA | 3 | CAG | 38% | CTG | 26% | TAG | 26% | CAT | 10% |
| COX2 | 3 | TAT | 54% | TGT | 17% | CAC | 17% | CAT | 12% |
| GALT | 3 | ACC | 47% | GGG | 24% | GGC | 17% | GCC | 17% |
| PRKAG | 3 | GAG | 43% | GGA | 27% | AAG | 20% | GAA | 11% |
| SCAMP | 3 | TAT | 33% | TGA | 26% | TGT | 22% | CGT | 19% |
| P450 | 3 | CGG | 40% | CAG | 23% | TGG | 21% | CGA | 16% |
| PBE42 | 3 | AGC | 48% | AGT | 27% | GGT | 17% | ATC | 8% |
| PBE43 | 3 | −CT | 41% | +CC | 21% | −AT | 25% | −CC | 14% |
| PBE3 | 3 | G−G | 34% | A+G | 33% | G−(gg) | 18% | G+G | 15% |
| PBE64 | 3 | GGC | 39% | GCT | 34% | GGT | 27% | AGT | 6% |
| PBE84 | 3 | GTC | 40% | GCC | 28% | TCC | 22% | GTT | 10% |
| PBE57 | 3 | CAT | 50% | TAC | 22% | CGC | 15.50% | CAC | 12.50% |
| PBE59 | 2 | TC | 50% | CC | 30% | CT | 20% | na | na |
| ACY | 2 | GC | 52% | GT | 30% | CC | 18% | na | na |

TABLE 6
SNPTrack Exclusion Probabilities

| Marker type | Exclusion probability | n₂ | n₁ |
|---|---|---|---|
| Autosomal markers | Q = 1 − 10⁻⁶ | 12.4 | 16.8 |
| only | Q = 1 − 10⁻⁷ | 14.5 | 19.5 |
| | Q = 1 − 10⁻⁸ | 16.6 | 22.3 |
| | Q = 1 − 10⁻⁹ | 18.6 | 25.1 |
| Autosomal markers | Q = 1 − 10⁻⁶ | 10.4 | 14 |
| and mitochondrial | Q = 1 − 10⁻⁷ | 12.4 | 16.8 |
| DNA typing | Q = 1 − 10⁻⁸ | 14.5 | 19.5 |
| | Q = 1 − 10⁻⁹ | 16.6 | 22.3 |

Number of autosomal markers required to achieve a given exclusion probability (Q):
n₂ = number of autosomal markers required when both the alleged dam and sire have marker genotypes;
n₁ = number of autosomal markers required with the alleged dam has marker genotypes by the alleged sire does not have marker genotypes.

TABLE 7
Marker Assignment/Position

| | No. | | SNP | | | | | | | |
|---|---|---|---|---|---|---|---|---|---|---|
| Marker | SNPs | Old Marker | positions | | | | | | | |
| P1SMIT | 8 | Mitochondrial | 15542 C/T | 15558 | 15615 | 15616 | 15675 | 15714 | 15840 | 16127 |
| | | | | A/T | C/T | C/T | C/T | C/T | C/T | G/A |
| P1S001 | 2 | ACY-STS7 | 245 G/C | 421 C/T | | | | | | |
| P1S002 | 3 | Cox2 | 368 C/T | 533 G/A | 939 C/T | | | | | |
| P1S003 | 3 | EG-STS7 | 774 G/A | 805 G/A | 817 G/A | | | | | |
| P1S004 | 3 | GALT | 478 G/A | 758 C/G | 866 C/G | | | | | |
| P1S005 | 3 | IKBA | 4476 C/T | 4679 T/A | 4904 G/T | | | | | |
| P1S006 | 2 | LepR | 426 A/C/G | 810 G/C | | | | | | |
| P1S007 | 3 | P450-STS18 | 71 C/T | 138 G/A | 361 G/A | | | | | |
| P1S008 | 3 | PBE3 | 115 G/A | 192 A/T | 555 T/G | | | | | |
| P1S009 | 3 | PBE42 | 111 G/A | 118 T/G | 181 C/T | | | | | |
| P1S010 | 3 | PBE43 | 314 C/T | 471 C/A | 524 C/T | | | | | |
| P1S011 | 4 | PBE 57 | 75 C/T | 109 G/A | 197 C/T | 268 | | | | |
| | | | | | | T/G | | | | |
| P1S012 | 2 | PBE59 | 276 C/T | 494 C/T | | | | | | |
| P1S013 | 3 | PBE 64 | 115 G/A | 419 C/G | 515 C/T | | | | | |
| P1S014 | 4 | PBE 73 | 93 A/C | 116 A/G | 177 C/T | 477 | | | | |
| | | | | | | C/T | | | | |
| P1S015 | 3 | PBE 84 | 14 A/G | 97 T/C | 428 T/C | | | | | |
| P1S016 | 4 | PBE132 | 102 C/G | 127 A/G | 193 C/T | 371 | | | | |
| | | | | | | A/G | | | | |
| P1S017 | 3 | PBE137 | 121 C/T | 278 C/T | 409 A/G | | | | | |
| P1S018 | 3 | PRKAG-STS3 | 1845 G/A | 1938 G/A | 2050 G/A | | | | | |
| P1S019 | 3 | RYRA-STS6 | 402 A/C | 408 C/G | 567 A/C | | | | | |
| P1S020 | 5 | SCAMP | 184 C/T | 389 G/A | 516 C/T | 582 | | 939 | | |
| | | | | | | G/A | | A/T | | |
| P1S021 | 4 | VAN-STS1 | 889 C/T | 950 C/T | 1009 G/A | 1065 | | | | |
| | | | | | | C/T | | | | |
| P1S022 | 2 | WSCR-STS1 | 411 C/T | 599 C/T | | | | | | |
| P1S023 | 4 | BG | 1257 C/T | 1323 C/T | 1425 C/A | | | 1966 | | |
| | | | | | | | | C/T | | |
| P1S024 | 3 | AMG | 907 A/C | 975 A/G | 1467 A/G | | | | | |
| P1S025 | 3 | LCN | 87 C/T | 373 G/C | 402 C/T | | | | | |
| P1S026 | 2 | CTSL | 252 A/G | 272 A/G | | | | | | |
| P1S027 | 2 | PBE112 | 61 C/T | 87 C/T | | | | | | |
| P1S028 | 3 | MYF5 | 1833 A/G | 2204 | 2335 A/C | | | | | |
| | | | | C/G | | | | | | |

TABLE 8
SNPTrack Analysis of Mothers and Offsprings

| . | PBE3 | | | ACY-STS7 | | EG-STS7 | | | RYRA-STS6 | | | PBE59 | | PBE43 | | | GALT | | | VAN-STS1 | | | IKBA | | |
|---|---|---|---|---|---|---|---|---|---|---|---|---|---|---|---|---|---|---|---|---|---|---|---|---|---|
| | G/A | A/T | T/G | G/C | C/T | G/A | G/A | G/A | A/C | C/G | A/C | C/T | C/T | C/T | C/A | C/T | G/A | C/G | C/G | C/T | G/A | C/T | C/T | T/A | G/T |
| Animal I.D. | 115 | i193IN | 555 | 150 | 326 | 773 | 804 | 817 | 402 | 408 | 567 | 276 | 494 | 4INDI | 471 | 524 | 478 | 758 | 866 | 950 | 1009 | 1065 | 4476 | 4679 | 4904 |
| MLV1 | G | NT | T | G | C/T | G | G/A | G/A | A | G | C | C | T | C | C/A | C/T | G/A | C/G | C/G | C/T | G | C/T | C/T | T/A | G |
| MLV1-P1 | G/A | A/T | T | G | C/T | G | A | G/A | A/C | C/G | A/C | C | C/T | C/T | C/A | C/T | G/A | C/G | C | C | G/A | C | C/T | A | G |
| MLV1-P2 | G/A | T | T | G | C/T | G | A | G/A | A/C | C/G | A/C | C/T | C/T | C | C/A | C/T | G/A | C/G | C/G | F | F | F | C/T | A | G |
| MLV1-P3 | G/A | T | T | G | C/T | G | A | G/A | A/C | C/G | C | C/T | T | C | C/A | C/T | G | C/G | C/G | F | F | F | C/T | A | G |
| MLV1-P4 | G/A | A/T | T | G | C/T | G | G/A | G | A/C | C/G | C | C | T | C | C/A | T | G/A | C/G | C/G | C | G/A | C | C/T | A | G |
| MLV2 | G/A | A/T | T | G/C | C/T | G | G | G | A | C | A/C | C | T | C | C/A | T | G | C/G | C/G | C | G/A | C | C/T | T/A | G |
| MLV2-P1 | A | T | T | G/C | C/T | G | G/A | G | A/C | C | A/C | C | C/T | C | C/A | T | G | G | C/G | C | G/A | C | C | T/A | G |
| MLV2-P2 | A | T | T | C | C | G | G/A | G | A | C | A/C | C/T | T | C/T | C | C/T | G | C | C | C | G/A | C | C/T | A | G |
| MLV2-P3 | G/A | A/T | T | G/C | C/T | G | G/A | G | A/C | C | A/C | C | C/T | C | A | T | G | G | C/G | C | G | C | C/T | A | G |
| MLV2-P4 | G | A | T | G | T | G | G/A | G | A/C | C | A/C | C | C/T | C/T | C/A | C/T | G | C/G | C | C | G/A | C | C/T | A | G |
| MLV3 | G/A | T | T | G | T | G | G | G | A | C | A | C | C/T | C | C | C/T | G/A | C/G | C/G | C | G | C | C | A | G/T |
| MLV3-P1 | A | T | T | G | T | G | G/A | G | A/C | C | A/C | C | C/T | C | C/A | T | G/A | C/G | C | C | G | C | C | A | G/T |
| MLV3-P2 | A | T | T | G/C | C/T | G | G/A | G | A/C | C | A/C | C | T | C | C | T | G/A | C/G | C/G | C/T | G | C/T | C | T/A | G/T |
| MLV3-P3 | G | A/T | T | G | C/T | G | G/A | G | A/C | C | A/C | C | C/T | C/T | C | C | G/A | C/G | C | C | G/A | C | C | A | G/T |
| MLV3-P4 | A | T | T | G/C | C/T | G | G/A | G | A/C | C | A/C | C | T | C | C | T | G/A | C/G | C/G | C | G/A | C | C | A | G |
| MLV4 | G/A | T | T | C | C | G | A | G/A | A/C | C | A/C | C | C | C | A | T | A | C | C | C | G/A | C | C | A | G |
| MLV4-P1 | A | T | T | G | C | G | A | G/A | C | C | C | C | C | C | C | T | A | C | C | C/T | G | C/T | C | A | G |
| MLV4-P2 | A | T | T | C | C | G | G/A | G | A | C | A/C | C | C | C | C | T | A | C | C | C/T | G | C/T | C | A | G |
| MLV4-P3 | A | T | T | G | C | G/A | G/A | G | A | C | A/C | C | C | C | C/A | T | G/A | C/G | C/G | C/T | G | C/T | C | T/A | G |
| MLV4-P4 | G/A | T | T | G/C | C | G | G/A | G/A | A/C | C | C | C | C/T | C | C/A | T | A | C | C | C/T | G/A | C/T | C | T/A | G |
| MLV5 | G | A/T | T | G/C | C | G | G/A | G/A | A | C | A/C | C | C/T | C/T | C/A | C/T | G/A | C/G | C | C | G | C | C | T/A | G |
| MLV5-P1 | G | A | T | G | C | G | G/A | G | A/C | C | A/C | C | C/T | C/T | C/A | C/T | G/A | C/G | C | C | G/A | C | C | A | G |
| MLV5-P2 | G/A | A/T | T | G/C | C/T | G | A | G/A | A/C | C | A/C | C | C | C | A | T | G/A | C/G | C | C | G | C | C | A | G |
| MLV5-P3 | G | A | T | G/C | C | G | G/A | G | A | C | A | C | C/T | C/T | C/A | C/T | G | G | C | C | G | C | C | A | G |
| MLV5-P4 | G/A | A/T | T | C | C | G | A | G/A | A/C | C | A/C | C | T | C | C/A | T | G/A | C/G | C/G | C | G/A | C | C | T/A | G |
| MLV6 | G/A | T | T | G | C/T | G | A | G/A | A/C | C | C | C | C/T | C | C | T | G/A | C/G | C/G | C | G/A | C | C/T | T/A | G |
| MLV6-P1 | G/A | A/T | T | G | C | G | A | G/A | A/C | C | C | C | C | C | C | T | G/A | C/G | C/G | C | A | C | C | T | G |
| MLV6-P2 | A | T | T | G | C/T | G | A | G/A | A/C | C | C | C | C | C | C | T | G | G | G | C | G/A | C | C/T | T/A | G |
| MLV6-P3 | G | A/T | T | G | C/T | G | A | G/A | A/C | C | C | C | C | C | C | T | G/A | C/G | C/G | C | G | C | C | T | G |
| MLV6-P4 | G/A | A/T | T | G | C/T | G | A | G/A | A | C | C | C | C/T | C | C | T | G/A | C/G | C/G | C | G/A | C | C/T | A | G |
| MLV7 | G/A | A/T | T | G/C | C/T | G | G/A | G/A | A | C/G | A/C | C | T | C/T | C/A | C/T | G/A | C/G | C/G | C | G | C | C | T/A | G/T |

| | PBE64 | | SCAMP | | | | LEPR | | COX2 | | | P450 | | | MITOCHONDRIAL | | | | | | | |
|---|---|---|---|---|---|---|---|---|---|---|---|---|---|---|---|---|---|---|---|---|---|---|
| | C/G | C/T | C/T | G/A | C/T | G/A | C/G | G/A/C | C/T | G/A | C/T | C/T | G/A | G/A | C/T | A/T | C/T | C/T | C/T | C/T | C/T | G/A |
| Animal I.D. | 419 | 515 | 184 | 389 | 516 | 582 | 426 | 810 | 368 | 533 | 939 | 71 | 138 | 361 | 15543 | 15558 | 15615 | 15616 | 15675 | 15714 | 15840 | 16127 |
| MLV1 | C/G | C/T | C | G/A | T | G/A | C/G | G/A | T | G/A | T | C | A | G | T | T | C | T | T | C | T | G |
| MLV1-P1 | C/G | C/T | C/T | G/A | T | G | C/G | G/A | T | G/A | T | C | G/A | G | T | T | C | T | T | C | T | G |
| MLV1-P2 | C/G | C/T | C/T | G/A | T | G | C/G | G/A | T | G/A | T | C | G/A | G | T | T | C | T | T | C | T | G |
| MLV1-P3 | C/G | T | C/T | G/A | T | G | C/G | A | T | G/A | T | C | G/A | G | T | T | C | T | T | C | T | G |
| MLV1-P4 | C | T | C | G | T | G | C/G | G/A | T | A | T | C | G/A | G | T | T | C | T | T | C | T | G |
| MLV2 | G | C | C/T | A | T | G | C/G | A | T | G/A | T | C | A | G | T | T | C | T | T | C | T | G |
| MLV2-P1 | G | C | T | A | T | G | C/G | A | T | A | T | C | G/A | G | T | T | C | T | T | C | T | G |
| MLV2-P2 | C/G | C/T | C/T | A | T | G/A | C/G | A | T | G/A | T | C | G/A | G/A | T | T | C | T | T | C | T | G |
| MLV2-P3 | G | C/T | C | A | T | G | G | A | T | A | T | C | G/A | G | T | T | C | T | T | C | T | G |
| MLV2-P4 | G | C | T | A | T | G | G | A | T | G | T | C | G/A | G/A | T | T | C | T | T | C | T | G |
| MLV3 | G | C | C/T | A | T | G/A | C/G | G/A | C/T | A | T | C | G/A | G/A | C | A | C | T | C | C | C | G |
| MLV3-P1 | G | C/T | C/T | A | T | G | C/G | G/A | T | G/A | T | C | G | G/A | C | A | C | T | C | C | C | G |
| MLV3-P2 | G | C/T | C | A | T | A | C | G/A | T | A | T | C | G/A | G | C | A | C | T | C | C | C | G |
| MLV3-P3 | G | C | C/T | A | T | G/A | C/G | G/A | C/T | A | T | C | G/A | G | C | A | C | T | C | C | C | G |
| MLV3-P4 | G | C | C/T | A | T | G/A | C/G | A | C/T | G/A | T | C | G | G/A | C | A | C | T | C | C | C | G |
| MLV4 | G | T | C/T | A | T | G | C | A | T | A | T | C | A | G | T | T | C | T | T | C | T | G |
| MLV4-P1 | G | C/T | T | A | T | G | C/G | A | T | A | T | C | G/A | G/A | T | T | C | T | T | C | T | G |
| MLV4-P2 | G | C/T | T | A | T | G | G | A | T | A | T | C | A | G | T | T | C | T | T | C | T | G |
| MLV4-P3 | G | T | C/T | A | T | G | G | A | T | A | T | C | A | G | T | T | C | T | T | C | T | G |
| MLV4-P4 | G | C/T | C/T | A | T | G | C/G | A | T | A | T | C/T | G/A | G | T | T | C | T | T | C | T | G |
| MLV5 | C/G | C/T | C/T | A | T | G/A | C/G | A | T | A | T | C | G/A | G | T | T | C | T | T | C | T | G |
| MLV5-P1 | C/G | C/T | C/T | A | T | G/A | C/G | A | T | G/A | T | C | G/A | G | T | T | C | T | T | C | T | G |
| MLV5-P2 | C/G | T | C | A | T | G/A | G | A | T | A | T | C | G/A | G | T | T | C | T | T | C | T | G |
| MLV5-P3 | G | C/T | T | A | T | G | C/G | A | T | A | T | C | G/A | G | T | T | C | T | T | C | T | G |
| MLV5-P4 | C/G | C/T | C/T | G/A | T | G | C/G | A | T | A | T | C | A | G | T | T | C | T | T | C | T | G |
| MLV6 | C/G | C/T | C/T | G/A | T | G | C | G/A | C/T | A | C/T | C/T | G/A | G | C | A | C | T | T | T | T | G |
| MLV6-P1 | C/G | T | C | G/A | T | G | C | G/A | T | A | T | C | A | G | C | A | C | T | T | T | T | G |
| MLV6-P2 | G | C/T | T | A | T | G | C | G/A | C/T | A | C/T | C/T | G/A | G | C | A | C | T | T | T | T | G |
| MLV6-P3 | G | C/T | T | A | T | G | C/G | A | T | A | T | C | A | G | C | A | C | T | T | T | T | G |
| MLV6-P4 | G | C/T | C/T | A | T | G | C | G | C/T | A | C/T | C | A | G | C | A | C | T | T | T | T | G |
| MLV7 | C/G | T | C | G/A | T | G | C/G | A | T | G/A | T | C | G/A | G | T | T | C | T | T | C | T | G |

| Animal I.D. | 115 | i193IN | 555 | 150 | 326 | 773 | 804 | 817 | 402 | 408 | 567 | 276 | 494 | 4INDI | 471 | 524 | 478 | 758 | 866 | 950 | 1009 | 1065 | 4476 | 4679 | 4904 |
|---|---|---|---|---|---|---|---|---|---|---|---|---|---|---|---|---|---|---|---|---|---|---|---|---|---|
| MLV7-P1 | G/A | A/T | T | G | T | G | G/A | G | A/C | C/G | C | C | C/T | C | A | T | G | G | C/G | C | G | C | C | A | G/T |
| MLV7-P2 | G/A | A/T | T | G/C | C/T | G | G/A | G | A | C/G | A/C | C | C/T | C | A | T | G/A | C/G | C | C | G/A | C | C | T/A | G |
| MLV7-P3 | A | T | T | G | C/T | G | A | G/A | A/C | C | A/C | C | C/T | C/T | C/A | C/T | G | G | C/G | C | G | C | C | T/A | G |
| MLV7-P4 | A | T | T | G/C | C/T | G | A | G/A | A | C/G | A/C | C | C/T | C/T | C/A | C/T | G | G | C/G | C | G | C | C | T/A | G |
| MLV8 | A | T | T | G/C | C | G | G/A | G/A | A | C/G | C | C | T | C/T | C/A | C/T | G/A | C/G | C/G | C | G/A | C | C/T | T/A | G |
| MLV8-P1 | A | T | T | G/C | C | G | G/A | G | A/C | C/G | C | C | T | C | C/A | T | G/A | C/G | C/G | C | G/A | C | C/T | A | G |
| MLV8-P2 | A | T | T | G/C | C | G | A | G/A | A/C | C | C | C | C/T | C/T | C/A | C/T | G/A | C/G | C | C | G/A | C | C/T | A | G |
| MLV8-P3 | A | T | T | G/C | C/T | G | G/A | G | A/C | C/G | C | C | C/T | C | A | T | G/A | C/G | C | C | G | C | C | T/A | G |
| MLV8-P4 | G/A | A/T | T | G | C | G | A | G/A | A/C | C | C | C | C/T | C/T | C/A | C/T | G/A | C/G | C | C | G/A | C | C/T | A | G |
| MLV9 | G/A | T | T | G/C | C/T | G/A | G/A | G | A | C | A/C | C | T | C/T | C | C/T | G/A | C/G | C/G | C | G/A | C | C | T/A | G |
| MLV9-P1 | G | A/T | T | G/C | C | G | G/A | G | A | C | A/C | C | C/T | C | C/A | T | G/A | C/G | C | C | G/A | C | C | T/A | G |
| MLV9-P2 | G/A | T | T | G/C | C/T | G/A | A | G | A/C | C | C | C | T | C | C | T | G/A | C/G | C/G | C | G | C | C | T | G |
| MLV9-P3 | G | A/T | T | G | C/T | G/A | A | G | A | C | A | C | C/T | C/T | C | C/T | G/A | C/G | C | C | G/A | C | C | A | G |
| MLV9-P4 | G | A/T | T | G | C/T | G/A | A | G | A | C | A | C | C/T | C/T | C | C/T | G/A | C/G | C | C | G | C | C | T/A | G |
| MLV10 | G/A | A/T | T | 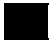 | 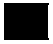 | G | G | G | A | C/G | C | C | C/T | C | C/A | T | G/A | C/G | C/G | C | G | C | C/T | T/A | G |
| MLV10-P1 | G/A | A/T | T | C | C | G | G/A | G | A | C | A/C | C | T | C | C/A | T | G/A | C/G | C/G | C | G/A | C | C | T/A | G |
| MLV10-P2 | G | A | T | G/C | C | G | G/A | G | A/C | C | C | C | C | C/T | C/A | C/T | G/A | C/G | C | C | G | C | C/T | A | G |
| MLV10-P3 | A | T | T | G | C/T | G | G/A | G | A/C | C | C | C | C/T | C | C | T | G | G | G | C | G | C | C/T | A | G |
| MLV10-P4 | A | T | T | G/C | C | G | G/A | G | A/C | C | C | C | C/T | C | C | T | G | G | G | C | G | C | C/T | A | G |
| MLV11 | G/A | A/T | T | G/C | C/T | G | A | G/A | A | C | A/C | C | C/T | C | C/A | T | G/A | C/G | C/G | C | G/A | C | C | T/A | G |
| MLV11-P1 | A | T | T | G/C | C | G | A | G | A | C/G | A/C | C | C/T | C | C/A | T | G | G | C/G | C | G/A | C | C | T/A | G |
| MLV11-P2 | A | T | T | G | C/T | G | G/A | G | A | C/G | A/C | C | T | C/T | C/A | C/T | G/A | C/G | C/G | C | A | C | C | T/A | G |
| MLV11-P3 | G/A | A/T | T | G | C/T | G | A | G/A | A | C/G | A/C | C | T | C | A | T | A | C | C | C | G | C | C | A | G |
| MLV11-P4 | G/A | A/T | T | G/C | C | G | G/A | G | A | C/G | C | C | T | C | A | T | G/A | C/G | C | C | G | C | C | A | G |
| MLV12 | G/A | A/T | T | G/C | C/T | G | G/A | G/A | A | C | A/C | C | T | C/T | C/A | C/T | G/A | C/G | C/G | C | G | C | C/T | T/A | G |
| MLV12-P1 | G/A | A/T | T | G/C | C | G | A | G/A | A | C/G | C | C | T | C | A | T | G/A | C/G | C/G | C | G | C | C | T | G |
| MLV12-P2 | A | T | T | G | C/T | G | A | G/A | A | C | C | C | C/T | C/T | C/A | C/T | G | G | C/G | C | G/A | C | C/T | A | G |
| MLV12-P3 | A | T | T | G/C | C | G | A | G/A | A | C/G | C | C | C/T | C/T | C/A | C/T | G | G | C/G | C | G/A | C | C | T/A | G |
| MLV12-P4 | G/A | A/T | T | G/C | C | G | G/A | G | A | C/G | A/C | C | T | C | A | T | A | C | C | C | G | C | C | T/A | G |
| MLV13 | G/A | A/T | T | G | C/T | G | G/A | G/A | A/C | C | C | C | T | C | C/A | T | G/A | C/G | C | C | G/A | C | C | T/A | G/T |
| MLV13-P1 | A | T | T | G | C | G | A | G/A | A | C/G | C | C | C/T | C | C/A | T | G | G | C | C | G | C | C | A | G/T |
| MLV13-P2 | G/A | A/T | T | G | C/T | G | A | G/A | A/C | C | C | C | T | C | A | T | G/A | C/G | C | C | G/A | C | C | A | G/T |
| MLV13-P3 | A | T | T | G | C | G | G/A | G | A | C | C | C | T | C | C/A | T | G/A | C/G | C | C | A | C | C | A | G/T |
| MLV13-P4 | A | T | T | G | C | G | A | G/A | A | C/G | C | C | C/T | C | C/A | T | G | G | C | C | A | C | C | A | G/T |

| Animal I.D. | 419 | 515 | 184 | 389 | 516 | 582 | 426 | 810 | 368 | 533 | 939 | 71 | 138 | 361 | 15543 | 15558 | 15615 | 15616 | 15675 | 15714 | 15840 | 16127 |
|---|---|---|---|---|---|---|---|---|---|---|---|---|---|---|---|---|---|---|---|---|---|---|
| MLV7-P1 | G | C/T | C/T | A | T | G | G | A | T | A | T | C | G | G/A | T | T | C | T | T | C | T | G |
| MLV7-P2 | G | T | C | A | T | G | C | G/A | T | G | T | C | G | G | T | T | C | T | T | C | T | G |
| MLV7-P3 | C/G | T | C | G/A | T | G | C/G | A | T | G/A | T | C | G/A | G | T | T | C | T | T | C | T | G |
| MLV7-P4 | G | T | C | A | T | G | C | G/A | T | G | T | C | G/A | G/A | T | T | C | T | T | C | T | G |
| MLV8 | C/G | T | C | G/A | T | G | C | A | T | A | T | T | G | G | T | T | C | T | T | C | T | G |
| MLV8-P1 | C/G | C/T | C | G/A | T | G/A | C/G | A | T | G/A | T | C/T | G | G | T | T | C | T | T | C | T | G |
| MLV8-P2 | G | C/T | C/T | A | T | G | C | G/A | T | A | T | C/T | G | G/A | T | T | C | T | T | C | T | G |
| MLV8-P3 | C/G | C/T | C/T | G/A | T | G | C/G | A | T | A | T | C/T | G | G/A | T | T | C | T | T | C | T | G |
| MLV8-P4 | C/G | T | C | G/A | T | G | C/G | A | T | G/A | T | C/T | G | G/A | T | T | C | T | T | C | T | G |
| MLV9 | G | C | C/T | A | T | G/A | C/G | A | T | A | T | T | G | G | C | A | C | T | T | T | T | G |
| MLV9-P1 | G | C | C/T | A | T | G/A | C/G | G/A | T | A | T | C/T | G | G | C | A | C | T | T | T | T | G |
| MLV9-P2 | G | C | C/T | A | T | G/A | G | A | T | A | T | C/T | G | G | C | A | C | T | T | T | T | G |
| MLV9-P3 | G | C/T | C/T | A | T | G | C | G/A | T | G/A | T | C/T | G | G/A | C | A | C | T | T | T | T | G |
| MLV9-P4 | G | C | T | A | T | G | C/G | A | T | A | T | C/T | G | G/A | C | A | C | T | T | T | T | G |
| MLV10 | C | T | C | A | T | A | C/G | A | T | A | T | C | G/A | G | T | T | C | T | T | C | T | G |
| MLV10-P1 | C/G | C/T | C | A | T | A | C | A | T | G/A | T | C | G | G | T | T | C | T | T | C | T | G |
| MLV10-P2 | C/G | C/T | C/T | A | T | G/A | G | A | T | G/A | T | C | G/A | G/A | T | T | C | T | T | C | T | G |
| MLV10-P3 | C/G | C/T | C | A | T | A | C | A | T | G/A | T | C | G/A | G | T | T | C | T | T | C | T | G |
| MLV10-P4 | C/G | C/T | C | A | T | A | C/G | A | T | A | T | C | G/A | G | T | T | C | T | T | C | T | G |
| MLV11 | C/G | T | C | G/A | T | G | C/G | A | C/T | A | T | C | G | G/A | T | T | C | T | T | C | T | G |
| MLV11-P1 | G | T | C | A | T | G/A | G | A | T | G/A | T | C/T | G | G | T | T | C | T | T | C | T | G |
| MLV11-P2 | G | T | C | A | T | A | G | A | T | A | T | C | G/A | G | C | A | C | T | C | C | C | G |
| MLV11-P3 | G | T | C | A | T | G/A | C | C/A | C/T | G/A | T | C | G/A | G | T | T | C | T | T | C | T | G |
| MLV11-P4 | G | T | C | A | T | A | C/G | A | T | A | T | C | A | G | C | A | C | T | C | C | C | G |
| MLV12 | G | C/T | C/T | A | T | G | C/G | A | C/T | A | T | C | G/A | G | C | A | C | T | C | T | T | A |
| MLV12-P1 | G | T | C | A | T | G/A | C/G | A | C/T | G/A | T | C | A | G | C | A | C | T | C | T | T | A |
| MLV12-P2 | G | C/T | A | T | G/A | G | A | T | G/A | T | C | G/A | G | C | A | C | T | C | T | T | A | |
| MLV12-P3 | G | C/T | C/T | A | T | G/A | C/G | C/A | T | G/A | T | C | A | G | C | A | C | T | C | T | T | A |
| MLV12-P4 | G | T | C | A | T | A | C | C/A | T | G/A | T | C | A | G | C | A | C | T | C | C | C | G |
| MLV13 | C/G | T | C | G/A | T | G | C/G | A | T | A | T | C | A | G | C | A | C | T | T | T | T | G |
| MLV13-P1 | C/G | T | C | G/A | T | G/A | C/G | C/A | T | G/A | T | C | A | G | C | A | C | T | T | T | T | G |
| MLV13-P2 | C/G | T | C | G/A | T | G/A | C/G | A | T | G/A | T | C | A | G | C | A | C | T | T | T | T | G |
| MLV13-P3 | G | T | C | A | T | G/A | C/G | C/A | T | G/A | T | C | A | G | C | A | C | T | T | T | T | G |
| MLV13-P4 | C/G | T | C | G/A | T | G/A | C | C/A | T | G/A | T | C | A | G | C | A | C | T | T | T | T | G |

| Animal I.D. | 115 | i193IN | 555 | 150 | 326 | 773 | 804 | 817 | 402 | 408 | 567 | 276 | 494 | 4INDI | 471 | 524 | 478 | 758 | 866 | 950 | 1009 | 1065 | 4476 | 4679 | 4904 |
|---|---|---|---|---|---|---|---|---|---|---|---|---|---|---|---|---|---|---|---|---|---|---|---|---|---|
| MLV14 | G | A/T | T | G | C | G | G | G | A | C/G | A/C | C | T | C | C/A | C/T | A | C | C | C/T | G/A | C/T | C | T/A | G |
| MLV14-P1 | G/A | T | T | G | C | G | G/A | G | A | G | C | C | C/T | C | C/A | C/T | G/A | C/G | C | C/T | G | C/T | C | T/A | G |
| MLV14-P2 | F | F | F | G | C | G | G/A | G | A | G | C | C | C/T | C | C/A | C/T | G/A | C/G | C | C/T | G/A | C/T | C | A | G |
| MLV14-P3 | G/A | A/T | T | G | C | G | G/A | G | A | C | A/C | C | C/T | C | A | T | G/A | C/G | C | C | A | C | C | A | G |
| MLV14-P4 | G/A | A/T | T | G | C | G | G/A | G | A/C | C/G | A/C | C | C/T | C | A | T | G/A | C/G | C | C | G/A | C | C/T | T/A | G |
| MLV15 | G/A | A/T | T | G | C/T | G | G/A | G/A | A | C | A/C | C | T | C/T | C | C/T | G/A | C/G | C/G | C | G/A | C | C | T/A | G |
| MLV15-P1 | A | T | T | G | C | G | A | G/A | A | C | C | C | C/T | C/T | C/A | C/T | G | G | C/G | C | G | C | C | A | G |
| MLV15-P2 | G/A | A/T | T | G | C | G | A | G/A | A | C | C | C | T | C | C/A | T | G | G | C/G | C | A | C | C | T/A | G |
| MLV15-P3 | A | T | T | G | C/T | G | A | G/A | A | C/G | A/C | C | C/T | C/T | C/A | C/T | G | G | C/G | C | G | C | C | T/A | G |
| MLV15-P4 | A | T | T | G | C/T | G | A | G/A | A | C/G | C | C | C/T | C/T | C/A | C/T | G | G | C/G | C | G | C | C | T/A | G |
| MLV16 | G | A/T | T | G/C | C/T | G | G | G | A | C/G | A/C | C | T | C | C/A | T | G/A | C/G | C/G | C | G | C | C/T | A | G/T |
| MLV16-P1 | G/A | T | T | G/C | C | G | G/A | G | A | C/G | A/C | C | T | C | A | T | G/A | C/G | C | C | G/A | C | C | A | G/T |
| MLV16-P2 | G/A | A/T | T | G | C/T | G | G/A | G | A | C/G | A/C | C | C/T | C | A | T | G/A | C/G | C | C | G/A | C | C/T | A | G |
| MLV16-P3 | G/A | T | T | G | C/T | G | G/A | G | A | C/G | C | C | C/T | C | C/A | T | G | G | C/G | C | G/A | C | C/T | A | G |
| 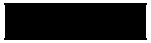 | 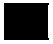 | 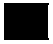 | 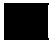 | 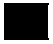 | 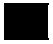 | 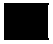 | 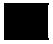 | 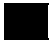 | 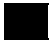 | 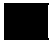 | 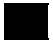 | 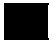 | 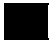 | 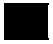 | 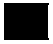 | 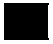 | 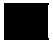 | 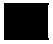 | 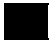 | 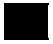 | 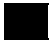 | 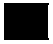 | 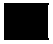 | 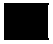 | 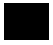 |
| MLV17 | A | T | T | G | C | G | G/A | G/A | A | C | A/C | C | C/T | C | C/A | T | G/A | C/G | C/G | C | G | C | C | T/A | G |
| MLV17-P1 | A | T | T | G | C | G | A | G/A | A | C/G | A/C | C | C/T | C | A | T | G | G | C/G | C | G | C | C | T/A | G |
| MLV17-P2 | A | T | T | G | C | G | G/A | G | A | C | C | C | T | C | C/A | T | G | G | C/G | C | G | C | C | T/A | G |
| MLV17-P3 | G/A | T | T | G | C | G | G/A | G | A | C | C | C | T | C | C/A | T | G | G | C/G | C | G | C | C | A | G |
| MLV17-P4 | A | T | T | G | C | G | A | G/A | A | C/G | C | C | T | C | A | T | G/A | C/G | C | C | G/A | C | C | A | G |
| MLV18 | G | A/T | T | G/C | C | G | G/A | G | A | C/G | C | C | T | C | C | C/T | G/A | C/G | C | C/T | G/A | C/T | C/T | T/A | G |
| MLV18-P1 | G/A | T | T | G/C | C | G | A | G | A | C | C | C | T | C | C/A | C/T | G/A | C/G | C | C/T | G/A | C/T | C | T/A | G |
| MLV18-P2 | G/A | T | T | G/C | C | G | G/A | G | A | C/G | C | C | C/T | C | C/A | C/T | G | G | C | C/T | G/A | C/T | C/T | A | G |
| MLV18-P3 | G/A | T | T | G | C | G | G/A | G | A | C/G | C | C | C/T | C | C/A | C/T | G | G | C | C/T | G/A | C/T | C/T | A | G |
| MLV18-P4 | G/A | T | T | G/C | C | G | A | G | A | G | C | C | C/T | C | C/A | C/T | G/A | C/G | C | C/T | G/A | C/T | C/T | A | G |
| MLV19 | G/A | A/T | T | G | C | G | G | G | A/C | C | C | C | T | C | C/A | T | A | C | C | C | G | C | C/T | T/A | G |
| MLV19-P1 | G/A | A/T | T | G | C | G | G/A | G | A | C | C | C | T | C | A | T | A | C | C | C | G | C | C | T | G |
| MLV19-P2 | G/A | A/T | T | G | C | G | G/A | G | A/C | C/G | C | C | C/T | C | A | T | G/A | C/G | C | C | G | C | C/T | A | G |
| MLV19-P3 | G/A | A/T | T | G | C | G | G/A | G | A/C | C/G | C | C | T | C | A | T | G/A | C/G | C | C | G/A | C | C | T/A | G |
| MLV19-P4 | A | T | T | G | C | G | G/A | G | A/C | C | C | C | T | C | C/A | T | A | C | C | C | G | C | C/T | A | G |
| MLV20 | A | T | T | G/C | C | G | G | G | A | C | A/C | C | T | C | C | C/T | G | C/G | C | C | G/A | C | C | T/A | G |
| MLV20-P1 | A | T | T | G/C | C | G | G/A | G | A | C | C | C | C/T | C | C/A | C/T | G | C/G | C | C | G/A | C | C | A | G |
| MLV20-P2 | A | T | T | G/C | C | G | G/A | G | A | C/G | A/C | C | T | C | C/A | T | G/A | C/G | C | C | A | C | C | T/A | G |

| Animal I.D. | 419 | 515 | 184 | 389 | 516 | 582 | 426 | 810 | 368 | 533 | 939 | 71 | 138 | 361 | 15543 | 15558 | 15615 | 15616 | 15675 | 15714 | 15840 | 16127 |
|---|---|---|---|---|---|---|---|---|---|---|---|---|---|---|---|---|---|---|---|---|---|---|
| MLV14 | G | C/T | C/T | A | T | G | C/G | A | C/T | A | T | T | G | G | T | T | C | T | T | C | T | G |
| MLV14-P1 | G | T | C | A | T | G/A | C/G | C/A | T | G/A | T | C/T | G/A | G | T | T | C | T | T | C | T | G |
| MLV14-P2 | G | C/T | C/T | A | T | G/A | F | F | 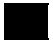 | 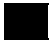 | 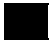 | C/T | G/A | G | T | T | C | T | T | C | T | G |
| MLV14-P3 | G | T | C | A | T | G/A | C/G | C/A | C/T | G/A | T | C/T | G/A | G | T | T | C | T | T | C | T | G |
| MLV14-P4 | G | C/T | C/T | A | T | G/A | C | C/A | C/T | G/A | T | F | F | F | T | T | C | T | T | C | T | G |
| MLV15 | C/G | C/T | C | A | T | A | C/G | G/A | C/T | G/A | T | C/T | G/A | G | T | T | C | T | T | C | T | G |
| MLV15-P1 | C/G | T | C | A | T | A | G | A | T | G | T | C | A | G | T | T | C | T | T | C | T | G |
| MLV15-P2 | G | C/T | C | A | T | A | G | A | T | G | T | C | A | G | T | T | C | T | T | C | T | G |
| MLV15-P3 | C/G | T | C | A | T | A | C/G | G/A | T | G | T | C/T | G/A | G | T | T | C | T | T | C | T | G |
| MLV15-P4 | C/G | T | C | A | T | A | C/G | C/A | T | G | T | C/T | G/A | G | T | T | C | T | T | C | T | G |
| MLV16 | C/G | T | C | A | T | G | C/G | A | C/T | G/A | T | C | G/A | G | C | A | C | T | C | C | C | G |
| MLV16-P1 | C/G | T | C | A | T | A | C | C/A | C/T | G/A | T | C | G/A | G | C | A | C | T | C | C | C | G |
| MLV16-P2 | C/G | T | C | A | T | A | C | C/A | C/T | G/A | T | C | A | G | C | A | C | T | C | C | C | G |
| MLV16-P3 | G | T | C | A | T | G/A | G | A | T | G | T | C | G/A | G | C | A | C | T | C | C | C | G |
| 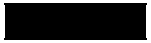 | 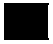 | 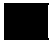 | 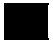 | 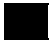 | 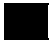 | 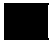 | 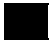 | 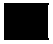 | 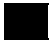 | 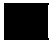 | 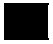 | 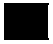 | 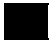 | 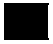 | F | F | F | F | F | F | F | F |
| MLV17 | G | T | C/T | A | T | G | C/G | A | C/T | A | T | C/T | G | G | C | A | C | T | C | C | C | G |
| MLV17-P1 | G | T | C/T | A | T | G/A | C/G | A | C/T | G/A | T | C/T | G/A | G | C | A | C | T | C | C | C | G |
| MLV17-P2 | G | T | C | A | T | G/A | C/G | C/A | C/T | G/A | T | C | G/A | G | C | A | C | T | C | C | C | G |
| MLV17-P3 | C/G | T | C | A | C/T | G/A | F | F | 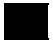 | 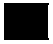 | 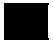 | C/T | G/A | G | C | A | C | T | C | C | C | G |
| MLV17-P4 | G | T | C | A | T | G/A | C/G | C/A | T | G/A | T | C/T | G/A | G | C | A | C | T | C | C | C | G |
| MLV18 | G | C/T | C/T | A | T | G | C/G | G/A | C/T | G/A | T | C | G/A | G | T | T | C | T | T | C | T | G |
| MLV18-P1 | G | T | C | A | T | G/A | C/G | G/A | C/T | G/A | T | C | G/A | G | T | T | C | T | T | C | T | G |
| MLV18-P2 | G | C/T | C/T | A | T | G/A | C/G | C/A | C/T | G/A | T | C | A | G | T | T | C | T | T | C | T | G |
| MLV18-P3 | G | T | C | A | T | G/A | C/G | G/A | C/T | G/A | T | C | G/A | G | T | T | C | T | T | C | T | G |
| MLV18-P4 | G | T | C | A | T | G/A | C | C/G | T | G | T | C | A | G | T | T | C | T | T | C | T | G |
| MLV19 | G | C/T | C/T | A | T | G | C/G | A | C/T | A | T | C | G/A | G/A | T | T | C | T | T | C | T | G |
| MLV19-P1 | G | T | C | A | T | G/A | G | A | T | G/A | T | C | G/A | G/A | T | T | C | T | T | C | T | G |
| MLV19-P2 | G | C/T | C/T | A | T | G/A | C | C/A | C/T | G/A | T | C | A | G | T | T | C | T | T | C | T | G |
| MLV19-P3 | G | T | C | A | T | G/A | C/G | A | T | G/A | T | C | G/A | G/A | T | T | C | T | T | C | T | G |
| MLV19-P4 | G | T | C | A | T | G/A | C/G | A | C/T | A | T | C | A | G | T | T | C | T | T | C | T | G |
| MLV20 | G | C/T | C/T | A | T | G | C/G | G/A | C/T | A | T | T | G | G | T | T | C | T | T | C | T | G |
| MLV20-P1 | G | C/T | C/T | A | T | G/A | C/G | C/A | T | G/A | T | C/T | G/A | G | T | T | C | T | T | C | T | G |
| MLV20-P2 | G | T | C | A | T | G/A | C/G | G/A | T | A | T | C/T | G/A | G | T | T | C | T | T | C | T | G |

| Animal I.D. | 115 | i193IN | 555 | 150 | 326 | 773 | 804 | 817 | 402 | 408 | 567 | 276 | 494 | 4INDI | 471 | 524 | 478 | 758 | 866 | 950 | 1009 | 1065 | 4476 | 4679 | 4904 |
|---|---|---|---|---|---|---|---|---|---|---|---|---|---|---|---|---|---|---|---|---|---|---|---|---|---|
| MLV20-P3 | A | T | T | G | C | G | G/A | G | A | C | C | C | T | C | C/A | C/T | G | G | C | C | G | C | C | T/A | G |
| MLV20-P4 | A | T | T | G | C | G | G/A | G | A | C | A/C | C | T | C | C/A | T | G | G | C | C | G/A | C | C | A | G |
| MLV21 | G/A | A/T | T | G | C/T | G | G | G | A/C | C | C | C | T | C/T | C | C/T | G/A | C/G | C/G | C/T | G/A | C/T | C | T/A | G |
| 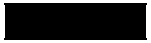 | 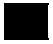 | 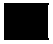 | 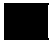 | 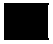 | 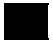 | 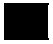 | 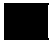 | 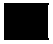 | 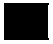 | 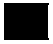 | 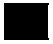 | 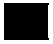 | 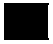 | 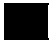 | 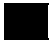 | 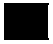 | 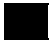 | 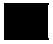 | 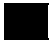 | 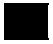 | 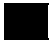 | 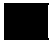 | 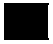 | 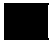 | 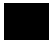 |
| MLV21-P2 | G/A | A/T | T | G | C/T | G | G/A | G | A | C | C | C | T | C | C/A | T | G | G | C/G | C/T | G | C/T | C | A | G |
| MLV21-P3 | G/A | A/T | T | G | C/T | G | G/A | G | A | C | A/C | C | T | C/T | C/A | C/T | G | G | C/G | C/T | G | C/T | C | T/A | G |
| MLV21-P4 | A | T | T | G | C/T | G | G/A | G | A | C | C | C | T | C/T | C/A | C/T | G | G | C/G | C/T | G | C/T | C | T/A | G/T |
| MLV22 | G/A | A/T | T | G | C/T | G | A | A | A/C | C/G | C | C | C/T | C/T | C/A | C/T | G | C/G | C/G | C | G | C | C/T | T/A | G |
| MLV22-P1 | G | A | T | G | C/T | G | A | G/A | A | C/G | C | C | C/T | C | C/A | T | G | G | C/G | C | G | C | C/T | A | G |
| 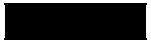 | 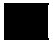 | 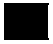 | 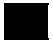 | 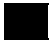 | 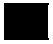 | 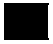 | 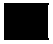 | 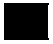 | 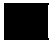 | 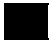 | 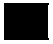 | 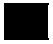 | 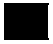 | 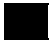 | 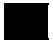 | 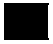 | 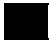 | 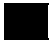 | 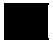 | 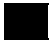 | 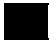 | 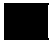 | 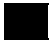 | 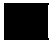 | 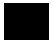 |
| MLV22-P3 | G/A | A/T | T | G | C/T | G | A | G/A | A/C | C | C | C | C/T | C/T | C | C/T | G | C/G | C | C | G | C | C | T/A | G |
| MLV22-P4 | G | A | T | G | C | G | A | G/A | A | C/G | A/C | C | C/T | C | C/A | T | G | C/G | C | C | G | C | C | T/A | G |
| MLV24 | G | T | T | G | C | G | G | G | A/C | C | C | C | T | C | C/A | T | G/A | C | C | C | G/A | C | C | T/A | G |
| MLV24-P1 | G | A/T | T | G | C | G | G/A | G | A | C | C | C | T | C | C | T | G/A | C/G | C | C | G/A | C | C | T/A | G |
| MLV24-P2 | G/A | T | T | G | C | G | G/A | G | A | C | C | C | T | C | C/A | T | G | C/G | C | C | G | C | C | A | G |
| MLV24-P3 | G/A | T | T | G | C | G | G/A | G | A/C | C | C | C | T | C | A | T | G | C/G | C | C | G/A | C | C | A | G |
| MLV24-P4 | G | A/T | T | G | C | G | G/A | G | A | C | C | C | T | C | C | T | G/A | C/G | C | C | G | C | C | A | G |
| MLV25 | A | T | T | G/C | C | G | G | G | A | C | A/C | C | T | C/T | C/A | C/T | G/A | C/G | C/G | C/T | G | C/T | C | T/A | G |
| MLV25-P1 | A | T | T | G/C | C | G | G/A | G/A | A | C/G | A/C | C | T | C/T | C/A | C/T | G | G | C/G | C/T | G | C/T | C | A | G/T |
| MLV25-P2 | G/A | A/T | T | G | C | G | G/A | G/A | A | C/G | C | C | T | C/T | C/A | C/T | G | G | C/G | C/T | G | C/T | C | A | G/T |
| MLV25-P3 | G/A | A/T | T | G/C | C | G | G/A | G | A | C/G | C | C | T | C | A | T | G/A | C/G | C | C | G | C | C | T/A | G/T |
| MLV25-P4 | G/A | A/T | T | G/C | C | G | G/A | G/A | A | C/G | A/C | C | T | C/T | C/A | C/T | G | G | C/G | C/T | G | C/T | C | A | G |
| MLV26 | G/A | A/T | T | G | C/T | G | G/A | G | A | C | A | C | T | C | A | T | G | C/G | C/G | C | G | C | C | T/A | G/T |
| MLV26-P1 | G/A | A/T | T | G | C/T | G | A | G | A | C | A | C | T | C | A | T | G | G | C/G | C | G | C | C | T/A | G |
| MLV26-P2 | G | A | T | G | C | G | A | G | A | C | A/C | C | T | C | C/A | C/T | G | C/G | C | C | G | C | C | A | G/T |
| MLV26-P3 | G/A | A/T | T | G | C | G | A | G | A | C | A | C | T | C | A | T | G | G | C/G | C | G | C | C | T/A | G |
| MLV26-P4 | G/A | A/T | T | G | C | G | A | G/A | A | C/G | A/C | C | T | C | A | T | G | C/G | C | C | G | C | C | A | G/T |
| MLV27 | G/A | A/T | T | G/C | C | G | G/A | G/A | A/C | C | C | C | T | C/T | C/A | C/T | G/A | C/G | C/G | C | G/A | C | C | T/A | G |
| MLV27-P1 | G | A | T | G | C | G | G/A | G | A | C/G | C | C | T | C | A | T | G/A | C/G | C | C | G/A | C | C | A | G/T |
| MLV27-P2 | A | T | T | G/C | C | G | G/A | G/A | A/C | C/G | C | C | T | C/T | C/A | C/T | G | G | C/G | C | G | C | C | A | G |
| MLV27-P3 | G/A | A/T | T | G/C | C | G | A | A | A | C/G | C | C | T | C/T | C/A | C/T | G | G | C/G | C | G | C | C | A | G |
| MLV27-P4 | G/A | A/T | T | G | C | G | G/A | G | A | C/G | C | C | T | C | A | T | G/A | C/G | C | C | G/A | C | C | A | G |
| MLV28 | G/A | A/T | T | G/C | C/T | G | G | G | A/C | C | C | C | T | C | C/A | T | A | C | C | C | G | C | C/T | T/A | G |

| Animal I.D. | 419 | 515 | 184 | 389 | 516 | 582 | 426 | 810 | 368 | 533 | 939 | 71 | 138 | 361 | 15543 | 15558 | 15615 | 15616 | 15675 | 15714 | 15840 | 16127 |
|---|---|---|---|---|---|---|---|---|---|---|---|---|---|---|---|---|---|---|---|---|---|---|
| MLV20-P3 | G | C/T | C/T | A | T | G/A | C | C/G | C/T | G/A | T | C/T | G/A | G | T | T | C | T | T | C | T | G |
| MLV20-P4 | G | T | C | A | T | G/A | C/G | G/A | C/T | G/A | T | C/T | G/A | G | T | T | C | T | T | C | T | G |
| MLV21 | G | C | C/T | A | T | G/A | C/G | A | C/T | A | T | T | G | G | T | T | C | T | T | C | T | G |
| 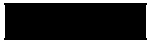 | 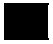 | 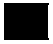 | 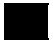 | 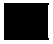 | 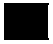 | 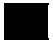 | 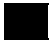 | 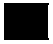 | 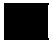 | 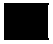 | 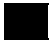 | 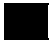 | 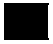 | 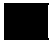 | F | F | F | F | F | F | F | F |
| MLV21-P2 | G | C | C | A | T | A | C/G | C/A | T | G/A | T | C/T | G/A | G | T | T | C | T | T | C | T | G |
| MLV21-P3 | G | C/T | C/T | A | T | G/A | G | A | T | G/A | T | T | G | G | T | T | C | T | T | C | T | G |
| MLV21-P4 | G | C | C | A | T | A | G | A | C/T | G/A | T | C/T | G/A | G | T | T | C | T | T | C | T | G |
| MLV22 | G | T | C/T | A | T | G | C/G | A | T | A | T | C/T | G | G | C | A | C | T | C | T | T | A |
| MLV22-P1 | G | C/T | C | A | T | G/A | C/G | C/A | T | G/A | T | C/T | G/A | G | C | A | C | T | C | T | T | A |
| 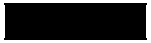 | 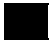 | 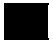 | 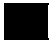 | 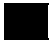 | 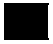 | 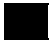 | 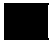 | 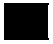 | 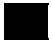 | 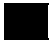 | 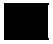 | 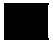 | 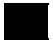 | 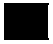 | F | F | F | F | F | F | F | F |
| MLV22-P3 | G | C/T | F | F | F | F | C/G | C/A | T | G/A | T | C | G/A | G | C | A | C | T | C | T | T | A |
| MLV22-P4 | G | C/T | C | A | T | G/A | C | C/A | T | A | T | C | G/A | G | C | A | C | T | C | T | T | A |
| MLV24 | C/G | C/T | C/T | A | T | G/A | C/G | A | T | G | T | T | G | G | C | A | C | T | T | T | T | G |
| MLV24-P1 | G | C | C/T | A | T | G/A | C/G | A | T | G | T | T | G | G | C | A | C | T | T | T | T | G |
| MLV24-P2 | G | C/T | C/T | A | T | G/A | G | A | T | G | T | T | G | G | C | A | C | T | T | T | T | G |
| MLV24-P3 | G | C | C/T | A | T | G/A | C/G | A | T | G | T | T | G | G | C | A | C | T | T | T | T | G |
| MLV24-P4 | G | C | C/T | A | T | G/A | G | A | T | G | T | C/T | G/A | G | C | A | C | T | T | T | T | G |
| MLV25 | G | T | C/T | A | T | G | C/G | A | T | A | T | C | A | G | T | T | C | T | T | C | T | G |
| MLV25-P1 | G | T | C/T | A | T | G/A | C/G | A | T | A | T | C | A | G | T | T | C | T | T | C | T | G |
| MLV25-P2 | G | C/T | C/T | A | T | G/A | C/G | C/A | T | A | T | C | A | G | T | T | C | T | T | C | T | G |
| MLV25-P3 | G | T | C | A | T | G/A | C/G | C/A | T | G/A | T | C | A | G | T | T | C | T | T | C | T | G |
| MLV25-P4 | G | T | C | A | T | G/A | C | C/A | T | G/A | T | C | G/A | G | T | T | C | T | T | C | T | G |
| MLV26 | G | C | C/T | A | T | G/A | C/G | A | T | A | T | C | A | G | T | T | C | T | T | C | T | G |
| MLV26-P1 | G | C/T | C | A | T | A | G | A | T | G/A | T | C | A | G | T | T | C | T | T | C | T | G |
| MLV26-P2 | G | C/T | C/T | A | T | G/A | G | A | T | G/A | T | C | A | G | T | T | C | T | T | C | T | G |
| MLV26-P3 | G | C/T | C/T | A | T | G/A | C/G | A | T | G/A | T | C | A | G | T | T | C | T | T | C | T | G |
| MLV26-P4 | G | C/T | C | A | T | A | C/G | A | T | A | T | C | G/A | G | T | T | C | T | T | C | T | G |
| MLV27 | G | C/T | C/T | A | T | G | C/G | A | C/T | A | T | C/T | G | G | T | T | C | T | T | C | T | G |
| MLV27-P1 | G | C | C/T | A | T | G/A | C | C/A | C/T | G/A | T | C/T | G/A | G | T | T | C | T | T | C | T | G |
| MLV27-P2 | G | C | C/T | A | T | G/A | C/G | A | C/T | G/A | T | C | G | G | T | T | C | T | T | C | T | G |
| MLV27-P3 | G | C | C/T | A | T | G/A | G | A | T | A | T | C/T | G | G | T | T | C | T | T | C | T | G |
| MLV27-P4 | G | C/T | C | A | T | G/A | G | A | C/T | G/A | T | C/T | G | G | T | T | C | T | T | C | T | G |
| MLV28 | G | C | C/T | A | T | G/A | C | C/A | T | G/A | T | T | G | G | C | A | C | T | C | C | C | G |

| Animal I.D. | 115 | i193IN | 555 | 150 | 326 | 773 | 804 | 817 | 402 | 408 | 567 | 276 | 494 | 4INDI | 471 | 524 | 478 | 758 | 866 | 950 | 1009 | 1065 | 4476 | 4679 | 4904 |
|---|---|---|---|---|---|---|---|---|---|---|---|---|---|---|---|---|---|---|---|---|---|---|---|---|---|
| MLV28-P1 | G/A | A/T | T | G | C/T | G | G/A | G/A | A | C/G | C | C | T | C | A | T | G/A | C/G | C | C | G | C | C | T/A | G/T |
| MLV28-P2 | G/A | A/T | T | G | C/T | G | G/A | G | A/C | C/G | C | C | T | C | C/A | T | G/A | C/G | C | C | G | C | C | T/A | G/T |
| MLV28-P3 | G/A | A/T | T | G/C | C | G | G/A | G/A | A | C/G | C | C | T | C | C/A | T | G/A | C/G | C | C | G | C | C/T | A | G |
| MLV28-P4 | G/A | A/T | T | G | C/T | G | G/A | G/A | A | C/G | C | C | T | C | C/A | T | G/A | C/G | C | C | G | C | C | T/A | G |
| MLV29 | G/A | A/T | T | G/C | C | G | G/A | G/A | A | C/G | A/C | C | T | C/T | C | C/T | G/A | C/G | C/G | C | G | C | C/T | T/A | G |
| MLV29-P1 | G/A | A/T | T | G | C | G | G/A | G | A | C/G | C | C | T | C | C/A | T | A | C | C | C | G | C | C | T/A | G |
| MLV29-P2 | G | A | T | G | C | G | A | G/A | A | C | A | C | T | C | C | C/T | G/A | C/G | C | C | G | C | C | T/A | G |
| MLV29-P3 | G | A | T | G/C | C | G | A | G/A | A | C | A/C | C | T | C | C/A | T | G/A | C/G | C | C | G | C | C/T | A | G/T |
| MLV29-P4 | G/A | A/T | T | G/C | C | G | G/A | G/A | A | C/G | C | C | T | C | C/A | T | G/A | C/G | C | C | G | C | C | T/A | G |
| MLV30 | G/A | T | T | G/C | C/T | G | G/A | G | A/C | C/G | C | C | T | C/T | C/A | C/T | G | G | C/G | C | G/A | C | C/T | T/A | G |
| MLV30-P1 | G/A | A/T | T | G/C | C | G | G/A | G | A | C/G | C | C | T | C/T | C | C | G | G | C/G | C | G | C | C/T | A | G |
| MLV30-P2 | G/A | A/T | T | G | C/T | G | G/A | G | A | C/G | A/C | C | T | C/T | C/A | C/T | G | G | C/G | C | G | C | C | T/A | G |
| MLV30-P3 | G/A | A/T | T | G/C | C | G | A | G | A | C/G | C | C | T | C/T | C | C | G | G | C/G | C | G | C | C/T | A | G |
| MLV30-P4 | G | A/T | T | G | C/T | G | G/A | G | A | C/G | A/C | C | T | C | C/A | C/T | G | G | C/G | C | G/A | C | C/T | A | G |

| Animal I.D. | 419 | 515 | 184 | 389 | 516 | 582 | 426 | 810 | 368 | 533 | 939 | 71 | 138 | 361 | 15543 | 15558 | 15615 | 15616 | 15675 | 15714 | 15840 | 16127 |
|---|---|---|---|---|---|---|---|---|---|---|---|---|---|---|---|---|---|---|---|---|---|---|
| MLV28-P1 | G | C/T | C/T | A | T | G/A | C/G | C/A | T | G | T | C/T | G | G | C | A | C | T | C | C | C | G |
| MLV28-P2 | G | C | C/T | A | T | G/A | C | C/A | T | A | T | C/T | G/A | G | C | A | C | T | C | C | C | G |
| MLV28-P3 | G | C/T | C/T | A | T | G/A | C/G | C/A | T | G | T | C/T | G | G | C | A | C | T | C | C | C | G |
| MLV28-P4 | G | C | C | A | T | A | C/G | C/A | T | A | T | C/T | G | G | C | A | C | T | C | C | C | G |
| MLV29 | C/G | T | C | G/A | T | G | C/G | A | T | A | T | C | G | G/A | C | A | C | T | C | C | C | G |
| MLV29-P1 | C/G | C/T | C | G/A | T | G/A | C/G | C/A | T | G/A | T | C | G | G/A | C | A | C | T | C | C | C | G |
| MLV29-P2 | G | C/T | C | A | T | G/A | C/G | A | T | G/A | T | C | G | G | C | A | C | T | C | C | C | G |
| MLV29-P3 | G | C/T | C | A | T | G/A | C | C/A | T | G/A | T | C | G/A | G/A | C | A | C | T | C | C | C | G |
| MLV29-P4 | G | T | C | A | T | G/A | C/G | C/A | T | G/A | T | C | G/A | G | C | A | C | T | C | C | C | G |
| MLV30 | C/G | T | C | G/A | T | G | C/G | A | T | G/A | T | C/T | G | G | T | T | C | T | T | C | T | G |
| MLV30-P1 | G | T | C | A | T | G/A | G | A | T | G | T | C/T | G/A | G | T | T | C | T | T | C | T | G |
| MLV30-P2 | C/G | T | C | G/A | T | G/A | C/G | A | T | G/A | T | C | G/A | G | T | T | C | T | T | C | T | G |
| MLV30-P3 | G | T | C | A | T | G/A | G | A | T | G | T | C/T | G/A | G | T | T | C | T | T | C | T | G |
| MLV30-P4 | G | T | C | A | T | G/A | G | A | T | G/A | T | C/T | G/A | G | T | T | C | T | T | C | T | G |

TABLE 9
Matching and Non-Matching Example Based on SNPTrack Analysis

| | P1S001- | P1S001- | P1S002- | P1S002- | P1S002- | P1S003- | P1S003- | P1S004- | P1S004- | P1S004- | P1S005- | P1S005- | P1S005- | P1S006- |
|---|---|---|---|---|---|---|---|---|---|---|---|---|---|---|
| SNP ID | 245 | 421 | 368 | 533 | 939 | 774 | 805 | 478 | 758 | 866 | 4476 | 4679 | 4904 | 426 |
| Subject | 48521522 | 41855052 | 41855371 | 41854995 | 48521356 | 41855003 | 48521502 | 41855420 | 48521532 | 39180829 | 41855428 | 41854889 | 41855387 | 48526443 |
| MLAC1 | G | C | T | G | T | G | A | A/G | C/G | C | C | A | G/T | A/G |
| PGG1 | G | C/T | 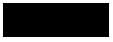 | A/G | T | A/G | A | A/G | C | C | C | A | G | A |
| PGG2 | G | 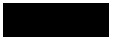 | N/A | A/G | T | A/G | A | A | C | C | C | A | G/T | A |
| PGG3 | G | C/T | C/T | A/G | T | A/G | A/G | A | C | C | C/T | A | G | A |
| PGG4 | G | C/T | C/T | A/G | T | A/G | A | A | C | C | C | A | N/A | A |
| PGG5 | G | C/T | C/T | A/G | T | G | A/G | A | C | C | C | A | G/T | A |
| | P1S006- | P1S007- | P1S007- | P1S008- | P1S008- | P1S008- | P1S009- | P1S009- | P1S010- | P1S010- | P1S011- | P1S011- | P1S011- | P1S012- |
| SNP ID | 810 | 361 | 71 | 115 | 192 | 555 | 111 | 118 | 471 | 524 | 197 | 268 | 75 | 276 |
| Subject | 41855183 | 48521483 | 41855060 | 48521463 | 41854832 | 41855019 | 41855518 | 41855461 | 48521329 | 41855297 | 41855068 | 39180952 | 41855109 | 39180862 |
| MLAC1 | C/G | G | C | A/G | D/I | D | A/G | G | A/C | C/T | C/T | G | C | C |
| PGG1 | G | A/G | C | A/G | D/I | D | A/G | G | A | T | T | G | C | C |
| PGG2 | G | A/G | C | A/G | D/I | D | A/G | G | A/C | T | T | G | C | C |
| PGG3 | G | A/G | C | G | D/I | D | A | G | A | T | T | G | C | C |
| PGG4 | G | A/G | N/A | G | D/I | D | A/G | G | A | T | T | G | C | C |
| PGG5 | G | 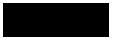 | C | G | I | D | A | G | C | T | C/T | G | C | C |
| | P1S012- | P1S013- | P1S013- | P1S014- | P1S014- | P1S014- | P1S014- | P1S015- | P1S015- | P1S016- | P1S016- | P1S016- | P1S017- | P1S019- |
| SNP ID | 494 | 115 | 515 | 116 | 177 | 477 | 93 | 428 | 97 | 102 | 127 | 193 | 121 | 402 |
| Subject | 41855076 | 47272028 | 39180870 | 48521338 | 39181228 | 47272037 | 41855469 | 47272019 | 41855477 | 41855395 | 41854905 | 41855305 | 41855240 | 41855362 |
| MLAC1 | N/A | G | C | A | C | C/T | A | C/T | C/T | C | A | T | C/T | A |
| PGG1 | C/T | A/G | C/T | A | C | T | A | C | C | C | A | T | C | A/C |
| PGG2 | C/T | G | 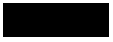 | A | C | T | A | C | C | C | A | T | C | A/C |
| PGG3 | C/T | G | 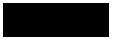 | A | C | T | A/C | C | C | C/G | A | T | C | A/C |
| PGG4 | C/T | A/G | 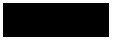 | A | C | T | A/C | C | C | C | A | T | C | A/C |
| PGG5 | T | A/G | 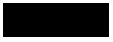 | A | C | T | A/C | N/A | C | C | A | T | C | A |

None of the mothers (PGG1-5) could be the parent of sample (MLAC1)

| | P1S001- | P1S001- | P1S002- | P1S002- | P1S002- | P1S003- | P1S003- | P1S004- | P1S004- | P1S004- | P1S005- | P1S005- | P1S005- | P1S006- |
|---|---|---|---|---|---|---|---|---|---|---|---|---|---|---|
| SNP ID | 245 | 421 | 368 | 533 | 939 | 774 | 805 | 478 | 758 | 866 | 4476 | 4679 | 4904 | 426 |
| Subject | 48521522 | 41855052 | 41855371 | 41854995 | 48521356 | 41855003 | 48521502 | 41855420 | 48521532 | 39180829 | 41855428 | 41854889 | 41855387 | 48526443 |
| MLAC2 | G | C | T | A/G | T | G | A | A/G | C/G | C | C | A/T | G/T | A/G |
| PGG1 | G | C/T | C | A/G | T | A/G | A | A/G | C | C | C | A | G | A |
| PGG2 | G | T | N/A | A/G | T | A/G | A | A | C | C | C | A | G/T | A |
| PGG3 | G | C/T | C/T | A/G | T | A/G | A/G | A | C | C | C/T | A | G | A |
| PGG4 | G | C/T | C/T | A/G | T | A/G | A | A | C | C | C | A | N/A | A |
| PGG5 | G | 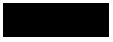 | C/T | A/G | T | G | A/G | A | C | C | C | A | G/T | A |
| | P1S006- | P1S007- | P1S007- | P1S008- | P1S008- | P1S008- | P1S009- | P1S009- | P1S010- | P1S010- | P1S011- | P1S011- | P1S011- | P1S012- |
| SNP ID | 810 | 361 | 71 | 115 | 192 | 555 | 111 | 118 | 471 | 524 | 197 | 268 | 75 | 276 |
| Subject | 41855183 | 48521483 | 41855060 | 48521463 | 41854832 | 41855019 | 41855518 | 41855461 | 48521329 | 41855297 | 41855068 | 39180952 | 41855109 | 39180862 |
| MLAC2 | C/G | G | C | A/G | D/I | D | A/G | G | A/C | C/T | C | G | C | C |
| PGG1 | G | A/G | C | A/G | D/I | D | A/G | G | A | T | 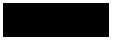 | G | C | C |
| PGG2 | G | A/G | C | A/G | D/I | D | A/G | G | A/C | T | 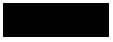 | G | C | C |
| PGG3 | G | A/G | C | G | D/I | D | A | G | A | T | 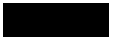 | G | C | C |
| PGG4 | G | A/G | N/A | G | D/I | D | A/G | G | A | T | C/T | G | C | C |
| PGG5 | G | 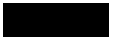 | C | G | I | D | A | G | C | T | C/T | G | C | C |
| | P1S012- | P1S013- | P1S013- | P1S014- | P1S014- | P1S014- | P1S014- | P1S015- | P1S015- | P1S016- | P1S016- | P1S016- | P1S017- | P1S019- |
| SNP ID | 494 | 115 | 515 | 116 | 177 | 477 | 93 | 428 | 97 | 102 | 127 | 193 | 121 | 402 |
| Subject | 41855076 | 47272028 | 39180870 | 48521338 | 39181228 | 47272037 | 41855469 | 47272019 | 41855477 | 41855395 | 41854905 | 41855305 | 41855240 | 41855362 |
| MLAC2 | T | A/G | C/T | A | C | C/T | A | C | C | C | A | T | C | A/C |
| PGG1 | C/T | A/G | C/T | A | C | T | A | C | C | C | A | T | C | A/C |
| PGG2 | C/T | G | T | A | C | T | A | C | C | C | A | T | C | A/C |
| PGG3 | C/T | G | T | A | C | T | A/C | C | C | C/G | A | T | C | A/C |
| PGG4 | C/T | A/G | T | A | C | T | A/C | C | C | C | A | T | C | A/C |
| PGG5 | T | A/G | T | A | C | T | A/C | N/A | C | C | A | T | C | A |

Only PGG4 could be from the parent of MLAC2

Materials and Methods

A. SNP Identification and Development of Markers to Trace or Identify an Individual Animal

DNA Isolation: Blood samples were collected and DNA was extracted from 30 sows from 10 different farms and from approximately 100 boars.

Bioinformatics: Approximately 20 to 40 loci or genes from X chromosome and 10 loci or genes from Y chromosome were analyzed. Pig homologs from EST database were identified. PCR primers to amplify and sequence the identified genes and flanking sequences from pig DNA were designed.

Autosomal Markers: Ligation mediated amplification (LMA) assays from known SNPs in the pig genome were developed. The sequences from flanking regions were expanded to identify additional SNPs. Genotype identification by LMA for about 50 DNA samples was performed to select informative markers.

Mitochondrial Markers: D-loop region from 50 sows was sequenced to identify informative maternal SNPs. Additional mitochondrial DNA as needed was sequenced to increase the information content of the D-loop SNPs.

Developing X and Y SNPs: Sequence tagged sites (STSs) from 5 to 10 individuals were amplified. Sequences were analyzed for SNPs. SNPs with minimum heterozygosity of 10% were identified. A mix of markers with heterozygosities between 10 to 40% was chosen.

Validation of autosomal markers: Allele frequencies in a study population were determined and the power of these markers to trace or identify an individual was estimated. Additional candidate SNPs from databases were identified if necessary.

Mitochondrial LMA SNP assays: LMA assays were developed and the remaining DNA samples were analyzed from sows to establish allele frequencies. Statistical analyses were performed to estimate the mitochondrial markers' power to distinguish between various maternal lineages.

LMA assays and Validation: LMA assays were developed for the X and Y chromosome markers. The allele frequencies based on DNA samples from sows and/or boars were determined.

Marker Validation: SNP assays were performed in a test population blindly to test the mitochondrial markers' ability to identify the maternal lineage.

Statistical analysis: SNP data from sex chromosome markers were combined with the SNP data from mitochondrial and autosomal SNPs. Statistical power of the combined markers was estimated to identify each individual in the study population using the experimental allele frequencies obtained during marker validation. Group markers in sets of 5 to 8 with at least 8 lineage markers were used to genotype all animals. Additional markers may be chosen based on the results obtained with the lineage markers. This process will be repeated until an animal can be identified with certainty. This process will create a SNP marker “tree” with lineage markers at the base, and various X chromosome and autosomal markers defining each branch. It is expected that the actual markers genotyped will differ between animals and will be chosen based on the marker tree generated from the population data collected. The emphasis will be on minimizing the number of markers to be genotyped and as a result of the cost of the test. The marker tree will be validated by blindly genotyping 50 individuals from the population.

B. Statistical Analyses to Evaluate the Power and the Number of Markers Required for Traceability or Identity Studies.

Statistical analyses were developed to evaluate the power and the number of markers required for traceability and identity testings, and to evaluate potential fluctuations in statistical power in actual experimental situations. Statistical power for traceability testing is measured by exclusion probabilities with and without known sires. The following statistical analyses were conducted.

The number of autosomal markers required to achieve a given maximum average exclusion probability for the number of alleles varied from 2 to 10, assuming the use of mtDNA testing with a haplotype (SNPTrack) frequency of 1/10. Known sires and unknown sires were considered.

The effect of mtDNA polymorphisms on the number of autosomal markers required was considered. The results show that the increase in the number of mtDNA haplotypes (SNPTracks) from 10 to 12 results in only a negligible reduction in the number of markers required, e.g., the reduction if 0.2-0.5 markers assuming four alleles with equal allele frequency for each autosome marker, depending on the threshold value of the overall exclusion probability.

The number of autosomal markers required with mixed semen from known sires was considered. The wrong inclusion rate with genotyping mixed semen was compared to that without genotyping mixed semen. This analysis shows that genotyping mixed semen from known sires significantly reduces the wrong inclusion rate and hence significantly reduces the number of markers required to achieve a given exclusion power.

The number of autosomal markers required with different sets of allele frequencies assuming 3 alleles per marker was used to evaluate potential fluctuation in exclusion power due to unequal allele frequency. The results show that the average exclusion power decreases as the differences among allele frequencies increase. The results show that the average exclusion power decreases as the differences among allele frequencies increase.

The results for identity testing show that identity testing is a much easier task than paternity testing. The number of markers sufficient for paternity testing is more than sufficient for identity testing.

a) Algorithms

Algorithms used in the statistical analyses included standard mathematical expressions for exclusion probabilities of autosome markers available in the literature, and five other mathematical expressions derived taking into consideration of the genotyping of mtDNA and mixed semen from known sires, and a likelihood ratio test for identity testing that is found in the literature.

The five other mathematical expressions derived are: 1) Exclusion probability of autosome and mtDNA markers when the sire is known, 2) Exclusion probability of autosome and mtDNA markers when the sire is unknown, 3) Number of markers required to achieve a given exclusion power with equal or unequal allele frequencies and mtDNA markers, 4) Exclusion power of with equal or unequal allele frequencies, mtDNA markers, and mixed semen from known sires, and 5) Number of markers required to achieve a given exclusion power with equal or unequal allele frequencies, mtDNA markers, and mixed semen from known sires.

b) Computer Programs

Six computer programs in SAS computer language (SAS Institute Inc., Cary, N.C.) were developed to evaluate the power and the number of markers required for paternity and identity testings, and to evaluate potential fluctuations in statistical power in real situations. Statistical power for paternity testing is measures by exclusion probabilities with and without known sires.

The six computer programs used are: 1) exclud_max.sas: implements statistical analysis (computer script 1); 2) exclud_max_mt.sas: implements statistical analysis (computer script 2); 3) exclud_err_mix.sas: implements statistical analysis (computer script 3); 4) exclud_a3b.sas: implements statistical analysis (computer script 4); 5) exclud_a4b.sas: implements statistical analysis (computer script 5); and 6) identity.sas: implements statistical analysis (computer script 6)

```
                    1) exclud_max.sas
/* Computer program 1) for statistical analysis 1) */
data exlude;
prohap=1/10;
t100=100000;
mil=1000000;
mil10=10*mil;
mil100=100*mil;
bil=1000*mil;
ext100=1−1/t100;
ex1=1−1/mil;
ex10=1−1/mil10;
ex100=1−1/mil100;
exbil=1−1/bil;
do n=2 to 10 by 1;
    q0=(n−1)*(n**2−3*n+3)/(n**3);
    q1=1−(2*n**3+n**2−5*n+3)/(n**4);
    q1_gm=(n−1)*(n**3−n**2−2*n+3)/(n**4);
    d1=log(1−q1);
    d0=log(1−q0);
    n1_t100=log(1−ext100)/d1;
    n0_t100=log(1−ext100)/d0;
    n1_1=log(1−ex1)/d1;
    n1_10=log(1−ex10)/d1;
    n1_100=log(1−ex100)/d1;
    n1_bil=log(1−exbil)/d1;
    n0_1=log(1−ex1)/d0;
    n0_10=log(1−ex10)/d0;
    n0_100=log(1−ex100)/d0;
    n0_bil=log(1−exbil)/d0;
    n1_t100_mit=log((1−ext100)/prohap)/d1;
    n0_t100_mit=log((1−ext100)/prohap)/d0;
    n1_1_mit=log((1−ext1)/prohap)/d1;
    n1_10_mit=log((1−ex10)/prohap)/d1;
    n1_100_mit=log((1−ex100)/prohap)/d1;
    n1_bil_mit=log((1−exbil)/prohap)/d1;
    n0_1_mit=log((1−ex1)/prohap)/d0;
    n0_10_mit=log((1−ex10)/prohap)/d0;
    n0_100_mit=log((1−ex100)/prohap)/d0;
    n0_bil_mit=log((1−exbil)/prohap)/d0;
    output;
end;
    keep n q0 q1;
    keep n1_1 n1_10 n1_100 n1_bil n0_1 n0_10 n0_100 n0_bil n1_t100 n0_t100
n1_t100_mit
        n0_t100_mit;
    keep n1_1_mit n1_10_mit n1_100_mit n1_bil_mit n0_1_mit n0_10_mit
n0_100_mit n0_bil_mit;
    proc print;
run;
                    exclude.max_mt.sas
/* Computer program 2) for statistical analysis 2) */
data exlude;
t100=100000;
mil=1000000;
mil10=10*mil;
mil100=100*mil;
bil=1000*mil;
ext100=1−1/t100;
ex1=1−1/mil;
ex10=1−1/mil10;
ex100=1−1/mil100;
exbil=1−1/bil;
do nhap = 10 to 20;
  prohap=1/nhap;
  do n=4 to 4 by 1;
    q0=(n−1)*(n**2−3*n+3)/(n**3);
    q1=1−(2*n**3+n**2−5*n+3)/(n**4);
    q1_gm=(n−1)*(n**3−n**2−2*n+3)/(n**4);
    d1=log(1−q1);
    d0=log(1−q0);
    n1_t100=log(1−ext100)/d1;
    n0_t100=log(1−ext100)/d0;
    n1_1=log(1−ex1)/d1;
    n1_10=log(1−ex10)/d1;
    n1_100=log(1−ex100)/d1;
    n1_bil=log(1−exbil)/d1;
    n0_1=log(1−ex1)/d0;
    n0_10=log(1−ex10)/d0;
    n0_100=log(1−ex100)/d0;
    n0_bil=log(1−exbil)/d0;
    n1_t100_mit=log((1−ext100)/prohap)/d1;
    n0_t100_mit=log((1−ext100)/prohap)/d0;
    n1_1_mit=log((1−ex1)/prohap)/d1;
    n1_10_mit=log((1−ex10)/prohap)/d1;
    n1_100_mit=log((1−ex100)/prohap)/d1;
    n1_bil_mit=log((1−exbil)/prohap)/d1;
    n0_1_mit=log((1−ex1)/prohap)/d0;
    n0_10_mit=log((1−ex10)/prohap)/d0;
    n0_100_mit=log((1−ex100)/prohap)/d0;
    n0_bil_mit=log((1−exbil)/prohap)/d0;
    output;
  end;
end;
    keep prohap nhap;
* keep n1_1 n1_10 n1_100 n1_bil n0_1 n0_10 n0_100 n0_bil n1_t100 n0_t100;
    keep n1_t100_mit n1_1_mit n1_10_mit n1_100_mit n1_bil_mit
        n0_t100_mit n0_1_mit n0_10_mit n0_100_mit n0_bil_mit;
    proc print;
run;
                    exclude_err_mix.sas
/* Computer program 3) for statistical analysis 3) */
data exclude;
mit_prob=0.10;
do m=1 to 36 by 1;
  do n=4 to 4 by 1;
    q0=(n−1)*(n**2−3*n+3)/(n**3);
    q1=1−(2*n**3+n**2−5*n+3)/(n**4);
    wrong_inc0=log10((1−q0)**m);
    wrong_inc1=log10((1−q1)**m);
    q0_m=1−(1−q0)**m;
    q1_m=1−(1−q1)**m;
    wrong_inc0_mit=(1−q0)**m*mit_prob;
    wrong_inc_mix=1−qmix_m_mit;
    q0_m_mit=1−(1−q0)**m*mit_prob;
    q1_m_mit=1−(1−q1)**m*mit_prob;
    qmix_m_mit=q0_m*q1_m_mit + (1−q0_m)*q0_m_mit;
    wrong_inc0_mit=(1−q0)**m*mit_prob;
    wrong_inc_mix=1−qmix_m_mit;
    err_ratio = wrong_inc0_mit/wrong_inc_mix;
    output;
  end;
end;
data _null_;
set exclude;
* user needs to modify the following statement for file location;
file “file location”;
put m 4. n 4. wrong_inc0_mit 15.12 wrong_inc_mix 15.12 q0_m_mit 15.12
q1_m_mit 15.12
    qmix_m_mit 15.12 err_ratio 10.2;
cards;
run;
                    exclude_a3b.sas
/* Computer program 4) for statistical analysis 4) */
data exlude;
  input p1 p2 p3;
p_mt_hap=0.10;
mit_ex=1−0.1;
l1=1/prohap;
mil=1000000;
mil10=10*mil;
mil100=100*mil;
bil=1000*mil;
ex1m=log10(1/mil);
ex10m=log10(1/mil10);
ex100m=log10(1/mil100);
exbil=log10(1/bil);
do;
    p1m=1−p1;
    p2m=1−p2;
    p3m=1−p3;
    p1ms=p1m**2;
    p2ms=p2m**2;
    p3ms=p3m**2;
    p12=p1*p2;
    p13=p1*p3;
    p23=p2*p3;
    p12m=1−p1−p2;
    p13m=1−p1−p3;
    p23m=1−p2−p3;
    p24m=1−p2−p4;
    p34m=1−p3−p4;
    p12ms=p12m**2;
    p13ms=p13m**2;
    p23ms=p23m**2;
    p1s=p1*p1;
    p2s=p2*p2;
    p3s=p3*p3;
    p4s=p4*p4;
    u12=4−3*(p1+p2);
    u13=4−3*(p1+p3);
    u23=4−3*(p2+p3);
    v12=p12*p12ms;
    v13=p13*p13ms;
    v23=p23*p23ms;
    q0=p1s*p1ms+p2s*p2ms+p3s*p3ms
        +2*(v12 + v13 + v23);
    q1=p1*p1ms+p2*p2ms+p3*p3ms
        −(p1s*p2s*u12+p1s*p3s*u13+p2s*p3s*u23);
    d1=log10(1−q1);
    d0=log10(1−q0);
    n1_1=int(ex1m/d1+0.5);
    n1_10=int(ex10m/d1+0.5);
    n1_100=int(ex100m/d1+0.5);
    n1_bil=int(exbil/d1+0.5);
    n0_1=int(ex1m/d0+0.5);
    n0_10=int(ex10m/d0+0.5);
    n0_100=int(ex100m/d0+0.5);
    n0_bil=int(exbil/d0+0.5);
    n1_1_mt=int((ex1m−log10(p_mt_hap))/d1+0.5);
    n1_10_mt=int((ex10m−log10(p_mt_hap))/d1+0.5);
    n1_100_mt=int((ex100m−log10(p_mt_hap))/d1+0.5);
    n1_bil_mt=int((exbil−log10(p_mt_hap))/d1+0.5);
    n0_1_mt=int((ex1m−log10(p_mt_hap))/d0+0.5);
    n0_10_mt=int((ex10m−log10(p_mt_hap))/d0+0.5);
    n0_100_mt=int((ex100m−log10(p_mt_hap))/d0+0.5);
    n0_bil_mt=int((exbil−log10(p_mt_hap))/d0+0.5);
    output;
    keep ex1m ex10m ex100m exbil n1_1 n1_10 n1_100 n1_bil n0_1 n0_10 n0_100
n0_bil
        n1_1_mt n1_10_mt n1_100_mt n1_bil_mt n0_1_mt n0_10_mt n0_100_mt
n0_bil_mt;
end;
cards;
0.33 0.33 0.34
0.4 0.3 0.3
0.5 0.3 0.2
0.6 0.3 0.1
0.7 0.2 0.1
  ;
title ‘Number of loci required to achive a given exclusion power’;
proc print;
run;
                    exclude_a4b.sas
/* Computer program 5) for statistical analysis 5) */
data exlude;
    input p1 p2 p3 p4;
p_mt_hap=0.10;
mit_ex=1−0.1;
l1=1/prohap;
mil=1000000;
mil10=10*mil;
mil100=100*mil;
bil=1000*mil;
ex1m=log10(1/mil);
ex10m=log10(1/mil10);
ex100m=log10(1/mil100);
exbil=log10(1/bil);
do;
    p1m=1−p1;
    p2m=1−p2;
    p3m=1−p3;
    p4m=1−p4;
    p1ms=p1m**2;
    p2ms=p2m**2;
    p3ms=p3m**2;
    p4ms=p4m**2;
    p12=p1*p2;
    p13=p1*p3;
    p14=p1*p4;
    p23=p2*p3;
    p24=p2*p4;
    p34=p3*p4;
    p12m=1−p1−p2;
    p13m=1−p1−p3;
    p14m=1−p1−p4;
    p23m=1−p2−p3;
    p24m=1−p2−p4;
    p34m=1−p3−p4;
    p12ms=p12m**2;
    p13ms=p13m**2;
    p14ms=p14m**2;
    p23ms=p23m**2;
    p24ms=p24m**2;
    p34ms=p34m**2;
    p1s=p1*p1;
    p2s=p2*p2;
    p3s=p3*p3;
    p4s=p4*p4;
    u12=4−3*(p1+p2);
    u13=4−3*(p1+p3);
    u14=4−3*(p1+p4);
    u23=4−3*(p2+p3);
    u24=4−3*(p2+p4);
    u34=4−3*(p3+p4);
    v12=p12*p12ms;
    v13=p13*p13ms;
    v14=p14*p14ms;
    v23=p23*p23ms;
    v24=p24*p24ms;
    v34=p34*p34ms;
    q0=p1s*p1ms+p2s*p2ms+p3s*p3ms+p4s*p4ms
        +2*(v12 + v13 + v14 + v23 + v24 +v34);
    q1=p1*p1ms+p2*p2ms+p3*p3ms+p4*p4ms
−(p1s*p2s*u12+p1s*p3s*u13+p1s*p4s*u14+p2s*p3s*u23+p2s*p4s*u24+p3s*p4s*u34);
    d1=log10(1−q1);
    d0=log10(1−q0);
    n1_1=int(ex1m/d1+0.5);
    n1_10=int(ex10m/d1+0.5);
    n1_100=int(ex100m/d1+0.5);
    n1_bil=int(exbil/d1+0.5);
    n0_1=int(ex1m/d0+0.5);
    n0_10=int(ex10m/d0+0.5);
    n0_100=int(ex100m/d0+0.5);
    n0_bil=int(exbil/d0+0.5);
    n1_1_mt=int((ex1m−log10(p_mt_hap))/d1+0.5);
    n1_10_mt=int((ex10m−log10(p_mt_hap))/d1+0.5);
    n1_100_mt=int((ex100m−log10(p_mt_hap))/d1+0.5);
    n1_bil_mt=int((exbil−log10(p_mt_hap))/d1+0.5);
    n0_1_mt=int((ex1m−log10(p_mt_hap))/d0+0.5);
    n0_10_mt=int((ex10m−log10(p_mt_hap))/d0+0.5);
    n0_100_mt=int((ex100m−log10(p_mt_hap))/d0+0.5);
    n0_bil_mt=int((exbil−log10(p_mt_hap))/d0+0.5);
    output;
    keep ex1m ex10m ex100m exbil n1_1 n1_10 n1_100 n1_bil n0_1 n0_10 n0_100
n0_bil
        n1_1_mt n1_10_mt n1_100_mt n1_bil_mt n0_1_mt n0_10_mt n0_100_mt
n0_bil_mt;
end;
cards;
0.25 0.25 0.25 0.25
0.4 0.3 0.2 0.1
0.5 0.2 0.2 0.1
0.6 0.2 0.1 0.1
0.7 0.1 0.1 0.1
    ;
title ‘Number of loci required to achive a given exclusion power’;
proc print;
run;
6)identity.sas
/* Computer program 6) for statistical analysis 6) */
data exlude;
prohap=0.10;
mit_ex=1−0.1;
l1=1/prohap;
mil=1000000;
mil10=10*mil;
mil100=100*mil;
bil=1000*mil;
p_mil=1/mil;
p_mil10=1/mil10;
p_mil100=1/mil100;
p_bil=1/bil;
do n=2 to 10 by 1;
    n2=n**2;
    pii=1/n2;
    pij=2/n2;
    kii_mil = −log10(p_mil)/(2*log10(n));
    kii_mil10 = −log10(p_mil10)/(2*log10(n));
    kii_mil100 = −log10(p_mil100)/(2*log10(n));
    kii_bil = −log10(p_bil)/(2*log10(n));
    kij_mil = log10(p_mil)/(log10(2)−2*log10(n));
    kij_mil10 = log10(p_mil10)/(log10(2)−2*log10(n));
    kij_mil100 = log10(p_mil100)/(log10(2)−2*log10(n));
    kij_bil = log10(p_bil)/(log10(2)−2*log10(n));
    output;
    keep kii_mil kii_mil10 kii_mil100 kii_bil kij_mil kij_mil10 kij_mil100
kij_bil;
end;
proc print;
run;
```

C. Exclusion Probabilities with Autosome and mtDNA Markers

Exclusion Probabilities with Autosome and mtDNA Markers

The overall exclusion probability with or without known sire for a marker set with autosome and mtDNA markers was derived based on previously available exclusion probabilities for autosome markers.

Let
Q₁ₖ=exclusion probability of locus k with known genotypes for the sire and offspring,
Q₀ₖ=exclusion probability of locus k with known genotype for the offspring only.

Then,

$${Q}_{1 k} = {\sum }_{i = 1}^{n} {{p}_{i} ( 1 - {p}_{i} )}^{2} - {\sum }_{i}^{n - 1} {\sum }_{j = 1 + 1}^{n} {p}_{i}^{2} {p}_{j}^{2} ( 4 - 3 {p}_{i} - 3 {p}_{j} ) \qquad ( 1 )$$

(Jamieson, 1965, 1995; Garber and Morris, 1983; Weir, 1996)

$${Q}_{0 k} = {\sum }_{i = 1}^{n} {{p}_{i} ( 1 - {p}_{i} )}^{2} - 2 {\sum }_{i}^{n - 1} {\sum }_{j = i + 1}^{n} {p}_{i} {{p}_{j} ( 1 - {p}_{i} - {p}_{j} )}^{2} \qquad ( 2 )$$

(Garber and Morris, 1983).

Assuming equal allele frequency, equations (1-2) reduces to

$$\begin{matrix}{Q}_{1 k} = \frac{( n - 1 ) ( {n}^{3} - {n}^{2} - 2 n + 3 )}{{n}^{4}} & ( 3 ) \\ {Q}_{0 k} = \frac{( n - 1 ) ( {n}^{2} - 3 n + 3 )}{{n}^{3}} & ( 4 )\end{matrix}$$

The overall exclusion probability with or without known sire for a marker set with autosomal and mtDNA markers was derived based on standard exclusion probabilities for autosomal markers.

D. Analysis of Paternity Testing

Equations (1-2) were implemented in computer programs 4 and 5, and equations (3-4) were implemented in computer programs 1, 2, and 3. Note that equations 1 and 2 are general and covers the cases of equations 3 and 4. These two sets of equations were implemented to provide a mutual check for the correctness of the programming code.

Let p=the equal haplotype frequency assumed for each mtDNA haplotype

Q_{i,mit}=probability that a random individual is excluded as the parent by at least one autosome locus or the mtDNA haplotype when m autosome markers and one mtDNA haplotype are genotyped

Then,

$${Q}_{i , mit} = 1 - p {\prod }_{k = 1}^{m} ( 1 - {Q}_{1 k} ) , i = 1 , 0 \qquad ( 5 )$$

Equation (5) is used in computer programs 1 through 5.

E. Exclusion Probabilities with Added Genotyping for Mixed Semen

A common practice in commercial swine production is the use of mixed semen from a number of sires. If the mixed semen on a dam is genotyped, the exclusion is expected to improve, but non of the above mathematical expression provide the correct estimate of exclusion probability with added genotyping for mixed semen.

Let Q₀=probability that a random individual is excluded as the parent by at least one autosome locus when no known parent is present. Qₘᵢₓ=probability that a random individual is excluded as the parent by at least one autosome locus or the mtDNA haplotype when the sires that were sources of the mixed semen are genotyped for the m autosome markers (potential dam and sow genotyped for autosome and one mtDNA haplotype).

The probability of Q₀ can be used as the probability that the true sire of the disputed offspring can be determined. If any random individual is included as a potential true sire, two of the sires contributing to the mixed semen will be included. In this case, identifying the true sire is considered failed. The mathematical expressions for Q₀ and Qₘᵢₓ are

$$\begin{matrix}{Q}_{0} = 1 - {\prod }_{k = 1}^{m} ( 1 - {Q}_{0 k} ) & ( 6 ) \\ {Q}_{mix} = {Q}_{0} {Q}_{1 , mit} + ( 1 - {Q}_{0} ) {Q}_{0 , mit} & ( 7 )\end{matrix}$$

The equations 6-7 are implemented by computer program 3.

F. Number of Autosome Markers Required

The number of autosome markers required is derived assuming all autosome markers have the same allele frequencies. Under this assumption, the number of markers required is
n=log[(1−Q)/p]/log(1−Qᵢ), i=0, 1 (8)
where Q=the required overall exclusion probability, p=mtDNA haplotype frequency of sow being tested, and Qᵢ is given by equation (1) or (2). Equation (8) is implemented in computer programs 1 through 5.

The analysis of paternity testing is implemented by a computer program. The program will conduct an exhaustive allele matching analysis between the offspring, all potential parents, and any known parent (in some cases the sire may be known). The final results of the analysis will identify the true parent, and a likelihood ratio test showing the reliability of test results.

G. Estimation of Allele Frequencies

Estimates of allele frequencies affect the reliability statement about the testing results but do not affect the actual testing process. Genotyping the entire population gives the most reliable estimates of allele frequencies but is the most costly and time consuming method. A two-step strategy for estimating allele frequencies is proposed. The first step is to genotype sires and dams and then to predict the population allele frequencies based on the alleles observed in the breeding population (sires and dams) and the relative contribution of each sire and dam to the next generation. This first step is possible because DNA samples from sires and dams are available given that DNA bank for sires and dams is in place and that breeding records are available. The second step is to update the estimates of allele frequencies as more genotyping results become available.

H. Sequences and SNP Data of the Markers Used for SNPTrack Analysis in Swine

| | |
|---|---|
| 1 | ctgacagtta aagactgccc aacagtgaag tgaactgcct aaaaaacagt gagttttcta |
| 61 | ttttttatgt gttcaaatga aggaaaaata aatctgtcca ttatggggat aaggRgatac |
| 121 | cagtgttcaa ggggagttaa aacaaaaaga tttctaatgt accttcaaat tcttaagatt |
| 181 | ccatgaaactg(TG)aatttatataggaataa aaaagtgaaa ctattctctt gatatgcaaa |
| 241 | gatgaggaaa aaagatttac atgataaaay ttcaaaataa atcgtttgcc attttaagct |
| 301 | gtattgttcg agctcaagaa ccttctttaa caatatttcc atctttctaa tttataatta |
| 361 | tccaataaaa tatacaatta cctaccactc caaattttaa agtaaatatg tttgagatca |
| 421 | atgtgcagat gaaaggtytt atttgtataa gaggaaagat agtgctatgt aaatacccct |
| 481 | ttcccatcaa gtatattcct atgcacttcc ataaaggcaa ttcagtgtgt attttcacag |
| 541 | gatccttggttattg(G)ttcattttaggtctactgacgaagc agaccttcag aaaaatattt |
| 601 | accctgaatt agagsatcaa gatggtggat gagtaagaca tggagcttac cttcttcc |

The sequence in ( ) is an in/del. In/del at position 556 (G insertion.

Three SNPs:

| Position 115 | 192/193 | 556 indel | Estimated Frequency |
|---|---|---|---|
| G | A | G | 18% |
| G | A | T | 34% |
| G | T | T | 15% |
| A | T | T | 33% |

Two SNPs:

| Position 115 | 192/193 | Estimated Frequency |
|---|---|---|
| G | A | 52% |
| G | T | 15% |
| A | T | 33% |

ACY-STS7:

R = A/G; Y = C/T; K = G/T; M = A/C; S = G/C; W = A/T; V = A/C/G
121 cagcaggatt tttgctgagt ttttttggaa accccctcag gaccaacccc caccccccca
181 aaaagtatta agcaccaaag ttaatagaag agattcacag caaacaaggc agaaccagag
241 accaSgggta caggggagac aacaaaccag gaccagggct caatctttct gctcccaccc
301 tacagcctca gtcttctatg ctaatcctga gaaatcccta gcatgggaag ggacactgca
361 aagcactgta ctgacctagc actggatcag atcaaggtca tatggctggt caatgagcaa
421 Ygtaaaactt acaaggtact gggtacacac agccaagggc atcccttccc ttgaaaagct
481 cttaagccag ggagataaga caacctgccc tcagaaggca ggttacactt gcctaggggg
541 ttataccctg gcccagtaaa ggtcaggcaa agctttacta tgggcctggc agagcatgaa
601 gtccaggcaa aagctggcta ggcagagaaa aattgtgggc tttggtaggc caagtaagat
661 gaaggacact taataataat agcactcagg caggggctga agccagcaaa ggctacatga
721 aagatcctga ctgtgcaaat ggagccaaag agacacactt ctgtgtgatt ccgagcacaa
781 actcaccccc ttaagagatt catatcgttg tgatcggggt ttcttgatgc cgtttctgtg
841 ccattttcgg gctgtggaga agtaaagcat taggtcaaca aaatagattt cccccaataa
901 gaccactacc ccagc

There are 3 expected alleles when combining the information from both positions:

| | | |
|---|---|---|
| Position | 245 | 421 |

| | | |
|---|---|---|
| G | C | 52% |
| C | C | 18% |
| G | T | 30% |

EG-STS7

| | |
|---|---|
| 1 | ggtccctgyg ggtycacgtg ggttggtgtc tacccgtctt cacaagctgg tactgatttg |
| 61 | gtacgttctc tgccttatgg gttctgtgct actgatctat atgtcttcct ggttcattca |
| 121 | tgcctgaggt gctttagatt agttggcatt gtttacgggt aataccaaca gttaacactt |
| 181 | atacccagaa ctcaccacgt cccggggcac agctgcactg cgtgtatata aattccttcg |
| 241 | cccctaaggg gaggtacttc tatgaaccct gctttactaa cgaccaaatg gagcccagac |
| 301 | gtcaggtcgc tttacRgcac atagtgact tgatcccagg gtggctctgc tgccacttgc |
| 361 | cgatctgtct tggttgacat gggctgggct gtcccttaga gtcagacctt tccccagggc |
| 421 | aaaggccact acaagtcagg ggcctaagca gcaaagctga ccatggcctc gccagctcac |
| 481 | cagccttccc tggctccctg ttgcctgcag ggtgtggtcc tgctcrggcg cttcctgttt |
| 541 | tcctctccaa gacttcttcc ctcactctgc ccaaaacatc cttcttcccc ttctgcatcc |
| 601 | caccagctcc aacgtaggct tcaagatgYc tcctccagga agtcctccca gctgtgctcc |
| 661 | tctccacact ccccgctcag ttgatgtctc crccgcacrc acgtccctca tccagcactt |
| 721 | cctgtgacag tgcttctccc cctgcatctc cccccgtgag cctcagactg gccRttcccg |
| 781 | gaagagcrgt gaYrtggatg agtgRcccag agttagcRac ctagagctga ggggccatct |
| 841 | cccagtcctg tggcccttac tccccagccg caccccctYg gRcagggagc acagggaggg |
| 901 | ctgctggtgt gttctagcca tggcccgatg accyttgcYg cctccccatg ctgtgttcct |
| 961 | gggctgggga agggtctcca cagggaaggg agaggttgac aggagagccc cctgccccta |
| 1021 | Ytgccctggg gacac |

Positions to score

Three SNPs:

| Position 774* | 805 | 817 | Estimated frequency |
|---|---|---|---|
| A | A | G | 17% |
| G | A | A | 15% |
| G | A | G | 22% |
| G | G | G | 46% |

Same as 773 on previous sheets

Alternate Two SNPs:

| Position 793* | 805 | Estimated frequency |
|---|---|---|
| T | A | 17% |
| C | A | 37% |
| C | G | 46% |

Same as 792 on previous sheets

Alternate SNPS

| position 879 | |
|---|---|
| C | 73% |
| T | 27% |
| Position 882 | |
| A | 83% |
| G | 17% |

RYRA-STS6

| | |
|---|---|
| 1 | gaccaagagc tgcagcaccg tgtggagtcc ctggcagcct ttgcagaacg ctacgtggac |
| 61 | aagctccagg ccaaccagag ggaccgctat ggcatcctca tgaaggcctt caccatgacc |
| 121 | gctgccgaga ctgcccgacg tactcgcgag ttccgctccc caccccagga gcaggtcctc |
| 181 | taacccccaa actcagctgg ccttactgtc tcaacctcag cctctcccct tactctgatc |
| 241 | actgatggca ctcaacctct aaacctgggc ttgacctctg atcctgtggg tatacttctc |
| 301 | tcttgctccc ctacctctct ctgacccaga tttcrgagtc agcccagact gaccctaagt |
| 361 | cctttcaaac ctttgatctc ccagatattc ctcagtaact cMtgactSca gacaggggct |
| 421 | cagttggatc ttagatcctt gacctcagag ttcctgctcc ggggtctctg accctcattc |
| 481 | taacctttga ccttccctag atcaacatgc tattgcactt caaagatggc gaggatgagg |
| 541 | aagattgtcc tcttcctgat gagatcMggc aggatttgct ggaattccat caagacctgt |
| 601 | tgactcactg tggtaagaga ggatatcagg gaatcctctt ccccagtttt ttctcgagac |
| 661 | ctctctgaaa gtttccctaa gatttcctga tcttggagtt cccgtcgtgg cgcagtggtt |
| 721 | macgaatccg actaggaacc atgaggttgc gggttcggtc cctgcccttg ctcagtgggt |
| 781 | tamygatccg gcgttgccgt gagctgtggt gtaaggttgc aaacccagct caa |

Positions to score

Two SNPs:

| Position | 408 | 567 | Estimated frequency |
|---|---|---|---|
| | C | A | 35% |
| | C | C | 43% |
| | G | C | 22% |

Three SNPs:

| 402 | 408 | 567 | Estimated frequency |
|---|---|---|---|
| A | G | C | 22% |
| A | C | C | 22% |
| A | C | A | 36% |
| C | C | C | 22% |

PBE59

| | |
|---|---|
| 1 | ttctaaagtt cagcatactt cactagtgat acatgtctta Ytgatacttc cttaagagtt |
| 61 | atgtgcttac ctgcctaggc cctccctcca ctagatggct cagccctggg gatcaggYgt |
| 121 | tatctcctta gctgcatgaa gctrgagtMg tgtgttgtgc acaccagaat ccacctgcgt |
| 181 | tcaacaccta gctctggagc tcctgctatg gaccaggcat tgtttctggt gccrctgatg |
| 241 | cagYggagag caaatcagat cccacaaacc catgaYgctc rcatgtgcat Racggaggaa |
| 301 | aaaatatcaa ggaagaagca aatgaaatga gaatacagga ctcctgacct agtccctact |
| 361 | arccagaagt tgtctccaaa grttttcatt ttctatgccy gtggatgatg gtcagaaaga |
| 421 | agatctgcct gtaatcatgt tctcgaaggg atccaagacc tymagcaacc agaaaggaac |
| 481 | tacttcacag taaYactgtt ccaaaccaac agtaagatgc ccgttccctc acttcgcttc |
| 541 | catcttcttt aacrtcaagc agtccttgga gctagctact tcttagtcgt aagaactcga |
| 601 | acgtacataa cgtatttgca gtttccaaag cacatttcc |

Two SNPs:

| Position | 276 | 494 | Estimated frequency |
|---|---|---|---|
| | C | T | 20% |
| | C | C | 30% |
| | T | C | 50% |

PBE43

| | |
|---|---|
| 1 | cctgaagaat tgatacatta atgtgctatt tcatagcgtg ataagaatgt gcctttgcca |
| 61 | tctcctttga aatgcaaaca tctttattct ttaggggaga ctttgtttac tttgattcaa |
| 121 | cagtgmaaaa aattgggaat tagaaacctt tctgtagttt cccagaagct ggctcttagc |
| 181 | acagattttt ggtttctctc actggagttg acttgcatcg aaagttggga gcaaccctaa |
| 241 | aaggtatgac ctaaaatcaa gctggtggca gagtagggga gtctgtacat agcgccgggg |
| 301 | gttctgagcgtgc(tgtagtg tgtgtastgt akttctsmgt) gtggggaatc ctcaagacag |
| 361 | ggagtccyag gggcctgtag gtattcctct tcctgaaatc atggaatggg tgagccggaa |
| 421 | ggagaacctt ctattctttg ctggccttat ttctttttck ttccctctca Maagttacag |
| 481 | aggtggcttc acagatccag gtcctgctgg agatcttcgg ctgYccctga gaakccagga |
| 541 | agatttttac taaaaaytta ctyttccatc tcctctgcta saaaggccgc cactgtcgct |
| 601 | ttggcctcca cagaggccac aacaccctca gctccagagt cttcactgaa tgtacctgct |
| 661 | ttcacatgaa ca |

Three SNPS:

| Position | 314 | 471 | 524 | Estimated frequency |
|---|---|---|---|---|
| | T | C | C | 21% |
| | G | A | T | 25% |
| | G | C | T | 40% |
| | G | C | C | 14% |

GALT

| | |
|---|---|
| 1 | ggatccttaa cccactgagt gaggccaggg atcgaatttg cattctcgta gatactggtc |
| 61 | agatttgttt ctgctgggcc accatgggaa ctccctggtt ttgtctatat atattttttt |
| 121 | ttttttttgt cttttttgcc atttcttggg ccgctcctgc ggcatatgga ggttcccagg |
| 181 | ctaggggtcg aatcggagct gtagccacca gcctacgcca gagccacagc aacgtgggac |
| 241 | ccgagccgag tctgcaacct ataccacagc tcacggcaac gccagatccc ttaacccact |
| 301 | gagcaaggcc agggaccgaa cccgcaacct catggttctt agtcggattc gttaaccact |
| 361 | gcgccacgac gggaactccc ggttttgtct atttttgaac gttaaataaa tgcaagcatc |
| 421 | cagggctgct ttgactcagt accatgtgtg agatttaccc tgttgatgtc agcagctRtg |
| 481 | gctggttcct tctcacggat gtgtgtgacc ctcacctgga ccacacctga tctggctgat |
| 541 | gatgggcctt ggggtttttc cagcttttgg tcccakgtca cgtctctgtt tgaacttaaa |
| 601 | tgcacttgct ttcaggtatt aatctggggc ggaatgactg gaacatgagg tgtggttggt |
| 661 | tcagctttag tagatgccag cagggaggat ttcagtagtt tattaagcag atcttgaaga |
| 721 | ctgtggtcaa ctagctcatg ccccagagga gggggcgStg aatttcttcc ccagaacagg |
| 781 | agtgagaagc taaattaggc atccatccgc tggaagytga gggggcagtt cttggctcct |
| 841 | ttctgtcagg tttcggcccc ttctcSttag tctggggttt ctaggctcta ctcccaggaa |
| 901 | gwgtctgggg ccacttggga agaatgggtg ggggggctyt gagcccctac ttacttcatt |
| 961 | tccctccttc agccaaarcc ycctgtgtcc tctgttttac atagtggggt tctgagaatg |
| 1021 | acttywtttt tttttttttt tttttwaaag ctttagctrt kgcgacattt acaaatccmc |
| 1081 | tgctgtgagg tctcttccag ctaggaaatt ctattttggr ascagraggt gggtgtgggr |
| 1141 | agggttaagc attattcagc caaagagttg ggttgggcct cagtgacctt ttgaagttct |
| 1201 | tatagcttgg cttgccatgc aggagatctc agaacattct ataaaaatag tgttcaaaca |
| 1261 | gaacaacttc tgaagcctaa aggatgcgaa caagaggctc ggaaggtagc atttcaacgg |
| 1321 | gagttttgag gatgctctcc tttagccacc cctctccatt ttctgccccc ttctttttaa |
| 1381 | attctccatt ggctgtccct gctagttgtc atttggggtg gtttgggttc agaatggttc |
| 1441 | tcattttcgc cgaggagtgg gtgatgtggg cggcctgtgt gtctctccca agggtggtgg |
| 1501 | ctgtccctcc tccaccacca ggcctagttt ggacctgtag tttcgcttag tgaaggaggc |
| 1561 | cgggccgatc ctgggccgga gagagacgtm tcatgccwtg gcatgcagct ctgagtcaac |
| 1621 | aggcctgata aacagcccac ttcccagggc gagcaaggag gaacaaggcc cctggctgct |
| 1681 | gtgggatccg tctgcgctcc tcttcgtgaa accgctgttt attcttttga caggagttgg |
| 1741 | aacgcagcac cttcccttcc tcccagccct gcctccttct gcagagcaga gctcactaga |
| 1801 | acttgtttcg ccttttactc tggggggaga gaagcagagg atgag |

Two SNPs:

| Position | 478 | 758 | Estimated frequency |
|---|---|---|---|
| | A | C | 47% |
| | G | C | 12% |
| | G | G | 41% |

Three SNPs:

| Position | 478 | 758 | 866 | Estimated frequency |
|---|---|---|---|---|
| | A | C | C | 47% |
| | G | C | C | 12% |
| | G | G | C | 17% |
| | G | G | G | 24% |

VAN-STS1

| | |
|---|---|
| 1 | gaatttgtct cagtarttaa tgactttaaa gctrcaaaag aattaagaaa gaaatagcta |
| 61 | ttaccaggcc aggaagataa aaaccttatc agagacaata tatcagttgt gaagaatcct |
| 121 | ggttctgttt taaagataaa agttagmctt amggcacatt gcttaaatkg ttttacagct |
| 181 | caaccagccc atcaagtact caaccaccag gccaagtgga acctaagaaa ggatgatgcc |
| 241 | agccttggct gacccttgta actctaatca gctggacctt tgccccagtt ctatgctgaa |
| 301 | ttcttcyttg ctcaagccct ttcatgagta tgaatgtacc cttaryttaa aacttcccca |
| 361 | gttttgctgt ttgagagaca ttgttttggg aactatccct gatactcttc ttactgttaa |
| 421 | gtaaaacaaa tccttcctac tcctgctctt tggcttgact gtgtcttttg gctcaaaacc |
| 481 | caaygagagg tgaacccagt tttggggtga cattagggat caggtggatc aactcaggca |
| 541 | tmgtaggaaa agctactggt gttccaccag ttccagctta agctcatgga tggcattctc |
| 601 | yagcaagaat ygcaattgtc cacctaaaga tgttctctca gcttctgtgg gccaagtagg |
| 661 | ccagaaatgc cagattrgga gttcctgtgg tagctcagca gattaagaac cctactcagt |
| 721 | ktctgygagg atgcaggttt gttcctgaag attargttct tgtctagttg cagaaggaat |
| 781 | tcagaaatga gatggaaatt gagaaagaaa agtgagggtt tatttaagtg agaagtacac |
| 841 | ctcttaag(gagaggtgggc)agagaggtggg cagagaggtg agcagctgYc ctgtgttttt |
| 901 | tgggtgcact agttagaagg ggtgtccaat tgtatagatg ggatagtcaY tgagaaagtg |
| 961 | gggtttaggg gtcatattcc ttaattttca ycccagctcc accttcccRa agggaggagg |
| 1021 | gatttttgtc cttagttggt taattggaag tgtcatggca tccaYrtata atgggtactt |
| 1081 | cttatctgca tagctaattg tattakaatt ttattataag gagggcataa tgagcaacag |
| 1141 | kgttccattc agacactgga gattcgkgcc ctcttctacc tttctttgtc tgcagcctgg |
| 1201 | gcacttatca ccccaaaaat gtgtgrtttc ctatcagtct ggtgkttccr gcttttcttt |

The sequence (gagaggtgggc) in bold is an In/Del.

Positions to score:
Two SNPS:

| position | 849 | 889 | Estimated frequency |
|---|---|---|---|
| | G | C | 54% |
| | A | C | 15% |
| | A | T | 31% |

Three SNPs:

| position | 950 | 1009 | 1065 | Estimated frequency |
|---|---|---|---|---|
| | C | G | C | 60% |
| | C | G | T | 8% |
| | T | G | T | 13% |
| | C | A | C | 19% |

IKBA

| | |
|---|---|
| 4321 | gctactcccc gtaccagctc acctggggcc gcccaagcac tcggatacag cagcagctgg |
| 4381 | gccagctgac cctagaaaac ctccagatgc ttccagagag cgaggatgag gagagctatg |
| 4441 | acacggagtc agagttcaca gaggatgagg tgagtYccaa tgaccttgtt cacgggtctg |
| 4501 | caaaaagcaa tgctctcgga cccctagagc tcctcctttt cctgagggtc tcaacataat |
| 4561 | gaggatctca aattagggag cataagcagt gtcctaagag taggtttagg gggaggatta |
| 4621 | tggtytgggg ttttcttttg cttttttgct ctttttgaag gagaggatcc ttaaaggaWa |
| 4681 | acttcagccc aggaagttaa ttcagattcg ggttagaggg aacggagtcc aagaatactt |
| 4741 | gcgttatttc cagtagcagc ccttgccatc accccagcac ctttggcaaa gttctggaag |
| 4801 | tttaacatgc ctttctttcc ccttttagct gccctatgac gactgcgtgc ttggaggcca |
| 4861 | gcgcctgacg ttatgagctt tggaaagtgt ctaaaagacc atgKacttgt acatttgtac |
| 4921 | aaaatcaaga gttttatttt tctaaaaaaa aagaaaaaaa gaaaaaaaaa gaaaaaaggg |
| 4981 | tatacttata accacaccgc acactgcctg gcctgaaaca ttttgctctg gtggattagc |
| 5041 | cccgattttg ttattcttgt gaactttgga aaggcgccaa ggaggatcat cggaatgcag |
| 5101 | agagaacctc ttttaaacgg caccttggtg gggcctgggg gaaaggttat ccctaatttg |
| 5161 | atgggactct tttatttatt gcgcttcttg gttgaaccac catggagtca gtggtggagc |
| 5221 | ccaggtgtat ctgggaaatg ttagaatcag gtgwgttgtt aaacctgtca gtggggtggg |
| 5281 | gttaaaagtc acgacctgtc aaggtttgtg ttaccctgct gtaaatactg tacataatgt |
| 5341 | attttgttgg taattatttt ggtacttcta agatgtatat ttattaaatg gatttttaca |
| 5401 | aacagaattc tgatcactgt cttcttcggg cagctgtggg actcctacac tgagagtcat |
| 5461 | tcgaacccca agtggaggtg gaggtggaga attgtgtggg agcatttacc acagccaacc |
| 5521 | acggaactct ttcagagaac agcttctcac accgtctaca ccagcctccc ggccaggctt |
| 5581 | tgcaggcagc cccaggccca gtgcgtggga ggggaggctg ttgcaaggtg ataggaaaca |
| 5641 | ccagtttcag gcttggggtg gcagcaagtt ggttggccta cagctggaag gctcttcatt |
| 5701 | gtcgcttgct ttcatcttcc tggtttaaat tcagccagga ccttacttct gctttaggaa |
| 5761 | gctt |

Two SNPs:

| Position | 4476 | 4679 | Estimated frequency |
|---|---|---|---|
| | C | A | 48% |
| | C | T | 26% |
| | T | A | 26% |

Three SNPs:

| Position | 4476 | 4679 | 4909 | Estimated frequency |
|---|---|---|---|---|
| | C | A | T | 10% |
| | C | A | G | 38% |
| | C | T | G | 26% |
| | T | A | G | 26% |

PBE64

| | |
|---|---|
| 1 | tcaggctgtc accttttatg aaaattttat aaagttttga aaaaagaaga aagaaatcta |
| 61 | tcatgggttg ttgaaagttt tatattcaga attaattgta taatgtaaat ccaaRataca |
| 121 | taacatttaa aatctaccca tatatagagg gatataagtg gaagtaccat agctgtaaac |
| 181 | acttgagtat agataattat tttaacttaa tttctcccat wctttttaaa gacatgacag |
| 241 | caagtacrar aaacaaacaa acaaacaaaa ycagagtatt gtgcaggtat atcaatagcc |
| 301 | ctcaaggaaa gaaacgattc cagcattact acaggatgaa gtctttgcaa caataaacac |
| 361 | aaaaaattga ctgaatgaca aaacagaatt ggattttctg tgtctgacac agaatttcSa |
| 421 | tcttcaaata gatgcctctg ggtatatttt tccaaatgtt gcccaacaat tttattcata |
| 481 | aatatcacac tttgaaaatt cacctgctgt acctYaaaat gataatctaa taaaggaagg |
| 541 | acagaaaaaa tactgcagga tgctcagata gacctcctag gacttaacta aatacatcta |
| 601 | acaaattgaa tcagaattat cattacttga cagctttgta tttgattaca aataattacc |
| 661 | aaaacaccca gtaagatctt gcttttcaaa ttatgtaaca ttccrttaca cactaa |

Single SNP:

| Position | 515 | |
|---|---|---|
| | C | 39% |
| | T | 61% |

Two SNPs:

| Position | 419 | 515 | Estimated frequency |
|---|---|---|---|
| | G | C | 39% |
| | C | T | 27% |
| | G | T | 34% |

Three SNPs:

| Position | 115 | 419 | 515 | Estimated frequency |
|---|---|---|---|---|
| | G | G | C | 39% |
| | G | C | T | 27% |
| | A | G | T | 6% |
| | G | G | T | 27% |

SCAMP

| | |
|---|---|
| 1 | cattgagatg aactgaggag ctgttgataa tgaatgtata gatgaccact taccttctcc |
| 61 | cacttttttg tgcctgtagg tccatggact gtatcgcaca acaggtgcta gttttgagaa |
| 121 | ggcccagcaa gagtttgcaa caggcgtgat gtccaacaaa actgtccaga cggcagctgc |
| 181 | aaaYgcagct tcaactgcag caactagtgc ggctcagaat gctttcaagg gtaaccagat |
| 241 | ttagagagtc ttcaaataat acactgttac cttttgactg tacttttttc tccagttact |
| 301 | gtattctata aatatttttt tgttcaaaac acacagtaca cacagcacgt atatttccta |
| 361 | atcacttgtg catgggctaa aaccagaaRa acttcgttgt cttattattt acctgacagt |
| 421 | ttcttaatct ttcagtgccc cttgcaggaa aaaaaaatta catgctaaat aaatattctc |
| 481 | catatttttt gggggatgaa tgttcagcaa attttYtcgg tggtgacaca ctgaaatcga |
| 541 | catggcattt aggattaaaa atgcacttag tacttgctgc aRtcattctt tcaagagtct |
| 601 | tagacataag gattacacac tggagcagta aagcaatgct tcattccttt tctttatttg |
| 661 | tattgaaaga aataggacat cagaaactta gggactttta aattggcttg ctttttagca |
| 721 | gtttcagtca ccagtgaaga gcctatgtgc atttcatagt agataatgta aattttatct |
| 781 | ttttattttc tttttctaga gtaattgata ttttgatatc aatctctgat cttgcatggg |
| 841 | caccatgttt cctaaaaaaa ytagtatttt gggttatgca ctgcttctgg ttgtaggatt |
| 901 | ggggagtttg tagaatcata aaaatgattt tctgtaatWg tttcttttaa ataaaaattt |
| 961 | attggagtgc aatatgagga tataatatac agtgcattat ccaaaagaaa aagtagataa |
| 1021 | ttgatg |

Two SNPs to score:

| Position | 389 | 582 | Expected frequency |
|---|---|---|---|
| | G | G | 15% |
| | A | G | 50% |
| | A | A | 35% |

Alternative two SNPs:

| 582 | 939 | Expected frequency |
|---|---|---|
| A | T | 33% |
| G | A | 26% |
| G | T | 41% |

Three SNPs to score:

| Position: | 389 | 582 | 939 | Expected frequency |
|---|---|---|---|---|
| | A | A | T | 33% |
| | A | G | A | 26% |
| | A | G | T | 26% |
| | G | G | T | 15% |

Alternative three SNPs:

| Position: | 516 | 582 | 939 | Expected frequency |
|---|---|---|---|---|
| | T | G | A | 26% |
| | T | G | T | 22% |
| | T | A | T | 33% |
| | C | G | T | 19% |

SNP at position 184 (c/t)can be included.

LEPR (Leptin Receptor STS7)

121 ttacctggaa atttcttcag cttgcctcac tactaaatat ttatttcctg taactgtctt
181 ttattgcata tgatttgttt tattggcttc aaagcatatc ctcctctatt ctgtcgctct
241 tcctgttaaa tagattgaty taattctaac ccctttaagg aatgaaattt cctaaaattt
301 atcatttccc aaagtgtgtt ttatagaaca ttgatttcat aaaattgttc ttaaaaaaga
361 ttacatgggt aaataaagtt taggaaaccc tacatcactg tatgtccaca gtgtagaatc
421 atcttVtata ctaaaggttt ggagaagccc tgaattaaag aaacatttgt gactttgttt
481 catcctatgt tcctcaaact tattttacca aagaaccttt tctcctctaa ccatattctt
541 tagggcgtat gtgttccttt gcatacattt tggaagaagc tgcttttatc aatcagaatc
601 atacctatag ttgcaagcat atgtatgatg acttgctgtg tcatttttct gatggcagtc
661 tgcaaagact tacaaatagc agaaactctt aattatgtca ttagatcata atgacttcag
721 ctgaaatgaa tgtgacagtt tacttgctta tagaggagac tatcgagaga ttctctacag
781 caggccctgt ctaaccacag gttaaaattS ttaaaagtct ttgtggatag aggattagtg
841 gacarggatt agcaatgggg ttaagagaaa tgattgggaa gtgacacatt gcagtgagcc
901 agtccaaatc ttgtcatgaa atggaaataa caagatgact aaatggggga aaaatgtaat
961 tgtaatgtat acatgtaagg ataacctgac

Two SNPs:

| Position | 426 |
|---|---|
| | A |
| | G |
| | C |

Three SNPS:

| Position | 426 | 810 |
|---|---|---|
| | A | G |
| | A | C |
| | C | C |
| | G | C |

COX2

| | |
|---|---|
| 1 | aatctggctg cgggaacata atagagtgtg cgatgtgctt aaacaggagc acccggaatg |
| 61 | ggacgatgaa cggctgttcc agacgagcag gctgatactg ataggtgcgc aagaacaact |
| 121 | cttctcaata acgctcttct ccagggaaaa cgaaactgtt tctttgcagt ttccagaaat |
| 181 | rctgggggta tgtggtgcat gtaaaatcac atgcttcata gtaattcaac ccytgggctt |
| 241 | gattaggaat atcaccgacc ttttgtttyg atggtaaaaa aggaagacac agaaatcaat |
| 301 | agaatatggc aaattaacaa aattgcattt gggttgcttg aaagtttgtg agtagaaaga |
| 361 | atttgtgYtc taaatytgtt aatgttgtgc ccataggaga aacrattaag attgtgatcg |
| 421 | aagactaygt acaacacctg agtggctacc acttcaaact gaagtttgac ccagagctgc |
| 481 | ttttcaacca gcaattccaa taccaaaacc gtattgctgc tgagtttaac acRctctacc |
| 541 | actggcatcc ccttctgcct gacgccttcc agattgatgg ccacgagtac aactatcaac |
| 601 | agtttctcta caataactct atcttactgg aacatggcat cacccaattt gttgaatcat |
| 661 | ttagcaggca aattgctggc agggtaagca ttattattat aaaacgaaac aaagggctta |
| 721 | gtcagtaact ggaatttctg ctgtagaaat gatttttcgt aaacgtatta aaacagtaat |
| 781 | tatttgctag tagaattctt cccttaaaat gagaagtcta atatataatt tcggttatag |
| 841 | taaatgttat cactataatc tagatgacag aaatattctt gaacagttta ggtctcagct |
| 901 | gggagctgag tcttaccttc tttgtaccca agggatgcYt ttaaaataga aatcttaaat |
| 961 | atacctaaaa ctcatgttct acaatttcat ttcatttcca caggttgctg gtggtaggaa |
| 1021 | tcttccagct gcagtacaaa aagtatcaaa ggcctcaatc gaccagagca gagagatgag |
| 1081 | ataccagtct tttaaa |

Two SNPs:

| Position | 368 | 533 | Estimated frequency |
|---|---|---|---|
| | T | A | 54% |
| | T | G | 17% |
| | C | A | 29% |

Three SNPs:

| Position | 368 | 533 | 939 | Estimated frequency |
|---|---|---|---|---|
| | T | A | T | 54% |
| | T | G | T | 17% |
| | C | A | T | 12% |
| | C | A | C | 17% |

P450-STS18

| | |
|---|---|
| 1 | caagagcgtg tcgctgctgg gaaggaaccc tgctctccac cgccaccctc tctctcagga |
| 61 | ccctgtgggc YRgggctcca cctcctcacc ctgagaaagg gaaccatgtc caaaatttgg |
| 121 | atggaccagt gctcccaRgt tttcatcagg tcctggacac agtcgtgaag ggcatgcact |
| 181 | aaggtgtcct cctgccggaa atggaggaaa tcctttcaga tcaggacctg gagaaggtca |
| 241 | ggcagcggct gaggggtggg tccaggcaca tgtgaaggca agagccttga ccttgtctcc |
| 301 | aaaggtgagg caacagatga tgctgcaggt gaggacagag aattccttct ggatggtcac |
| 361 | Rggggtcccc gcctgggctc tcatgcgctg tggggaggag catgaactca gtagggggcc |
| 421 | tgccaggagg gggaagctgg tgcaggatgg ctgagggggt ccagcctcac ctcacagaac |
| 481 | tcctgggtca gctgctccac ccggggctcc atggagctgc ggacgcccag cagcagggct |
| 541 | gagcgggtga gtttcttgtg agcyttccag aacagrgagt artcccctag cgagatgtcg |
| 601 | gggcagtgct gagacgccag cttgtctgtg agtaagggtt gggggcgggg gttcgaactg |
| 661 | accaaaggaa gtcacggacc tgaccttccc cgcctcctgc agcccctgcc ccttccttca |
| 721 | gaaagagccc caccccttac aggatggtat ctggggtcct gccgg |

Three SNPS:

| Position | 71 | 72 | 361 | Estimated frequency |
|---|---|---|---|---|
| | T | G | G | 21% |
| | C | A | G | 23% |
| | C | G | A | 16% |
| | C | G | G | 40% |

AMG

| | |
|---|---|
| 1 | tgtaagggaa ggttctgcca cagttctctt cttgctggta tttcctgctg gtgtgaggga |
| 61 | aacatattgc tcttcccgac atggagtgag tttcatcaag aaataaaagg aaacaaaaaa |
| 121 | aatagaagaa caaagaaaag gagttctctg tggcgcggca ggttaaggat ctggtgccgt |
| 181 | cactgcagcg gctatggcct ctgctgtggt gcaggtttga tccctggccc gggaacgtcc |
| 241 | acagactctg ggcacagctg aaagacagac agacagacag aaggaaaatg atgggtgggc |
| 301 | tagagtagga ttaactgagc acaggggtgg gaggggtgtc tgaggatgac cggaggataa |
| 361 | ctgcatgctg gtttctgctt ccgctgtaag gttacaacct ctgggaaaac catttcgttg |
| 421 | ctctggccct ctttaagata agagggctcc tctcctaccc agcatacatg ttccaactaa |
| 481 | aagtagacct tcaagatatt ctgcactata tagattttgt aaaagtagct tcggtctctc |
| 541 | ttaatgtgaa aattgcatat tgacttaatc tcttcccctc tctctctccc cctcccccct |
| 601 | tccctcttcc cttgcacccc ctcactcttc ttcttctcct ttcccccttc ctataaaagc |
| 661 | taccacctca tcctgggcac cctggttata tcaacttcag ctatgaggta atttttctct |
| 721 | ttactaattt tgaccattgt ttgacttaac aatgccctgg gctctgtaaa gaatagtggg |
| 781 | tggattcttc attcaggatg tttgtcagtc ccattttttc agttctcact gccagcttcc |
| 841 | tagtttaagc cctgatgggt cacctcaagc ctgcattgcc ccagaaccct cctacctgcc |
| 901 | ccccca(A)cccaacccccgactcagtStctcctccgtatacg gctgtaaaat gaacaccccc |
| 961 | tggagggggg acgRcatggt agggcagaaa ctgaactctg gctgaMcaga gttctatccc |
| 1021 | ggcctggaaa atatggggac tcaggtaaga tgttatcWac ctaaggtcct tKccagccag |
| 1081 | accactcctg gttctaagac gtgcacactc tacgtgtctc cctYgctggt ctttcggaaa |
| 1141 | gatgagcgac caagggggct gtgtgacatt ctgccgagca agggaaagta tgagatggct |
| 1201 | ggaaatcagg tttgaggcgc ttctcatgcc cacacgaacc atgggacctt gggcaaatca |
| 1261 | ttgtctctct ctggaacttt ggtttcttca tctggaaaag ggaaatgatt ataataccca |
| 1321 | acaatttaaa atattgattg gggagcgaaa gagttaagca acataaaagg tgctttgttc |
| 1381 | agtttgcctt gagcaaggtc gtaattacgg tattgctatc aaatgcttat tactgtctga |
| 1441 | aggagtccct ggacctgagg ttactcGagt ctatacggtt aaggaaggaa ggaagtgctg |
| 1501 | acttctttcc tcggttcaga tgacaaccca tgggtatgtt gactcctaca agctgaggac |
| 1521 | aagggttaac aaaaatccga ggaaagattt tctgttaaat ctgaaaaggt tgacatatgt |
| 1661 | aaccggcaaa cgcgtttcta ggatgagaaa ctggtttggc ctccttaata tttttgtgac |
| 1681 | atcagatcaa aagaggttac aattcctgtg aggtcacatt aattctctgt tttgtttttc |
| 1741 | tcttgcaaag aagagcgggc gctggggagc gagacttact gcttttgtaa gctccgtcca |

Single SNP:

| | | |
|---|---|---|
| Position 906 Indel | A | 37.5% |
| | C | 62.5% |
| Position 1467 | G | 50% |
| | A | 50% |

Two SNP assay:

| Position 906 | 1467 | Estimated frequency |
|---|---|---|
| C | A | 20% |
| C | G | 37% |
| A | A | 42% |
| A | G | 1% |

Single SNP at position 975 could be substituted for the indel at 906

| | |
|---|---|
| 975 | |
| A | 56% |
| G | 44% |

Single SNP:

| | | |
|---|---|---|
| Position 926 | C | 73% |
| | G | 27% |
| Position 1006 | A | 36% |
| | C | 64% |
| Position 1058 | A | 63% |
| | T | 37% |
| Position 1072 and (1124) co-segregate | G(C) | 59% |
| | T(T) | 41% |

CTSL

| | |
|---|---|
| 1 | ttgatgaagc tttcttcatt cacgagagaa agtcacaatt tgataacctc cagaaaccac |
| 61 | aggagcccat cagaagacta cccaaagtca gatgatctct agattgaagg aaagcaggcc |
| 121 | tgatccttac cagcaaccct gacccttgaa ccttgatggt aagaatcctc agaaatctcc |
| 181 | aggttatgtt tgtttgtttg ttttggccat gcccacagct tgtgaaaatt cctgggccag |
| 241 | ggtttgaacc cRgcatcaac agtgacaatg cRggatcctt aacctgttrc accacaaggg |
| 301 | aactctaagg acacagggtt ttgagggcat tcgtccactg tgtccctctt tgcctggcaa |
| 361 | agcaataaag ctcttctttt ctaccccact caaaactctg tcctcccaga ttcaattcgg |
| 421 | crtccatgca cagaggccga gttwccccat cagtattgga gggaattgtt aagcggcttc |
| 481 | agggkctttt tttttttttt tttttttt |

Single SNP:

| | | |
|---|---|---|
| Position 252 | A | 37% |
| | G | 63% |

Single SNP:

| | | |
|---|---|---|
| Position 272 | A | 49% |
| | G | 51% |

Two SNPs:

| Position | 252 | 272 | Estimated frequency |
|---|---|---|---|
| | A | G | 31% |
| | G | G | 20% |
| | G | A | 50% |
| | A | A | 6% |

LCN

| | |
|---|---|
| 1 | cccccacggg gtggggcaga gtctggggct gcagagtcgg ggtaggggat cagccggagc |
| 61 | ctgatgggag ggcctttctc cagctgYtgg ggagatggta tctgaaggcc atgacctcgg |
| 121 | acccggagat tcccgggaag aagcccgagt cggtgacccc cctgattctc aaggccctgg |
| 181 | aggggggcga cctggaagcc cagataacct ttctgtgagt gtcgcctccc gccttcccct |
| 241 | ccccgcacca ggagggcggg ggtctctggg gtgtccttct cagccccttg tgtgacactt |
| 301 | agccctggac agctctgggg ggaaccgtcc tagaggggac agaccccgga tgagaccctg |
| 361 | tgggtgggag ggScagtgct gggagaccca ggcaactgcc aYRtgccagc tgatgcctgg |
| 421 | cctggaggtg gctgacacgc catcgtccct cccccctccc ccccgggcta ccacggaccc |
| 481 | caggctgcct gtggctgctg ggccaggggg accggagccg gggctgggcc gggtctccaa |
| 541 | ggtgggtgac ccccaggcag catcacacgt ggcttctgtg ttccaggatt gacggtcagt |
| 601 | gccaggacgt gacactggtc ctaaagaaaa ccaaccagcc cttcacgttc acggcctgtg |
| 661 | agtctcgggg ccctggccgg gggcaggggt gggggcggcc agcgagtttc tgggacggtc |
| 721 | ttgcagcctg aggagcccta ctgctttctg accctattaa atgccaccct ctcctcccac |
| 781 | tggtccattt gccttRatga tatgaacccc Ygacgccagg cgtgacggat ttgccctcgg |
| 841 | ggggactggt ttgtcccgag ctccagctgg gggtcatagc tgtgccaggt caggcccagg |
| 901 | agcagagcct ctttgagccc ccctcccccg ctgtcaccgc cggttggggt gtgaccacct |
| 961 | ctccagtgtg ggctctcccg ggacgtgggg gccccacagc ctggggggtc ggctggggtg |
| 1021 | gaagcccggg gcasgtgccc cgggagggtc ctgggcttac cgggaccggg cccgtctccg |
| 1081 | ca |

Position of SNP based on this sequence:

| Position | 87 | estimated frequency |
|---|---|---|
| | C | 54% |
| | T | 46% |
| Position | 373 | estimated frequency |
| | G | 66% |
| | C | 34% |
| Position | 402 | estimated frequency |
| | C | 77% |
| | T | 23% |

Observed three SNP combinations

| | | |
|---|---|---|
| C | G | C |
| T | C | T |
| C | S | Y |
| T | C | Y |
| T | S | C |
| T | S | Y |
| Y | G | C |
| Y | S | C |
| Y | S | Y |

Estimated frequency of all possible combinations

| | |
|---|---|
| TGT | 6% |
| TGC | 22% |
| TCT | 13% |
| TCC | 5% |
| CGT | 3% |
| CGC | 43% |
| CCT | 2% |
| CCC | 6% |

MYF5

| | |
|---|---|
| 1 | cgccttccaa aacggggttt cttagaacac acagcttaga ggtgcagtca gtggggctcg |
| 61 | tccgaccgat ctggggccat ccaaggcctg gcgcacgtgc agagcgggaa cgcgaggggc |
| 121 | cggcgtgttc gcggtcgccc ggcccgcctg tgcccaggcc cctcggagga gcagagggag |
| 181 | aagtcggagc ttcggagcga gcccggcctt gggggaaaga gcctctccgc tccccccaat |
| 241 | aacacagagc ctacaaaacg gcaggctgat gagaacagaa gagactttaa aaaaaaaaaa |
| 301 | aaatctaaat ccactccatt actgaaagtg ccatttcaca attttagtgg gccgttatta |
| 361 | ggcccactgg gtgaaaaaac aaattcttcc cacaactgaa gtgcatgaaa aagataaaca |
| 421 | tctaaaagta aagccatctc tcctgtcccg gattgagaag gcaggtcagc ttgtgtcagc |
| 481 | tgaaagtagg gagttctatt tactactttt tttttttttt ttttaatgtg ggagcaatgg |
| 541 | caaaacgttg gatttccttc gttcctttgg cctgtgacac cctttaagcg tcccttgatt |
| 601 | tgctctaaac agcgcaagta agcggggtgg gggagccgtc accccgcccc agcagaaagg |
| 661 | cagtaaaacc attagcgtcg aagggccggt aaacagccca ctgtctctaa aggaaaggcg |
| 721 | gaggtttgcc caaacagccc cggcgggggt tgcggtggga tatgctaata gtgccgggcc |
| 781 | actggggccg gcctccctcc cccaagaaca tataaagggc cccaacccca gcgcggccga |
| 841 | cccaggccgc caggcgtctg cccctgttaa ttagcagagc aaccgagcag ggagttccgc |
| 901 | ccgcgacgtg cccgcccgcg gaggcgccag gccccgggct tctccccgat ctgatctatc |
| 961 | tcgcagctgc ccaggtgcac cgcccgcctg tccgcagaag atggacctga tggacggctg |
| 1021 | ccagttctcg ccttctgagt acttctacga tggctcctgc atcccatcccccgagggcga |
| 1081 | gttcggggac gagtttgagc cacgagtggc tgctttcggg gcgcacaaag cagacctgcc |
| 1141 | cggctcagac gaggaagagc acgtgcgagc acctacgggc caccaccagg ccggccactg |
| 1201 | cctcatgtgg gcctgcaaag cgtgcaagag gaaatccacc accatggatc ggcggaaggc |
| 1261 | ggccaccatg cgcgagcgga gacgcctgaa gaaggtcaac caggcgtttg agacgctcaa |
| 1321 | gaggtgcacc acgactaacc ccaaccagag gctgcccaag gtggagatcc tcaggaatgc |
| 1381 | catccgctac attgagagcc tgcaggagct gctgagggaa caggtggaaa actaatacag |
| 1441 | cctgcccagg cagagctgct ctgagcccac cagccccacc tccagctgct ccgacggcat |
| 1501 | ggtaagagaa agctcgggac ctcctaggcc cttctaatct tttccaaaaa actttacctc |
| 1561 | tcgtttaagc caggtgtagc aaccgaatat tctgatagtt ggctgtgggg gtgaggaggc |
| 1621 | agttgcccta agagagatgc cccatttaga cagacgccag gaaaccgctg ctgaagagca |
| 1681 | taatactttg cctcccaagt tctaggtgaa cgttgccggg ggaggttctc atgtgagacg |
| 1741 | ggtggctgtg aatgatcaga ggttttctcc attactcact ttacttccga tatatacccc |
| 1801 | ccgtggaccc caccgtacac taacgtttaa agRcaactga cggaggctcc ccatagcgca |
| 1861 | tggtttctaa ctctaggcag aaatgggatg aaacacccgc atggccccgg ttgctgctct |
| 1921 | gctgaggcct ggctggaaga tgttgatgca ttcttttcag agggtgtctg ctctaacgct |
| 1981 | gccaggtttt aatgtgtttt tgccctggga aagtgttctc tctccgaatt agtgtggctt |
| 2041 | ccttccaccc caatccattt tgcatggtta acccagtgca cgttgctgcc gaattccacc |
| 2101 | ccgcccctct ccttttctcc cagtacagtg actgacctca gcgcccttgc catttgggga |
| 2161 | ggcgagccct tcctaaatca aggcagtgaa ggtgactgag agtSgtcaac tttcgaagct |
| 2221 | ggagggcaag cactgcctca ccctccatca gagcaccttt cgccaagacc tgaaaacaaa |
| 2281 | tgccttcttt tgtgtctttt attatagcct gaatgcaaca gccctgtctg gtccMgaaag |
| 2341 | aacagcagtt ttgacagtat ctactgtccg gatgtaccaa atggtaagaa cgaacgcctt |
| 2401 | tagaggaggt taaagaccag ttcaacttca cagttcagcc catcaaatca gtctgtcctt |
| 2461 | ccaggcagtt atctggagga aaagagaatg gtttttacag ggactttttg gcggagcaaa |
| 2521 | ataaacatct gctcaaaatt cccctcagag atactcccat gcacacacac aggcacatga |
| 2581 | gcttttgctt aaaacattac cgagggtgct tttccactcc ccacctgcac ccccatgaat |
| 2641 | tgctagatat atatgttgca aatttctaca ctggggtctc tgtgaccacc tgacctctgg |
| 2701 | gtttcaaagg agctgacctg cagttcaaag ggcaacgtaa gcaagtctac ctattgggtt |
| 2761 | tttttttttt taacgttttt ttttcccctt gtatctttag tatatgccac ggataaaagc |
| 2821 | tccttatcca gcctggattg cttatccagc atagtggatc ggatcagcaa ctccgagcaa |
| 2881 | cctggactgc ctctccagga cccagcctct ctctctccag ttgccagcac cgattctcag |
| 2941 | cctgcaactc caggggcctc tagttccaga cttatctatc acgtgctatg aactaaaaat |
| 3001 | ctagtctaga ccatttctgc caggagtgcc tattacacag gaggaaggag gcccaaaagg |
| 3061 | cccaaaagca agacaacctg tatataaaca ttttttttca gttgtaaatt tgtaaatact |
| 3121 | atcttgccac tttataagaa agtgtattta actaaaaagt cactattgca attaattctt |
| 3181 | tatttcttct tcttttcctt tgtcttggca ttaaatatat agttccaatg atattatttc |
| 3241 | ttataggggc aattcatcca agggtagctc gttgcaatgc ttaacttata ctttttataa |
| 3301 | tattgcttat caaaatatta cctctgttta gagctttatt tttttcccct ttaaaaatat |
| 3361 | tagaacaaat actagaactg gaaatcaagt tatagggagt tttaaatata tttaactttt |
| 3421 | ttgcttctct ttaatccttt ggttatattg tgttaagtaa aaatataaca tactgcctaa |
| 3481 | tggtatatat tttgatctta taagaaatgc atctttttaa tgtaagcaca aaatagtact |
| 3541 | ttgtggatga tttcaagatg taagagattt tggaaattcc accataaata aaattgttta |
| 3601 | aatgaagaat catttgattt atgattttgt taaaagaacc tctaatagca ttggcagtga |
| 3661 | ttgatacgta tctttgagct |

One SNP:

| Position | 2204 | |
|---|---|---|
| | C | 48% |
| | G | 52% |

Two SNP

| Position | 1833 | 2204 | |
|---|---|---|---|
| | G | G | 42% |
| | G | C | 53% |
| | C | G | 5% |

All single SNPs added to 2204 to make a 2 SNP combination result in roughly equal results.

Additional SNPs:

| | | |
|---|---|---|
| Position 2425 | A | 90% |
| | G | 10% |
| Position 2453 | C | 98% |
| | G | 2% |
| Position 2513 | A | 13% |
| | G | 87% |
| Position 2568 | C | 86% |
| | T | 14% |
| Position 2640 | C | 12% |
| | T | 88% |
| Position 2678 | C | 90% |
| | T | 10% |

PBE42

| | |
|---|---|
| 1 | atgtctccat ttcttttgaa gacaKatgag ayctgagaaa aggtgatgct ccctgatgta |
| 61 | gtgggctcag cagtgtctga gcagaactct caggacaacc tgtctRagac rtccaacKtg |
| 121 | atagcaaagc tgagacaaaa tgccacctgc tgactcaccg caggagacag caagctggta |
| 181 | Ygatatcatt agagaaataa caacacacaa caatagcggg cacttgttgg gcacttcctc |
| 241 | tgaaggcagt rtgccaggag cgatcttcac atcttcattc aatccccagg acaacctgtt |
| 301 | atcagaggct tcacgatatc cgccttcatc cataaaataa agtatacaac atgctcagca |
| 361 | ctgatgggac agagagaagg tagggtccca catgtctagt ctttaattcc acatcatgtg |
| 421 | acaccaggct acaagaaaaa tctgaaggca gaaggcagat ctggagatct agctggccag |
| 481 | gtttcctgga a |

There is a complex In/Del/SNP combination at position 7. This region should be avoided when designing primers.

| Position | 111 | 118 | 181 | Estimated freq |
|---|---|---|---|---|
| | A | G | C | 48% |
| | A | T | C | 8% |
| | A | G | T | 27% |
| | G | G | T | 17% |

Additional combination 2 SNPs

| Position | 118 | 181 | Estimated freq |
|---|---|---|---|
| | G | C | 48% |
| | T | C | 8% |
| | G | T | 44% |

Additional combination 3 SNPs

| Position | 25 | 118 | 181 | Estimated freq |
|---|---|---|---|---|
| | G | G | C | 48% |
| | G | T | C | 8% |
| | T | G | T | 13% |
| | G | G | T | 31% |

PBE57

| | |
|---|---|
| 1 | cacactcacg cctcagaccc catcgctgtg cagtgacatc ggcattgatg rtggccggtc |
| 61 | acycctgcac cagcYggccc ctcccatttc tccagggaca agawgtagRt ttgaggttcc |
| 121 | tcgtctgact tcagcctcccc(c)trtttcctcactcrctgt cccgtccctt ctccgtccct |
| 181 | gtttctggaa gycgccYtcc atgaccagga cactcagatt cttacttctg aggattatac |
| 241 | tgaaaccctt gccactccct tcctgtgkcc tgttcatcac tgcgtcacct gaccacccca |
| 301 | gcaccctctc cctrcgcygtgc(agacacc)ctgttttgcrc tgtccccagc agctggcagg |
| 361 | aggaggtctc atcacagagc agccctggcc caccgagtcc ccacctrccc aagaacacgg |
| 421 | ccctcactct gcaactggcc acgccgctgc tcaccaccct tgcctygtgc caggacccga |
| 481 | gagmgcactg gtccttgtga gtggygtcct ctgccttcaa gcatcagttc atccatctcc |
| 541 | ccacacacac gag |

The sequences in ( ) are In/Dels.

Two SNPs:

| Position | 75 | 197 | Estimated frequency |
|---|---|---|---|
| | C | T | 50% |
| | C | C | 28% |
| | T | C | 22% |

Three SNPS:

| Position | 75 | 109 | 197 | Estimated frequency |
|---|---|---|---|---|
| | C | A | T | 50% |
| | C | A | C | 12.5% |
| | C | G | C | 15.5% |
| | T | A | C | 22% |

Additional SNP at position 268

| | |
|---|---|
| G | 80% |
| T | 20% |

PBE73

| | |
|---|---|
| 1 | tttctactac yngscatcat ttcccttccg actgtaggat ttaactttaa catttccttk |
| 61 | gtacaggtct gctggtgatg aacgctttct gcMtatttat gcctgaggaa tctttRtttt |
| 121 | gtcttcatta ttgaaagata ttcctgctct gtatagaatt ctacactaat agacttYgct |
| 181 | ttcagtaggt acttcggaag atgctgctcc actgtcttct ggcttctttg tttcctatga |
| 241 | gaattctgcc atctttatct ttgcttctct gkatggaatg tgtcattttt ttcctcagac |
| 301 | tgctaataat atattctctt tatcactggg ttccagaaat ttcattctga tgtgtctkgg |
| 361 | tgtaattttc tttacatatt gtgtgcttgg ggatganctg tgattcttag atctatttat |
| 421 | agtttttata aaactttgga aaattcagca aattcagcaa ttaatcttct ttttttYtcc |
| 481 | tgttgttcct tctccccatt ctccttcaga accgcaatta tatgtacatt taggsccacs |
| 541 | ctgaagstat ccta |

Positions of possible SNPs based on this consensus:

| position 93, | frequency |
|---|---|
| A | 80% |
| C | 20% |
| position 116 | |
| A | 89% |
| G | 11% |
| position 177 | |
| C | 87% |
| T | 13% |

Three SNP estimated frequencies

| | 93 | 116 | 177 |
|---|---|---|---|
| A | A | C | 61% |
| A | A | T | 5% |
| A | G | C | 10% |
| A | G | T | 1% |
| C | A | C | 22% |

Other potential SNP positions:

| Position | 375 | Indel |
|---|---|---|
| | − | 86% |
| | + | 14% |

| position | 477 |
|---|---|
| T | 96% |
| C | 4% |

PBE84

| | |
|---|---|
| 1 | caaaacttct aacRtgcaga aaaagcggKa gartttacag tgataaacac ttgcattaaa |
| 61 | gaaaactaaa gatctgaaat aaacaaccta actttaYacc tcaaataact agaaaaagag |
| 121 | gaataaacta agcccaacat tactggaagg aaggaaataa taaagattat ttaagagagM |
| 181 | agaaataaat gaaattgaga ttagaaaaca atagaaaata tcagcaaaat taagagctag |
| 241 | tttttcaaaa gattaaaaaa aaatgaatct ttagagtaag aaaaagagaa aagatgagta |
| 301 | aattaaacta taactgaaag aggagatgtt accaactgac actgcagaaa tacaaaggat |
| 361 | cccaagagac tactatgaac aattatgggc caaaaaaatt gggtagcttg gaagaaatgg |
| 421 | ataaattYct agaaacatag atcccaccaa gaggcaaata tgacaaaara gaaaatctga |
| 481 | acagaccaat aatgagtaag gagattcaaY caattaaaaa aaaaaactcg ctaatgtaaa |
| 541 | gcccaaaact aaatggtttc actgctgaat tctaccaaat Wtttaaagag ttttaa |

Two SNPs:

| Position | 29 | 97 | Estimated frequency |
|---|---|---|---|
| | G | T | 50% |
| | G | C | 28% |
| | T | C | 22% |

Three SNPs:

| Position | 29 | 97 | 428 | Estimated frequency |
|---|---|---|---|---|
| | G | T | T | 10% |
| | G | T | C | 40% |
| | G | C | C | 28% |
| | T | C | C | 22% |

Alternateive SNP:

| Position | 14 |
|---|---|
| A | 48% |
| G | 52% |

PBE 112

| | |
|---|---|
| 1 | ctgctccctc cacaccgtct ctttctttgc gggtcggtgg gttcctctta gggacccaga |
| 61 | Yaacttgctc ctccctacct ctcatcYctg aaacccgaat ttctgtccct ggcctggcag |
| 121 | ccgtccaccc ccagccctcc Yagctaaggc gtttaacatg cctgcccgca gtatggtggt |
| 181 | ctagaaactg cccaggagcc agggggttcc ctgcaaattc tgtcccaggc tgtggcccca |
| 241 | gctgggtggg ggaaagggac ctgtaagctg caagataagg ggacctcagg tgtggggagt |
| 301 | tgttgatgtt ttcacctaca agtgtacctg tttgctggct tgtgtgtatt cccacctgtc |
| 361 | tgcctgggac tctgaaatag tctggcctgg ggtttctaac cctacccccc aaggtgaggc |
| 421 | ctccaggttg gggtgggaag tctaatcagg tttccccatt cctgaaaatg gggctgtagt |
| 481 | gagttgcagc catttgctca aggccttctc aggagtggga ggcagcggga atcagaggaa |
| 541 | acagtgaatc aaagagactc caaagagggc ccatctacac tgacagaaga actagcagcc |
| 601 | cgagagtaaa tccaacccct gttaatcctg tttgatgtct cttggaRtga tgcaagtcat |
| 661 | tgagacactc act |

Position of SNP based on this sequence:

| Position 61 | Estimated frequency |
|---|---|
| C | 34% |
| T | 66% |

Alternate SNPs

| | | |
|---|---|---|
| Position 87 | C | 94% |
| | T | 6% |
| Position 141 | C | 90% |
| | T | 10% |
| Position 647 | A | 94% |
| | G | 6% |

87 and 141 cosegregate, either could be used.

Two SNP assay

| 61 | 87 (or 141) | |
|---|---|---|
| C | C | 34% |
| T | C | 60% |
| T | T | 6% |

PBE132

| | |
|---|---|
| 1 | gcggtcgggg ttgggtagca catcctggcc tcttcctgtt ggccccacag gctgagggag |
| 61 | atgtcacttc ctgctgttgg gaggtgaccg gcaggatgga Sgggagcagg aaggggtggc |
| 121 | cgtgaRccag cactaaccag cctgggccat ttcctcaccc cggaaagagg gaaggagaga |
| 181 | gagagggaag acYggtgttt gggagtctcc tgtggccctc acaccoagag rcagtgggtg |
| 241 | ctcagacccc tgctcccacg ggcactgatc tggcacaggc aggcgttcag aaaaaaaaaa |
| 301 | ctttcaaaac aatcctatgc actggcccct gcacgtcaca gtttccaatg gccttctcan |
| 361 | ccccggcctR agggctttcc tgccatccct ctgactgtag gacagagttg ggggattatt |
| 421 | gctgccaccg gaaaatgagg aanctgaggc ccagtgaggt tgcaatganc agtctgccct |
| 481 | ggatygtaca gcaaattant accaggactt g |

Single SNP:

| | | |
|---|---|---|
| Position 102 | C | 53% |
| | G | 47% |

Alternate SNPs:

| | | |
|---|---|---|
| Position 127 | C | 53% |
| | A | 63% |
| | G | 37% |
| Position 194 | T | 63% |
| | A | 37% |
| Position 371 | A | 58% |
| | G | 42% |

4 SNP observed combinations

| | |
|---|---|
| CATA | 30 |
| GGCG | 18 |
| GRYG | 4 |
| GRYR | 3 |
| SATR | 6 |
| SRYA | 5 |
| SRYR | 19 |

All individuals with A at position 127 have T at position 194.

3 SNP estimated frequencies

| | |
|---|---|
| CAA | 41% |
| CAG | 4% |
| CGA | 4% |
| CGG | 3% |
| GAA | 1% |
| GAG | 7% |
| GGA | 5 |
| GGG | 27% |

PBE137

| | |
|---|---|
| 1 | tgggttatng gctgttttga ttcyragcga aaatttgagg gaaacacaaa gatgaagtga |
| 61 | agctcttaat ttactttttc actttctata cttcctttaa aaatgacttc aataccctag |
| 121 | Yatttttcca tatgaacgtt tctatttttc atgattatta aagtggtaca aaattggaac |
| 181 | aactgcaaaa tccattggta agtgattgat tgtgatatag taatgcaatc tttatgaaga |
| 241 | ttaaaatgct ttattaccat aaggagatgt tcataatYta ttttgaagtg aaaaaagcag |
| 301 | gtggcacaat agaatatatt tgatgtgaaa ataaccatta acagtggtgg catgagtgaa |
| 361 | aggaaggttg tagagggact ttccaaggga cctaatcttc tatgcttgRa aaatctgtag |
| 421 | acaccttaag tgcaygtaac taacacctgg aggttgatct ttacactgrg gsnssaaara |
| 481 | aaaa |

SNPs to score 121, 278, 409, estimated frequencies:

| | |
|---|---|
| CCA | 22% |
| CCG | 17% |
| CTA | 5% |
| CTG | 5% |
| TCA | 5% |
| TCG | 4% |
| TTA | 5% |
| TTG | 36% |

Alternate SNP:

| position 435 | Estimated frequency |
|---|---|
| C | 77% |
| T | 23% |

PRKAG_STS3

| | |
|---|---|
| 1501 | ttaacaaaat cgatactatg tgtgtctgta cacttatgac tttggagtag aaacactggg |
| 1561 | ttggtttccc acaccttgga gtgcttgggg aggggtcacc tcagyacctc tggccaccag |
| 1621 | cagccttaga tctggaacaa atgtgcagac aaggatctcg tggagggcat gccaggacgt |
| 1681 | gggagaggca gacagcaggc tcatgtagag gcaggcccgg gaggcgcccg gtggaagaac |
| 1741 | cctggctggc aggggacctc tgaggcgcag ggaacgattc accctcaact gttctctccg |
| 1801 | gcgctcagat caagaaggcc ttctttgctt tggtggccaa cggcRtccga gcggcacctt |
| 1861 | tgtgggacag caagaagcag agcttcgtgg gtgaggaggg gctggggagg cagaggtggy |
| 1921 | ggggaaggga ataggggRac cttgtggggt gattctaggg ccgagctctg acayaccaca |
| 1981 | ggcttsaacc aagcaggggc ctggcctgg[rgrrscgga]gggagcatytga ccccggtctc |
| 2041 | cyggtggccR gctgggagat ctcaactgta ggagagctgt gaccagctga cccctccagc |
| 2101 | tctactaccc caaggtccct gtcgcaggtg ctaagtaaga agaggacagg cggaggaagg |
| 2161 | aagtcagaaa atagaagaag cagggcagga aggagagaaa tgacagggga agcataagag |
| 2221 | ggacaacccc atttstcagg cacgggaggg gctgccctcc tgtcctcttt tggccaccct |
| 2281 | cagtaaaagg atgtgggcag ggtgggggga ggggcccggg ctgaccccca ttgctccccc |
| 2341 | cyyttgcccc ccsyacaggg atgctgacca tcacagactt catcttggtg ctgcaccgct |
| 2401 | attacaggtc ccccctggtg aggagtggtc tgggggtcct ggaacaccca tctgggctgg |
| 2461 | ggtggaagga gttcagggga ccctcgcctg actttgggag ttccgttgct gtctttaggt |
| 2521 | ccagatctac gagattgaag aacataagat tgagacctgg aggggtgagc aggcgagggg |
| 2581 | acsgkcgaag gggctgaggg tgtgtgggtg aggatgrggc caaggacctc agggagagca |
| 2641 | tgcgcagtgg aggtttcctg gaggaagcgg gaggagggtg atcgggagcc caggggatct |
| 2701 | aagggaggga gacagtctgg gggtggccac gtgaggcggg gtggtcggcc cctttgtgct |
| 2761 | gattctggct tttcctgcag agatctacct tcaaggctgc ttcaagcctc tggtctccat |
| 2821 | ctctcccaat gacaggtgag cttccccagc crcccactcg agcctccttg ccccgcacag |
| 2881 | accccttctc cagctcatcg gttctaagct catggactca tcgtccgtgg actgcagatg |
| 2941 | cggcagcttt gacaccctgt cctcctctcc aggggggctg ggatgaaggg gctctctttc |

Three SNPs to score:

The sequence within [ ] is a complex in/del. The SNPs at position 1920, 1974, 1886 and the indel are carried on the same haplotype and can be used interchangeably.

| Position | 1845 | 1938 | 2050 | Estimated frequency |
|---|---|---|---|---|
| | G | G | A | 27% |
| | G | A | A | 10.5% |
| | G | A | G | 43% |
| | A | A | G | 19.5% |

Position 1845 splits the AG haplotype.

WSCR STS1

| | |
|---|---|
| 1 | acggacattc ctgacctcca ctctttggcc tgagggctgt gaccaaggga cagYRggcca |
| 61 | ccMggtggaa cYacaacagc cccagaYctc ccctgcgaag ggagtccagg tcctggggtc |
| 121 | ctaagggacccccc(C)tgccctgagcagcca atcaggccac gtgcacacgg ctaacctggg |
| 181 | gctcctccct tcccctctgcccgg(GATTC)gattctggaga Rcacctgcaa gcagcggtcc |
| 241 | cccccaggag acagacgggg gcggaagaag cctgcacagc aggacgtgtc tggggtgaat |
| 301 | YRgggactcc aagactg(GGAAGAAGGCTG)ttctcYgtgcc tagaacacat ctaggagcac |
| 361 | tgggggctca gaaaaggcaY gccctggScR tgRaactcctcaRcactt(ACT)gctcaaatg |
| 421 | ttacactctg gtacaaaggg aatcagaaRc tcaagaactc ccagggctga tacataRgca |
| 481 | atgatcttgY ggcaaactgt cYcctcctat gcggtttgac cttgtcctcc tgggcgcccc |
| 541 | ctgtcagccc tttggcRgtR gggttgggca tgagScctgc cacgggtggc caagaagcYg |
| 601 | tgggaaaggt gccctgcctt ggagtctggg tttgctcatc aatgaaatga gccaacatgg |
| 661 | accttgaggg tgtcgaggtg aggttgcYSa ggtgaggtgg ggcatcRtag gacctRcacc |
| 721 | gggtgtgggg cccaRagcca Ytcacgaggc ccttcccggg ccctgctaga gacacRccca |
| 781 | cactacctgg ggtctcggcc tctagatttg gagcaagacc aaccctggtg gcagYggcag |
| 841 | ccgctccagg gtcacgcccc tccagtcttc cagagcccgc tctggggacc cccacccccg |
| 901 | ggaaagtggc tgccaaacca ttgctcagtc tcctcaggac tggccgatgg gggctgcgct |
| 961 | cgctcaggtc ccctcggtgg gcgcgtgtgt tggggatggg cggggtttcc ctccacgtgt |
| 1021 | ttgtgagcca cacggactag cgccgggatg cctggcacag gtgccttggg gaggccc |

The sequences in ( ) are In/Dels.

SNPs

Two SNPs:

| 302 | 599 | Estimated frequency |
|---|---|---|
| A | C | 47% |
| G | C | 18% |
| G | T | 35% |

T at position 301 is low in frequency (˜3%)

Three SNPs:

| 135 | 392 | 599 | |
|---|---|---|---|
| T | G | C | 13% |
| C | A | C | 40% |
| C | G | T | 34% |

Frequency estimates do not include individuals heterozygous at position 135

Other Possible SNPs to score:

Position 222

| | |
|---|---|
| A | 36% |
| G | 64% |

Position 408 Indel

| | |
|---|---|
| A | low |
| G | high |

Position 560

| | |
|---|---|
| A | 87% |
| G | 13% |

Two SNPs:

| 55 | 63 | Estimated frequency |
|---|---|---|
| G | C | 44% |
| G | A | 34% |
| A | A | 22% |

Three SNPs:

| 55 | 63 | 135 (In/Del) | Estimated frequency |
|---|---|---|---|
| G | C | C | 44% |
| G | A | T | 18% |
| G | A | C | 16% |
| A | A | C | 22% |
| 55 | 63 | 302 | Estimated frequency |
| G | C | G | 44% |
| G | A | G | 18% |
| G | A | A | 16% |
| A | A | A | 22% |

BG_mod

| | |
|---|---|
| 1 | ccccagccct ttttccaggt cagcscaggg aaaaaacatg ttctctgtcc ctggttatac |
| 61 | tgtttagaaa catcacctcc ctcggcgwwa ctaaaacttg ggggttgcaa tttattcctt |
| 121 | gcttctttgt atttcktacc acattgagag agctctaggt tttcatccgc agattcccaa |
| 181 | accttcgcag aggagctgtt tcacaggacc gtgattcaag tttactctac ttttccatca |
| 241 | tttatttggt catatgttta aatgaagaaa gaaaggaatg aagatacctg aatgaaatga |
| 301 | gtatttgttt tcttaccagc aggactgaat acaaatgaag agaagaaaaa tacgcacatt |
| 361 | taggacttgg gcagaggttt tatccacgct ctccttgtgg ttatttccca tattcagaag |
| 421 | gcrcrggwgw ggattygtct gtatggtcct aaattgaacc acagtggtca aatccctcca |
| 481 | ctttctgctc cttggattct tcgtttgtgt actaagaaaa tggggaggca gtctctaaga |
| 541 | gattgctaca gtgggactca actctaaaag ttgtacagac ttgctaagga ggatgaaatt |
| 601 | agtagcactt tgcactgtga ggatggkacc tagagctccc cagagaaggg ctgaaggtct |
| 661 | gaagttggtg ccaggaacgt ctcgaagaca ggtatactgt caacattcaa gcctcaccct |
| 721 | gtggaaccac gccctggcct gggccaatct gctcccagaa gcagggaggg caggaggctg |
| 781 | ggggggcata aaaggaagag cagagccagc rgccacctac atttgcttct gacacaaccg |
| 841 | tgttcactag caactgmaca aacagacaac atggtgcatc tgtctgctga ggagaaggag |
| 901 | gccgtcctcg gcctgtgggg caaagtgaat gtggacgaag ttggtggtga ggccctgggc |
| 961 | aggttggtat ccagggcttc aggagaggga gygggaggtg ggcaggtggg gacagagcca |
| 1021 | cccctgcctt tctgacaggt gctgactccc tcgggcc[ttgcgctcttttcacccctcagg] |
| 1081 | ctgctggttg tctacccctg gactcagagg ttcttcgagt cctttgggga cctgtccaat |
| 1141 | gccgatgccg tcatgggcaa tcccaaggtg aaggcccacg gcaagaaggt gctccagtcc |
| 1201 | ttcagtgacg gcctgaaaca tctcgacaac ctcaagggca cctttgctaa gctgagYgag |
| 1261 | ctgcactgtg accagctgca cgtggatcct gagaacttca gggtgagtct gggggaccct |
| 1321 | caYgttctcc ttctgctcct tggtcatggc tgagctcgtg tcatgaagag agggcygaaa |
| 1381 | ggcaggatgc cgtttagaat gaaaaggaag cattctggtt acatMgctgg ggactctgca |
| 1441 | ggaccactga rtttcttttc cctcttctta ttcacagcca tcatttcctc ttactctctc |
| 1501 | ttgttctcck ctgttttgtt tkgtttkgtt tkgktttgtt ttgttttttt ttttttcctc |
| 1561 | tgcgatgtct tctctctttt aagttaaact ttttgagtgt tttgttaaaa aaaaaaaaac |
| 1621 | tttcttcttt taagccactt taaaaaatgt tatctaacag ttgcccctta ttctttcctt |
| 1681 | ttgaggcaag aaggataaaa tgtttattgc ttcctggcat ggttctaaag cataaaaagg |
| 1741 | ataacataaa ttcaagacta aggtagaaag agagaaacat tyctagctgt cattcaggtt |
| 1801 | gccatgggtg gattcacatc aata[gtaacgtctacacggcagctcact]tt ctgtttatct |
| 1861 | tctaggggct cagcttggga tgagactgaa atactcctgg gtctaagctg gtcctctact |
| 1921 | aatcaggccc ttccttttta tctctctccc acagctcctg ggcaaYgtga tagtggttgt |
| 1981 | tctggctcgc cgccttggcc atgacttcaa cccggatgtg caggctgctt ttcagaaggt |
| 2041 | ggtggctggt gtggccaatg ccctggccca caagtaccac taagttcccc ttcctgattt |
| 2101 | ccaggaggag ccctttttcc tctgaaccca aaaactggat atggaaatat tatgaagagt |
| 2161 | tttgagcatc tggcctctgy gtaataaaaa catttatttt cattgcactg gttttttaaa |
| 2221 | attatttcag tgtctctcac tcagatgggc atatgggaat gaaaggtatt ataaaaaaat |
| 2281 | rtaaataaat gaggggctaa ttgagacttt aagaaagctc tctgtacctt ggaccccatg |
| 2341 | aaagragtgg ttgtaaagca cgcatgtgag tgggaataca ccttatacct cttcctttcc |
| 2401 | t |

The reduced complexity sequence between [ ]should not be used for the primer design.

Two SNPs:

| Position 1323 | 1425 | Estimated frequency |
|---|---|---|
| C | A | 30% |
| C | C | 46% |
| T | C | 24% |

Three SNPs:

| Position 1257 | 1323 | 1425 | Estimated frequency |
|---|---|---|---|
| C | C | A | 30% |
| C | C | C | 35% |
| C | T | C | 24% |
| T | C | C | 11% |

An alternative is to score the following positions:

| 1323 | 1425 | 1966 | Estimated frequency |
|---|---|---|---|
| C | C | T | 11% |
| C | C | C | 35% |
| C | A | C | 30% |
| T | C | C | 24% |

| | |
|---|---|
| 1 | ggaatttcga tggctctagt acttttcagt ctggaggctc |
| | caacagtgac atgtatcttg |
| 61 | accctgctgc catgtttcgg gaccctttcc gcaaggaccc |
| | caacaagctg gtgttctgtg |
| 121 | aggtcttcaa gtacaaccga aagcctgcag gtgggtagag |
| | gataggtctg ggtgccccca |
| 181 | ccccaatcca tgaattggaa aggcggggaa gaggctattc |
| | caaaattggt actgagaaga |
| 241 | ccagcctgtc ttagaactgt atgtgaagtc tgtattcgct |
| | tgggcctcat ctctccatct |
| 301 | agaacatcct tgttcaaaga ggttgcgtgg cccatcctcg |
| | cattattggt ggcataaaac |
| 361 | aacattcaat ttctctctct ctctctctct ctcttctttt |
| | ttttttttgc tttttagggc |
| 421 | tgcacccgtg gcatatggag gttctcaggc taggggtcca |
| | atcagagcta cagctgccag |
| 481 | ccacagccat agcaacgcag gatccaagcc ccgtctgtga |
| | cctacatctg tgacctacac |
| 541 | catagctcac agcagtgccg gatccttaac ccattgagca |
| | aggccaggta ttgaacccac |
| 601 | aacctcatgg ttcttagtcg gattcatttc cgctacacca |
| | cggcgggaac tccaacatta |
| 661 | gattcttcta aatctgcttg ccgttagtcc tctgtggcac |
| | agccctcctg cctgcctgta |
| 721 | gattttaaga acacagattc taaagttaga atgttggatt |
| | tgaattctag ttgtgccact |
| 781 | tccttgctRt gaacttgggc aagttacttt tcacctctac |
| | tcctcacttc cctgatttta |
| 841 | aaatggggaa attaataaaa tccatcttag aaagttgttt |
| | Rtggggcgaa ctgagttgat |
| 901 | gagtgcggat aattgagaag gctcgtacac cacgcactRg |
| | ttgccttttc attccacaca |
| 961 | gcgcgtttcc tttctctgcc cacggactaa aatgaggaca |
| | tccggcttca ccaataacat |
| 1021 | acgtttgcct tgagggttgc tggagccccg cccctttgSt |
| | gtttgtctag cacacccatt |
| 1081 | gctcccagtt ctacttcgtt cgtcttttta aatttgccat |
| | gctctgccag tgagactttt |
| 1141 | tggcaagtca cttagtgact ctggacctta ttacttgtct |
| | ttgtttggag aggcattatc |
| 1201 | atattgtggt aaataacttg agctcggaag ttgagacaag |
| | cagagtttcc tggctctgct |
| 1261 | gcttttgctg tgaccYgggc aggctacctg ccatctctga |
| | gcctcatttt ctcatttgta |
| 1321 | aaaacggaga ctcgtaatcc ccacctcagg gttgttagga |
| | agactcaaga ttataggttt |
| 1381 | tctttgataa aatatgaagg tcataaaatg gttcctggga |
| | catagtggaa tagttatagt |
| 1441 | aatgagtcca taaagtaatt ctagcttaga aatagtttca |
| | gttgagttca ttgcatttta |
| 1501 | ctggggtttg gagggggttt gtttgctttt gcaccagcca |
| | atactataga actttgtagt |
| 1561 | ggtttacctt ctcaccttga atgtaaggct cttcttctct |
| | cagtcaaaat tgtagatttR |
| 1621 | gctctaaatg cctgagaaac taggaataga tggaaagcga |
| | gaRgcagttc tctagtgaat |
| 1681 | agagagttta agtgcttgac atttggcgtt tgggaggggg |
| | ccagcttgca gataaagtga |
| 1741 | aaacgtgctc tattctggat ccactgccgt gtgactttgt |
| | tgtaacccag tttagttata |
| 1801 | aataaagcct tttgagagtc tccctacaga tacataatta |
| | tagcttctct cattgtccRc |
| 1861 | ttttacacca gcggggactg aaagcttaca gtgcagcYRa |
| | cactcatcat ctcaggtgtg |
| 1921 | Yggattcctg gaaaccacct caggcgcatg tgcattcggt |
| | acatttgccc atgcgtctct |
| 1981 | aataagtgtg ttggctcttt ctttttccta gagaccaact |
| | taaggcacac ctgtaaacgg |
| 2041 | ataatggaca tgg |

1 SNP

| 789 | |
|---|---|
| G: | 90% |
| A: | 10% |
| 939 | |
| G: | 18.3% |
| A: | 81.7% |
| 1069 | |
| G: | 81.6% |
| C: | 18.4% |
| 1276 | |
| C: | 80% |
| T: | 20% |
| 1620 | |
| G: | 93.1% |
| A: | 6.9% |
| 1663 | |
| G: | 7.8% |
| A: | 92.2% |
| 1859 | |
| G: | 88.9% |
| A: | 11.1% |

IL2R

| | |
|---|---|
| 1861 | ctcctgctcc gacttttaaa tgaattgcct tccggacaca aagaagtggc gggctgccga |
| 1921 | gctgacgggg ctcatcattc atggtcgcac tccttgccat gtgtgatgcc rtcaggcggm |
| 1981 | cggccrgaat ggagtccctc tgggggagac tmatacacaa attccatcag aaaatctcag |
| 2041 | ttgagaaata catcRgaggc catggtcRtc tggacaggaa gcccgactga caggctggcr |
| 2101 | ctgggctgac catgaacagr cacaaccaag agccccccca agtgcttatc tttatgaccc |
| 2161 | cagtacccca atattggctg gcaggtcgtg ggctctaaaa acaccttctt tttgaatgaa |
| 2221 | tacacgaagg gcattttctc ttgtacacat ttgtgaagtt attttcttca tttcacccct |
| 2281 | actcccyagc ctccccgagg atccccgtaa aacaagttac tcctagacct gaagaacaga |
| 2341 | aggaacgaaa aaccacagaa actcagggcc agatgcagcc cccgaaccaa gctaatcttc |
| 2401 | caggtaagag gcaaaccatc cccctgcagg tagaggagat gctgccctca ggaggcgtct |
| 2461 | gtcctctttc ctaaggatgc crtgtggaca agaagcgggt ttgcargctg gtcactcagc |
| 2521 | acatcaagcc actctgacag cagccaacct gggttcaggg cttcctctgg gtttgggaac |
| 2581 | cagccaaacr gaattcaact ccattcaatc caactcaact cactkcactt caccaagcac |
| 2641 | atctggggac tacctgcggc caggagatgc aggcctgacc agacagtggt ccccgtcctc |

MCP

2 SNPs:

| | | | |
|---|---|---|---|
| Positions | 2055 | 2069 | Estimated Heterozygosity |

| | | |
|---|---|---|
| A | G | 37% |
| G | A | 39.1% |
| G | G | 23.9% |

| | |
|---|---|
| 1 | ctccctcmac ctacmawhwt tcctaaggtt tgcagaggag ctgccataga gctcaaaaca |
| 61 | yggtctacag acaagcatct tctccatccc tyctcatctt ckcacRggcc gYttgacaac |
| 121 | atctctwgng gakgkggdgg rgcngncscy vcncvinsckg kdwrwcsmcc yygcgtyyac |
| 181 | scmaagmgct ctgactctgg agttctagtc ctcgcgggac cttaggaagt tcacggtcaa |
| 241 | tactcygccc ttgggctcag wcactaagak gatctccggg taaagagata gacagtagct |
| 301 | ccatgcctga tttaggaaaa ctgtccgtac agacagttgt aattcatttc ctttcagaga |
| 361 | caaatcctgc tctcttccta gttcctgaag tcattaaaat caaaagctct cagaaacgtc |
| 421 | ccagcatttg ctaagtccac gctgggggag gatgggcaga gccgtgttca gcarcgtttg |
| 481 | acagcaacac ccacttattt tcattcagta tccataggca tatatcatgc acctggtata |
| 541 | ggyctctctc tcagcactgg agatacagca aagaaaacgc tattcctgcy ccatggagct |
| 601 | tgnttcagaa aaataractc aaaacctttc aaatggagct gccgctgggc caagtcaaaa |
| 661 | taagtaaaag aaatccgtga gaaacccttc agttatatta agaagaaata gcttgatg |

One SNP:

| Position 102, | Estimated frequency |
|---|---|
| G | 16% |
| A | 84% |

One SNP:

| Position 106, | Estimated frequency |
|---|---|
| G | 47% |
| A | 53% |

2 SNPs:

| 102 | 106 | Estimated frequency |
|---|---|---|
| G | G | 15% |
| T | A | 63% |
| T | G | 22% |

mtDNA (15001-16585)

| | |
|---|---|
| 15001 | ggatacatct cagtagccat agcagtagta taaccaaaaa ccaccaacat accccccaaa |
| 15061 | taaatcaaaa acgccattaa acctaaaaaa gacccaccaa aattcaatac aataccacaa |
| 15121 | ccaactccac cacttacaat caacccaagt ccaccataaa taggagaggg cttagaagaa |
| 15181 | aaaccaacaa acccaataac aaaaatagta cttaaaataa atgcaatata cattgtcatt |
| 15241 | attctcacat ggaatttaac cacgaccaat gacatgaaaa atcatcgttg tacttcaact |
| 15301 | acaagaacct taatgaccaa catccgaaaa tcacacccac taataaaaat tatcaacaac |
| 15361 | gcattcattg acctcccagc cccctcaaac atctcatcat gatgaaactt cggttccctc |
| 15421 | ttaggcatct gcctaatctt gcaaatccta acaggcctgt tcttagcaat acattacaca |
| 15481 | tcagacacaa caacagcttt ctcatcagtt acacacattt gtcgagacgt aaattacgga |
| 15541 | tgagttattc gctatctaca tgcaaacgga gcatccatat tctttatttg cctattcatc |
| 15601 | cacgtaggcc gaggtctata ctacggatcc tatatattcc tagaaacatg aaacattgga |
| 15661 | gtagtcctac tatttaccgt tatagcaaca gccttcatag gctacgtcct gccctgagga |
| 15721 | caaatatcat tctgaggagc tacggtcatc acaaatctac tatcagctat cccttatatc |
| 15781 | ggaacagacc tcgtagaatg aatctgaggg ggcttttccg tcgacaaagc aaccctcaca |
| 15841 | cgattcttcg ccttccactt tatcctgcca ttcatcatta ccgccctcgc agccgtacat |
| 15901 | ctcctattcc tgcacgaaac cggatccaac aaccctaccg gaatctcatc agacatagac |
| 15961 | aaaattccat ttcacccata ctacactatt aaagacattc taggagcctt atttataata |
| 16021 | ctaatcctac taatccttgt actattctca ccagacctac taggagaccc agacaactac |
| 16081 | accccagcaa acccactaaa caccccaccc catattaaac cagaatgata tttcttattc |
| 16141 | gcctacgcta ttctacgttc aattcctaat aaactaggtg gagtgttggc cctagtagcc |
| 16201 | tccatcctaa tcctaatttt aatgcccata ctacacacat ccaaacaacg aggcataata |
| 16261 | tttcgaccac taagtcaatg cctattctga atactagtag cagacotoat tacactaaca |
| 16321 | tgaattggag gacaacccgt agaacacccg ttcatcatca tcggccaact agcctccatc |
| 16381 | ttatacttcc taatcattct agtattgata ccaatcacta gcatcatcga aaacaaccta |
| 16441 | ttaaaatgaa gagtcttcgt agtatataaa ataccctggt cttgtaaacc agaaaaggag |
| 16501 | ggccacccct ccccaagact caaggaagga gactaactcc gccatcagca cccaaagctg |
| 16561 | aaattctaac taaattattc cctgc |

The swine mtDNA sequence is publicly available. For example complete mtDNA sequence of Sus scrofa breed Duroc can be obtained from GenBank with accession number AY337045; and breed Landerace (AF486866).

I. SNP Detection Methodology

Detection of SNPs in a high throughput mode or in a smaller scale can be performed using standard SNP detection techniques that include specific PCR amplification followed by sequencing, mass spectrometric analysis, and HPLC based analysis. Some high throughput SNP detection techniques and devices are described in U.S. Pat. Nos. 6,720,143, 6,537,748, 6,337,188, and 6,225,109, which are herein incorporated by reference.

### DOCUMENTS

Garber R. A. & Morris J. W. (1983) In: Inclusion Probabilities in Parentage Testing, pp. 277-80.
Hawken et al., (2004), An interactive bovine in silico database (IBISS). Mammalian Genome 15, 819-827
Jamieson A. (1965) Heredity 20, 419-41.
Jamieson A. (1994) Animal Genetics 25, Supplement 1, 37-44.
Weir B. S. (1996) Genetic Data Analysis II. Sinauer Associates Inc., Sunderland, Mass., USA.

1. A method for identification of an animal, the method comprising:
(a) obtaining a sample from the animal or from a processed product of the animal;
(b) performing single nucleotide polymorphism (SNP) analysis comprising analysis of a plurality of markers, wherein a marker comprises at least one SNP;
(c) generating SNPTracks of the animal from (b); and
(d) comparing the SNPTracks of the animal to a database comprising a plurality of SNPTracks to identify the animal.

2. The method of claim 1, wherein the animal is a farm animal selected from the group consisting of cow, pig, sheep, and poultry.

3. The method of claim 1, wherein the animal is a mammal.

4. The method of claim 3, wherein the mammal is a human.

5. The method of claim 1, wherein the plurality of markers are selected from the group consisting of autosomes, sex chromosomes, and mitochondrial DNA.

6. The method of claim 1, wherein a plurality of SNPs in a marker are present within a nucleotide region of 0.2 to about 10 kb.

7. The method of claim 6, wherein the plurality of SNPs in a marker are present within a nucleotide region of 0.2 to about 1 kb.

8. The method of claim 1, wherein the plurality of markers are selected from the group consisting of swine markers designated ACY-STS7; COX2; EG-STS7; GALT; IKBA; LEPR; P450-STS18; PBE3; PBE42; PBE43; PBE 57; PBE59; PBE 64; PBE 73; PBE 84; PBE132; PBE137; PRKAG-STS3; RYRA-STS6; SCAMP; VAN-STS1; WSCR-STS1; BG; AMG; CTSL; PBE112; and MYF5.

9. The method of claim 8, wherein the plurality of markers comprise SNPs at ACY-STS7 (245 G/C 421 C/T); COX2 (368 C/T 533 G/A 939 C/T); EG-STS7 (774 G/A 805 G/A 817 G/A); GALT (478 G/A 758 C/G 866 C/G); IKBA (4476 C/T 4679 T/A 4904 G/T); LEPR (426 A/C/G 810 G/C); P450-STS18 (71 C/T 138 G/A 361 G/A); PBE3 (115 G/A 192 A/T 555 T/G); PBE42 (111 G/A 118 T/G 181 C/T); PBE43 (314 C/T 471 C/A 524 C/T); PBE 57 (75 C/T 109 G/A 197 C/T 268 T/G); PBE59 (276 C/T 494 C/T); PBE 64 (115 G/A 419 C/G 515 C/T); PBE 73 (93 A/C 116 A/G 177 C/T 477 C/T); PBE 84 (14 A/G 97 T/C 428 T/C); PBE132 (102 C/G 127 A/G 193 C/T 371 A/G); PBE137 (121 C/T 278 C/T 409 A/G); PRKAG-STS3 (1845 G/A 1938 G/A 2050 G/A); RYRA-STS6 (402 A/C 408 C/G 567 A/C); SCAMP (184 C/T 389 G/A 516 C/T 582 G/A 939 A/T); VAN-STS1 (889 C/T 950 C/T 1009 G/A 1065 C/T); WSCR-STS1 (411 C/T 599 C/T); BG (1257 C/T 1323 C/T 1425 C/A 1966 C/T); AMG (907 A/C 975 A/G 1467 A/G); LCN (87 C/T 373 G/C 402 C/T); CTSL (252 A/G 272 A/G); PBE112 (61 C/T 87 C/T); and MYF5 (1833 A/G 2204 C/G 2335 A/C).

10. The method of claim 1, wherein the plurality of markers are selected from the group consisting of swine mitochondrial markers designated at positions 15543 (C/T), 15558 (A/T), 15615 (C/T), 15616 (C/T), 15675 (C/T), 15714 (C/T), 15840 (C/T), and 16127 (G/A).

11. A system for identification of an animal from which a sample is derived, the system comprising:
(a) obtaining a sample from the animal or from a processed product of the animal;
(b) performing single nucleotide polymorphism tracking (SNPTrack) analysis of the sample comprising a plurality of markers, wherein the plurality of markers comprise at least one SNP;
(c) storing one or more SNPTracks of the sample in a computer system;
(d) comparing the one or more SNPTracks of the sample with known SNPTracks in a database; and
(e) identifying the animal.

12. A computer system for identification of a sample, the computer system comprising:
(a) a software module comprising instructions operative to provide a searchable database comprising a plurality of SNPTracks of a plurality of animals, the SNPTracks comprising a plurality of markers, wherein the plurality of markers comprise at least one SNP;
(b) a software module comprising instructions operative to provide an algorithm to determine exclusion probabilities; and
(c) a software module comprising instructions operative to provide an interface to accept and compare a query SNPTrack with the plurality of SNPTracks in the database to identify the sample.

13. A method of developing a database for identification of an animal or a product derived from the animal, the method comprising:
(a) performing SNPTrack analysis of a plurality of samples obtained from a plurality of animals from one or more sources with a plurality of markers, wherein the plurality of markers comprise one SNP;
(b) obtaining and storing the SNPTracks of the plurality of samples in a database, wherein the database is searchable;
(c) performing SNPTrack analysis of the product;
(d) comparing one or more SNPTracks of the product with the SNPTracks of the plurality of the samples stored in the database; and
(e) identifying the product.

14. The method of claim 13, wherein the database further comprises data selected from the group consisting of production farm, farm of origin, retail outlet, wholesaler, breeding record, animal identification, offspring data, sibling data, and lineage data.

15. The method of claim 13, wherein the one or more sources are selected from the group consisting of farm of origin, production farm, processing center, retail outlet, distribution center, and wholesaler.

16. The method of claim 13, wherein the SNPTracks of the plurality of the samples stored in the database are associated with an animal identification system.

17. The method of claim 13, wherein the SNPTrack analysis is performed on the plurality of samples between birth and slaughter of the plurality of the animals.

18. The method of claim 13, wherein the database has limited access to an authorized user.

19. A method of obtaining a SNPTrack of a sample, the method comprising:
(a) selecting a first marker such that the first marker has at least one SNP, wherein more than one SNP are within a 0.2 to about 10 kb region in the genome;
(b) selecting a second marker such that the second marker has at least one SNP, wherein more than one SNP are within a 0.2 to about 10 kb region in the genome;
(c) performing SNPTrack analysis of the sample with the first and second markers; and
(d) obtaining the SNPTrack of the sample.

20. The method of claim 18 further comprising performing a SNPTrack analysis with a plurality of markers and obtaining the SNPTrack for the plurality of markers.

21. A nucleotide segment comprising a plurality of SNPs, wherein the segment is amplified with primers listed in TABLE 3.

22. The nucleotide segment of claim 21, wherein the nucleotide segment is selected from the group consisting of autosomes, sex chromosomes, and mitochondrial DNA.

23. A high-throughput system for tracking an animal and a meat product through a supply chain, the system comprising:
(a) performing SNPTrack analysis of a plurality of samples obtained from a plurality of animals;
(b) obtaining and storing a plurality of SNPTracks in a database, wherein the database is searchable;
(c) obtaining a plurality of samples from meat products;
(d) performing SNPTrack analysis of the plurality of samples from meat products; and
(e) tracing the plurality of samples from meat products to a location in the supply chain.

24. The high-throughput system of claim 23, wherein the plurality of samples from the plurality of animals are obtained prior to slaughtering in a farm or a processing center.

25. The high-throughput system of claim 23, wherein the supply chain comprises identification of animals and their products from birth to post-slaughter.

26. The high-throughput system of claim 23, wherein the supply chain comprises from a farm of origin to a consumer.

27. A software module comprising instructions operative to provide a database comprising SNPtracks identified in TABLE 7.

28. A SNPTrack analysis kit to identify an animal comprising:
(a) a plurality of oligonucleotides corresponding to a plurality of markers that contain at least one SNP, wherein more than one SNP are within a 0.2 to about 10 kb region of each marker;
(b) reagents to perform SNPTrack analysis; and
(c) access to compare SNPTracks with a plurality of SNPTracks in a database to identify the animal.

29. The kit of claim 25 further comprising an instruction manual to identify the animal.

30. A method of tracing an infected meat sample to a particular location, the method comprising:
(a) performing SNPTrack analysis of a plurality of samples obtained from a plurality of animals with a plurality of markers, wherein the plurality of markers comprise at least one SNP;
(b) obtaining and storing the SNPTracks of the plurality of samples in a database, wherein the database is searchable;
(c) performing SNPTrack analysis of the infected meat sample;
(d) comparing one or more SNPTracks of the infected meat sample with the SNPTracks of the plurality of the samples stored in the database; and
(e) tracing the infected meat sample to a particular location.

31. The method of claim 30 wherein the infected meat sample is a beef sample infected with Mad Cow Disease or bovine spongiform encephalopathy (BSE).

32. The method of claim 30 wherein the infected meat sample is a sheep or goat sample infected with scrapie disease.

33. The method of claim 30 wherein the infected meat sample is an infected pork sample.

34. The method of claim 30 wherein the infected meat sample is obtained from a meat product in the market.

35. The method of claim 30 wherein the particular location is selected from the group consisting of farm of origin, production farm, processing center, retail outlet, distribution center, and wholesaler.

36. A method of assuring food safety of a meat product, the method comprising:
(a) performing SNPTrack analysis of a plurality of samples obtained from a plurality of animals prior to slaughtering with a plurality of markers, wherein the plurality of markers comprise at least one SNP;
(b) obtaining and storing a plurality of SNPTracks of the plurality of samples in a database, wherein the database is searchable;
(c) tracing the meat product to its source by performing SNPTrack analysis of the meat product and comparing a SNPTrack of the meat product with the SNPTracks of the plurality of samples stored in the database; and
(d) determining if the meat product from the source is safe.
